SEQUENCE LISTING (29 sequences)

SEQ ID NO: 1 - DNA, 4498 bp, Artificial
  Homo sapiens transmembrane protein
agaggccgccggggccgtggatggggagggcgcgccgcccggcggtcccagcgcacaggcggccacgatgagggtcaacgagaagtactcgacgctcccggccgaggaccgcagcgtccacatcatcaacatctgcgccatcgaggacatcggctacctgccgtccgagggcacgctgctgaactccttatctgtggaccctgatgccgagtgcaagtatggcctgtacttcagggacggccggcgcaaggtggactacatcctggtgtaccatcacaagaggccctcgggcaaccggaccctggtcaggagggtgcagcacagcgacaccccctctggggctcgcagcgtcaagcaggaccaccccctgccgggcaagggggcgtcgctggatgcaggctcgggggagcccccgatggactaccacgaggatgacaagcgcttccgcagggaggagtacgagggcaacctcctggaggcgggcctggagctggagcgggacgaggacactaaaatccacggagtcgggtttgtgaaaatccatgccccctggaacgtgctgtgcagagaggccgagtttctgaaactgaagatgccgacgaagaagatgtaccacattaatgagacccgtggcctcctgaaaaaaatcaactctgtgctccagaaaatcacagatcccatccagcccaaagtggctgagcacaggccccagaccatgaagagactctcctatcccttctcccgggagaagcagcatctatttgacttgtctgataaggattcctttttcgacagcaaaacccggagcacgattgtctatgagatcttgaagagaacgacgtgtacaaaggccaagtacagcatgggcatcacgagcctgctggccaatggtgtgtacgcggctgcatacccactgcacgatggagactacaacggtgaaaacgtcgagttcaacgacagaaaactcctgtacgaagagtgggcacgctatggagttttctataagtaccagcccatcgacctggtcaggaagtattttggggagaagatcggcctgtacttcgcctggctgggcgtgtacacccagatgctcatccctgcctccatcgtgggaatcattgtcttcctgtacggatgcgccaccatggatgaaaacatccccagcatggagatgtgtgaccagagacacaatatcaccatgtgcccgctttgcgacaagacctgcagctactggaagatgagctcagcctgcgccacggcccgcgccagccacctcttcgacaaccccgccacggtcttcttctctgtcttcatggccctctgggctgccaccttcatggagcactggaagcggaaacagatgcgactcaactaccgctgggacctcacgggctttgaagaggaagaggaggctgtcaaggatcatcctagagctgaatacgaagccagagtcttggagaagtctctgaagaaagagtccagaaacaaagagactgacaaagtgaagctgacatggagagatcggttcccagcctacctcactaacttggtctccatcatcttcatgattgcagtgacgtttgccatcgtcctcggcgtcatcatctacagaatctccatggccgccgccttggccatgaactcctccccctccgtgcggtccaacatccgggtcacagtcacagccaccgcagtcatcatcaacctagtggtcatcatcctcctggacgaggtgtatggctgcatagcccgatggctcaccaagatcgaggtcccaaagacggagaaaagctttgaggagaggctgatcttcaaggctttcctgctgaagtttgtgaattcctacacccccatcttttacgtggcgttcttcaaaggccggtttgttggacgcccgggcgactacgtgtacattttccgttccttccgaatggaagagtgtgcgccagggggctgcctgatggagctatgcatccagctcagcatcatcatgctggggaaacagctgatccagaacaacctgttcgagatcggcatcccgaagatgaagaagctcatccgctacctgaagctgaagcagcagagcccccctgaccacgaggagtgtgtgaagaggaaacagcggtacgaggtggattacaacctggagcccttcgcgggcctcaccccagagtacatggaaatgatcatccagtttggcttcgtcaccctgtttgtcgcctccttccccctggccccactgtttgcgctgctgaacaacatcatcgagatccgcctggacgccaaaaagtttgtcactgagctccgaaggccggtagctgtcagagccaaagacatcggaatctggtacaatatcctcagaggcattgggaagcttgctgtcatcatcaatgccttcgtgatctccttcacgtctgacttcatcccgcgcctggtgtacctctacatgtacagtaagaacgggaccatgcacggcttcgtcaaccacaccctctcctccttcaacgtcagtgacttccagaacggcacggcccccaatgaccccctggacctgggctacgaggtgcagatctgcaggtataaagactaccgagagccgccgtggtcggaaaacaagtacgacatctccaaggacttctgggccgtcctggcagcccggctggcgtttgtcatcgtcttccagaacctggtcatgttcatgagcgactttgtggactgggtcatcccggacatccccaaggacatcagccagcagatccacaaggagaaggtgctcatggtggagctgttcatgcgggaggagcaagacaagcagcagctgctggaaacctggatggagaaggagcggcagaaggacgagccgccgtgcaaccaccacaacaccaaagcctgcccagacagcctcggcagcccagcccccagccatgcctaccacgggggcgtcctgtagctatgccagcggggctgggcaggccagccgggcatcctgaccgatgggcaccctctcccagggcaggcggcttcccgctcccaccagggcccggtgggtcctgggttttctgcaaacatggaggaccactttctgataggacattttcctttcttctttctgttttctttcccttgtttttgcacaaagccattatgcagggaatattttttaatctgtagtattcaagatgaatcaaaatgatggctggtaatacggcaataaggtagcaaaggcaggtgctttgcagaaagaatgcttggaaacttgagtctccctagaggtgaaaagtgagcagaggcccctagaaaccctcctctgaatcctcctaattccttaagatagatgcaaaatggtaagccgaggcatcgcgcaaaagctggtgcgatgcttcagggaaaatggaaaacccacgcaagaataatgattgattccggttccaaaaggtgtcacctacctgtttcagaaaagttagactttccatcgccttttccttccatcagttgagtggctgagagagaagtgcctcatccctgagccacacagggggcgtgggagcatcccagttatccctggaaagctagaaggggacagaggtgtccctgattaagcaggaaacagcacccttggcgtccccagcaggctccccactgtcagccacacacctgcccccatcacaccaagccgacctcagagttgttcatcttccttatgggacaaaaccggttgaccagaaaatgggcagagagagatgacctcggaagcatttccacagatggtgtcagggtttcaagaagtcttagggcttccaggggtcccctggaagctttagaatatttatgggtttttttttcaaatatcaattatatggtagattgaggattttttttctgtagctcaaaggtggagggagtttattagttaaccaaatatcgttgagaggaatttaaaatactgttactaccaaagatttttattaataaaggcttatattttggtaacacttctctatatttttactcacaggaatgtcactgttggacaattattttaaaagtgtataaaaccaagtctcataaatgatatgagtgatctaaatttgcagcaatgatactaaacaactctctgaaatttctcaagcaccaagagaaacatcattttagcaaaggccaggaggaaaaatagaaataaatttgtcttgaagatctcattgatgtgatgttacattccctttaatctgccaactgtggtcaaagttcataggtgtcgtacatttccattatttgctaaaatcatgcaatctgatgcttctcttttctcttgtacagtaagtagtttgaagtgggttttgtatataaatactgtattaaaaattaggcaattaccaaaaatccttttatggaaaccatttttttaaaaagtgaatgtacacaaatccacagaggactgtggctggacattcatctaaataaatttgaatatacgaaaaaaaaaaaaaaaaaaaaaaaa

SEQ ID NO: 2 - DNA, 3052 bp, Artificial
  Homo sapiens cDNA
ggatgaaaacatccccagcatggagatgtgtgaccagagacacaatatcaccatgtgcccgctttgcgacaagacctgcagctactggaagatgagctcagcctgcgccacggcccgcgccagccacctcttcgacaaccccgccacggtcttcttctctgtcttcatggccctctgggctgccaccttcatggagcactggaagcggaaacagatgcgactcaactaccgctgggacctcacgggctttgaagaggaagaggaggctgtcaaggatcatcctagagctgaatacgaagccagagtcttggagaagtctctgaagaaagagtccagaaacaaagagactgacaaagtgaagctgacatggagagatcggttcccagcctacctcactaacttggtctccatcatcttcatgattgcagtgacgtttgccatcgtcctcggcgtcatcatctacaggatctccatggccgccgccttggccatgaactcctccccctccgtgcggtccaacatccgggtcacagtcacagccaccgcggtcatcatcaacctagtggtcatcatcctcctggacgaggtgtatggctgcatagcccgatggctcaccaagatcgaggtcccaaagacggagaaaagctttgaggagaggctgatcttcaaggctttcctgctgaagtttgtgaattcctacacccccatcttttacgtggcgttcttcaaaggccggtttgttggacgcccgggcgactacgtgtacattttccgttccttccgaatggaagagtgtgcgccagggggctgcctgatggagctatgcatccagctcagcatcatcatgctggggaaacagctgatccagaacaacctgttcgagatcggcatcccgaagatgaagaagctcatccgctacctgaagctgaagcagcagagcccccctgaccacgaggagtgtgtgaagaggaaacagcggtacgaggtggattacaacctggagcccttcgcgggcctcaccccagagtacatggaaatgatcatccagtttggcttcgtcaccctgtttgtcgcctccttccccctggccccactgtttgcgctgctgaacaacatcatcgagatccgcctggacgccaaaaagtttgtcactgagctccgaaggccggtagctgtcagagccaaagacatcggaatctggtacaatatcctcagaggcattgggaagcttgctgtcatcatcaatgccttcgtgatctccttcacgtctgacttcatcccgcgcctggtgtacctctacatgtacagtaagaacgggaccatgcacggcttcgtcaaccacaccctctcctccttcaacgtcagtgacttccagaacggcacggcccccaatgaccccctggacctgggctacgaggtgcagatctgcaggtataaagactaccgagagccgccgtggtcggaaaacaagtacgacatctccaaggacttctgggccgtcctggcagcccggctggcgtttgtcatcgtcttccagaacctggtcatgttcatgagcgactttgtggactgggtcatcccggacatccccaaggacatcagccagcagatccacaaggagaaggtgctcatggtggagctgttcatgcgggaggagcaagacaagcagcagctgctggaaacctggatggagaaggagcggcagaaggacgagccgccgtgcaaccaccacaacaccaaagcctgcccagacagcctcggcagcccagcccccagccatgcctaccacgggggcgtcctgtagctatgccagcggggctgggcaggccagccgggcatcctgaccgatgggcaccctctcccagggcaggcggcttcccgctcccaccagggcccggtgggtcctgggttttctgcaaacatggaggaccactttctgataggacattttcctttcttctttctgttttctttcccttgtttttgcacaaagccattatgcagggaatattttttaatctgtagtattcaagatgaatcaaaatgatggctggtaatacggcaataaggtagcaaaggcaggtgctttgcagaaagaatgcttggaaacttgagtctccctagaggtgaaaagtgagcagaggcccctagaaaccctcctctgaatcctcctaattccttaagatagatgcaaaatggtaagccgaggcatcgcgcaaaagctggtgcgatgcttcagggaaaatggaaaacccacgcaagaataatgattgattccggttccaaaaggtgtcacctacctgtttcagaaaagttagactttccatcgccttttccttccatcagttgagtggctgagagagaagtgcctcatccctgagccacacagggggcgtgggagcatcccagttatccctggaaagctagaaggggacagaggtgtccctgattaagcaggaaacagcacccttggcgtccccagcaggctccccactgtcagccacacacctgcccccatcacaccaagccgacctcagagttgttcatcttccttatgggacaaaaccggttgaccagaaaatgggcagagagagatgacctcggaagcatttccacagatggtgtcagggtttcaagaagtcttagggcttccaggggtcccctggaagctttagaatatttatgggtttttttttcaaatatcaattatatggtagattgaggattttttttctgtagctcaaaggtggagggagtttattagttaaccaaatatcgttgagaggaatttaaaatactgttactaccaaagatttttattaataaaggcttatattttggtaacacttctctatatttttactcacaggaatgtcactgttggacaattattttaaaagtgtataaaaccaagtctcataaatgatatgagtgatctaaatttgcagcaatgatactaaacaactctctgaaatttctcaagcaccaagagaaacatcattttagcaaaggccaggaggaaaaatagaaataaatttgtcttgaagatctc

SEQ ID NO: 3 - DNA, 2118 bp, Artificial
  Homo sapiens cDNA
acgcacactgagtccaggtcctcctgaaggtcaccagcacagcggggccctgtgttactgtagagagaagtcagttgcttagtttcagcctcctgccggcaccagatgctgaccaggccctcccaggtgctgctgaactccttatctgtggaccctgatgccgagtgcaagtatggcctgtacttcagggacggccggcgcaaggtggactacatcctggtgtaccatcacaagaggccctcgggcaaccggaccctggtcaggagggtgcagcacagcgacaccccctctggggctcgcagcgtcaagcaggaccaccccctgccgggcaagggggcgtcgctggatgcaggctcgggggagcccccgatggactaccacgaggatgacaagcgcttccgcagggaggagtacgagggcaacctcctggaggcgggcctggagctggagcgggacgaggacactaaaatccacggagtcgggtttgtgaaaatccatgccccctggaacgtgctgtgcagagaggccgagtttctgaaactgaagatgccgacgaagaagatgtaccacattaatgagacccgtggcctcctgaaaaaaatcaactctgtgctccagaaaatcacagatcccatccagcccaaagtggctgagcacaggccccagaccatgaagagactctcctatcccttctcccgggagaagcagcatctatttgacttgtctgataaggattcctttttcgacagcaaaacccggagcacgattgtctatgagatcttgaagagaacgacgtgtacaaaggccaagtacagcatgggcatcacgagcctgctggccaatggtgtgtacgcggctgcatacccactgcacgatggagactacaacggtgaaaacgtcgagttcaacgacagaaaactcctgtacgaagagtgggcacgctatggagttttctataagtaccagcccatcgacctggtcaggaagtattttggggagaagatcggcctgtacttcgcctggctgggcgtgtacacccagatgctcatccctgcctccatcgtgggaatcattgtcttcctgtacggatgcgccaccatggatgaaaacatccccagcatggagatgtgtgaccagagacacaatatcaccatgtgcccgctttgcgacaagacctgcagctactggaagatgagctcagcctgcgccacggcccgcgccagccacctcttcgacaaccccgccacggtcttcttctctgtcttcatggccctctgggctgccaccttcatggagcactggaagcggaaacagatgcgactcaactaccgctgggacctcacgggctttgaagaggaagaggatcatcctagagctgaatacgaagccagagtcttggagaagtctctgaagaaagagtccagaaacaaagagactgacaaagtgaagctgacatggagagatcggttcccagcctacctcactaacttggtctccatcatcttcatgattgcagtgacgtttgccatcgtcctcggcgtcatcatctacaggatctccatggccgccgccttggccatgaactcctccccctccgtgcggtccaacatccgggtcacagtcacagccaccgcggtcatcatcaacctagtggtcatcatcctcctggacgaggtgtatggctgcatagcccgatggctcaccaagatcgaggtcccaaagacggagaaaagctttgaggagaggctgatcttcaaggctttcctgctgaagtttgtgaattcctacacccccatcttttacgtggcgttcttcaaaggccggtttgttggacgcccgggcgactatgtgtacattttccgttccttccgaatggaagaggtaaccgaaattttattcatctctggcagccccttctgtcttgcttacgatttgtctacaccatgcacttgggagaagcagctgcagcacatttgttcagcaaagagcagcaggtttctttccttcttgctggaaacttttctgttcccctgatgtggtggggtgggctttaatcttatctggtgtgttcatatagaatcacctagaaggataaagtcgctgtagagttaatgaaag

SEQ ID NO: 4 - DNA, 4558 bp, Artificial
  Homo sapiens mRNA
ggatggggagggcgcgccgcccggcggtcccagcgcacaggcggccacgatgagggtcaacgagaagtactcgacgctcccggccgaggaccgcagcgtccacatcatcaacatctgcgccatcgaggacatcggctacctgccgtccgagggcacgctgctgaactccttatctgtggaccctgatgccgagtgcaagtatggcctgtacttcagggacggccggcgcaaggtggactacatcctggtgtaccatcacaagaggccctcgggcaaccggaccctggtcaggagggtgcagcacagcgacaccccctctggggctcgcagcgtcaagcaggaccaccccctgccgggcaagggggcgtcgctggatgcaggctcgggggagcccccgatggactaccacgaggatgacaagcgcttccgcagggaggagtacgagggcaacctcctggaggcgggcctggagctggagcgggacgaggacactaaaatccacggagtcgggtttgtgaaaatccatgccccctggaacgtgctgtgcagagaggccgagtttctgaaactgaagatgccgacgaagaagatgtaccacattaatgagacccgtggcctcctgaaaaaaatcaactctgtgctccagaaaatcacagatcccatccagcccaaagtggctgagcacaggccccagaccatgaagagactctcctatcccttctcccgggagaagcagcatctatttgacttgtctgataaggattcctttttcgacagcaaaacccggagcacgattgtctatgagatcttgaagagaacgacgtgtacaaaggccaagtacagcatgggcatcacgagcctgctggccaatggtgtgtacgcggctgcatacccactgcacgatggagactacaacggtgaaaacgtcgagttcaacgacagaaaactcctgtacgaagagtgggcacgctatggagttttctataagtaccagcccatcgacctggtcaggaagtattttggggagaagatcggcctgtacttcgcctggctgggcgtgtacacccagatgctcatccctgcctccatcgtgggaatcattgtcttcctgtacggatgcgccaccatggatgaaaacatccccagcatggagatgtgtgaccagagacacaatatcaccatgtgcccgctttgcgacaagacctgcagctactggaagatgagctcagcctgcgccacggcccgcgccagccacctcttcgacaaccccgccacggtcttcttctctgtcttcatggccctctgggctgccaccttcatggagcactggaagcggaaacagatgcgactcaactaccgctgggacctcacgggctttgaagaggaagaggaggctgtcaaggatcatcctagagctgaatacgaagccagagtcttggagaagtctctgaagaaagagtccagaaacaaagagaagcgccggcatattccagaggagtcaacaaacaaatggaagcagagggttaagacagccatggcgggggtgaaattgactgacaaagtgaagctgacatggagagatcggttcccagcctacctcactaacttggtctccatcatcttcatgattgcagtgacgtttgccatcgtcctcggcgtcatcatctacagaatctccatggccgccgccttggccatgaactcctccccctccgtgcggtccaacatccgggtcacagtcacagccaccgcagtcatcatcaacctagtggtcatcatcctcctggacgaggtgtatggctgcatagcccgatggctcaccaagatcgaggtcccaaagacggagaaaagctttgaggagaggctgatcttcaaggctttcctgctgaagtttgtgaattcctacacccccatcttttacgtggcgttcttcaaaggccggtttgttggacgcccgggcgactacgtgtacattttccgttccttccgaatggaagagtgtgcgccagggggctgcctgatggagctatgcatccagctcagcatcatcatgctggggaaacagctgatccagaacaacctgttcgagatcggcatcccgaagatgaagaagctcatccgctacctgaagctgaagcagcagagcccccctgaccacgaggagtgtgtgaagaggaaacagcggtacgaggtggattacaacctggagcccttcgcgggcctcaccccagagtacatggaaatgatcatccagtttggcttcgtcaccctgtttgtcgcctccttccccctggccccactgtttgcgctgctgaacaacatcatcgagatccgcctggacgccaaaaagtttgtcactgagctccgaaggccggtagctgtcagagccaaagacatcggaatctggtacaatatcctcagaggcattgggaagcttgctgtcatcatcaatgccttcgtgatctccttcacgtctgacttcatcccgcgcctggtgtacctctacatgtacagtaagaacgggaccatgcacggcttcgtcaaccacaccctctcctccttcaacgtcagtgacttccagaacggcacggcccccaatgaccccctggacctgggctacgaggtgcagatctgcaggtataaagactaccgagagccgccgtggtcggaaaacaagtacgacatctccaaggacttctgggccgtcctggcagcccggctggcgtttgtcatcgtcttccagaacctggtcatgttcatgagcgactttgtggactgggtcatcccggacatccccaaggacatcagccagcagatccacaaggagaaggtgctcatggtggagctgttcatgcgggaggagcaagacaagcagcagctgctggaaacctggatggagaaggagcggcagaaggacgagccgccgtgcaaccaccacaacaccaaagcctgcccagacagcctcggcagcccagcccccagccatgcctaccacgggggcgtcctgtagctatgccagcggggctgggcaggccagccgggcatcctgaccgatgggcaccctctcccagggcaggcggcttcccgctcccaccagggcccggtgggtcctgggttttctgcaaacatggaggaccactttctgataggacattttcctttcttctttctgttttctttcccttgtttttgcacaaagccattatgcagggaatattttttaatctgtagtattcaagatgaatcaaaatgatggctggtaatacggcaataaggtagcaaaggcaggtgctttgcagaaagaatgcttggaaacttgagtctccctagaggtgaaaagtgagcagaggcccctagaaaccctcctctgaatcctcctaattccttaagatagatgcaaaatggtaagccgaggcatcgcgcaaaagctggtgcgatgcttcagggaaaatggaaaacccacgcaagaataatgattgattccggttccaaaaggtgtcacctacctgtttcagaaaagttagactttccatcgccttttccttccatcagttgagtggctgagagagaagtgcctcatccctgagccacacagggggcgtgggagcatcccagttatccctggaaagctagaaggggacagaggtgtccctgattaagcaggaaacagcacccttggcgtccccagcaggctccccactgtcagccacacacctgcccccatcacaccaagccgacctcagagttgttcatcttccttatgggacaaaaccggttgaccagaaaatgggcagagagagatgacctcggaagcatttccacagatggtgtcagggtttcaagaagtcttagggcttccaggggtcccctggaagctttagaatatttatgggtttttttttcaaatatcaattatgtggtagattgaggattttttttctgtagctcaaaggtggagggagtttattagttaaccaaatatcgttgagaggaatttaaaatactgttactaccaaagatttttattaataaaggcttatattttggtaacacttctctatatttttactcacaggaatgtcactgttggacaattattttaaaagtgtataaaaccaagtctcataaatgatatgagtgatctaaatttgcagcaatgatactaaacaactctctgaaatttctcaagcaccaagagaaacatcattttagcaaaggccaggaggaaaaatagaaataaatttgtcttgaagatctcattgatgtgatgttacattccctttaatctgccaactgtggtcaaagttcataggtgtcgtacatttccattatttgctaaaatcatgcaatctgatgcttctcttttctcttgtacagtaagtagtttgaagtgggttttgtatataaatactgtattaaaaattaggcaattaccaaaaatccttttatggaaaccatttttttaaaaagtgaatgtacacaaatccacagaggactgtggctggacattcatctaaataaatttgaatatacgaaaaaaaaaaaaaaaaaaaaaaaa

SEQ ID NO: 5 - DNA, 3432 bp, Artificial
  Homo sapiens transmembrane protein
atgggtctatgagatcttgaagagaacgacgtgtacaaaggccaagtacagcatgggcatcacgagcctgctggccaatggtgtgtacgcggctgcatacccactgcacgatggagactacaacggtgaaaacgtcgagttcaacgacagaaaactcctgtacgaagagtgggcacgctatggagttttctataagtaccagcccatcgacctggtcaggaagtattttggggagaagatcggcctgtacttcgcctggctgggcgtgtacacccagatgctcatccctgcctccatcgtgggaatcattgtcttcctgtacggatgcgccaccatggatgaaaacatccccagcatggagatgtgtgaccagagacacaatatcaccatgtgcccgctttgcgacaagacctgcagctactggaagatgagctcagcctgcgccacggcccgcgccagccacctcttcgacaaccccgccacggtcttcttctctgtcttcatggccctctgggctgccaccttcatggagcactggaagcggaaacagatgcgactcaactaccgctgggacctcacgggctttgaagaggaagaggaggctgtcaaggatcatcctagagctgaatacgaagccagagtcttggagaagtctctgaagaaagagtccagaaacaaagagactgacaaagtgaagctgacatggagagatcggttcccagcctacctcactaacttggtctccatcatcttcatgattgcagtgacgtttgccatcgtcctcggcgtcatcatctacagaatctccatggccgccgccttggccatgaactcctccccctccgtgcggtccaacatccgggtcacagtcacagccaccgcagtcatcatcaacctagtggtcatcatcctcctggacgaggtgtatggctgcatagcccgatggctcaccaagatcgaggtcccaaagacggagaaaagctttgaggagaggctgatcttcaaggctttcctgctgaagtttgtgaattcctacacccccatcttttacgtggcgttcttcaaaggccggtttgttggacgcccgggcgactacgtgtacattttccgttccttccgaatggaagagtgtgcgccagggggctgcctgatggagctatgcatccagctcagcatcatcatgctggggaaacagctgatccagaacaacctgttcgagatcggcatcccgaagatgaagaagctcatccgctacctgaagctgaagcagcagagcccccctgaccacgaggagtgtgtgaagaggaaacagcggtacgaggtggattacaacctggagcccttcgcgggcctcaccccagagtacatggaaatgatcatccagtttggcttcgtcaccctgtttgtcgcctccttccccctggccccactgtttgcgctgctgaacaacatcatcgagatccgcctggacgccaaaaagtttgtcactgagctccgaaggccggtagctgtcagagccaaagacatcggaatctggtacaatatcctcagaggcattgggaagcttgctgtcatcatcaatgccttcgtgatctccttcacgtctgacttcatcccgcgcctggtgtacctctacatgtacagtaagaacgggaccatgcacggcttcgtcaaccacaccctctcctccttcaacgtcagtgacttccagaacggcacggcccccaatgaccccctggacctgggctacgaggtgcagatctgcaggtataaagactaccgagagccgccgtggtcggaaaacaagtacgacatctccaaggacttctgggccgtcctggcagcccggctggcgtttgtcatcgtcttccagaacctggtcatgttcatgagcgactttgtggactgggtcatcccggacatccccaaggacatcagccaggagatccacaaggagaaggtgctcatggtggagctgttcatgcgggaggagcaagacaagcagcagctgctggaaacctggatggagaaggagcggcagaaggacgagccgccgtgcaaccaccacaacaccaaagcctgcccagacagcctcggcagcccagcccccagccatgcctaccacgggggcgtcctgtagctatgccagcggggctgggcaggccagccgggcatcctgaccgatgggcaccctctcccagggcaggcggcttcccgctcccaccagggcccggtgggtcctgggttttctgcaaacatggaggaccactttctgataggacattttcctttcttctttctgttttctttcccgtgtttttgcacaaagccattatgcagggaatattttttaatctgtagtattcaagatgaatcaaaatgatggctggtaatacggcaataaggtagcaaaggcaggtgctttgcagaaagaatgcttggaaacttgagtctccctagaggtgaaaggtgagcagaggcccctagaaaccctcctctgaatcctcctaattccttaagatagatgcaaaatggtaagccgaggcatcgcgcaaaagctggtgcgatgcttcagggaaaatggaaaacccacgcaagaataatgattgattccggttccaaaaggtgtcacctacctgtttcagaaaagttagactttccatcgccttttccttccatcagttgagtggctgagagagaagtgcctcatccctgagccacacagggggcgtgggagcatcccagttatccctggaaagctagaaggggacagaggtgtccctgattaagcaggaaacagcacccttggcgtccccagcaggctccccactgtcagccacacacctgcccccatcacaccaagtcgacctcagagttgttcatcttccttatgggacaaaaccggttgaccagaaaatgggcagagagagatgacctcggaagcatttccacagatggtgtcagggtttcaagaagtcttagggcttccaggggtcccctggaagctttagaatatttatgggtttttttttcaaatatcaattatatggtagattgaggattttttttctgtagctcaaaggtggagggagtttattagttaaccaaatatcgttgagaggaatttaaaatactgttactaccaaagatttttattaataaaggcttatattttggtaacacttctctatatttttactcacaggaatgtcactgttggacaattattttaaaagtgtataaaaccaagtctcataaatgatatgagtgatctaaatttgcagcaatgatactaaacaactctctgaaatttctcaagcaccaagagaaacatcattttagcaaaggccaggaggaaaaatagaaataaatttgtcttgaagatctcaaaaaaaaaaaaaaaaaaaaaaaaaaagaaaaaaaaaaaaaaaa

SEQ ID NO: 6 - DNA, 3272 bp, Artificial
  Homo sapiens transmembrane protein
agcggggccctgtgttactgtagagagaagtcagttgcttagtttcagcctcctgccggcaccaaatgctgaccaggccctcccaggtggtgagcaatgctgtttccaggagaggcatcctagggccagagaggccaaggtgccagggacatggccccaactgtcccctaaccaggggtagaggagtctagacccttgacatcaacacctatgaaagctgctgaactccttatctgtggaccctgatgccgagtgcaagtatggcctgtacttcagggacggccggcgcaaggtggactacatcctggtgtaccatcacaagaggccctcgggcaaccggaccctggtcaggagggtgcagcacagcgacaccccctctggggctcgcagcgtcaagcaggaccaccccctgccgggcaagggggcgtcgctggatgcaggctcaggggagcccccgatggactaccacgaggatgacaagcgcttccgcagggaggagtacgagggcaacctcctggaggcgggcctggagctggagcgggacgaggacactaaaatccacggagtcgggtttgtgaaaatccatgccccctggaacgtgctgtgcagagaggccgagtttctgaaactgaagatgccgacgaagaagatgtaccacattaatgagacccgtggcctcctgaaaaaaatcaactctgtgctccagaaaatcacagatcccatccagcccaaagtggctgagcacaggccccagaccatgaagagactctcctatcccttctcccgggagaagcagcatctatttgacttgtctgataaggattcctttttcgacagcaaaacccggagcacgattgtctatgagatcttgaagagaacgacgtgtacaaaggccaagtacagcatgggcatcacgagcctgctggccaatggtgtgtacgcggctgcatacccactgcacgatggagactacaacggtgaaaacgtcgagttcaacgacagaaaactcctgtacgaagagtgggcacgctatggagttttctataagtaccagcccatcgacctggtcaggaagtattttggggagaagatcggcctgtacttcgcctggctgggcgtgtacacccagatgctcatccctgcctccatcgtgggaatcattgtcttcctgtacggatgcgccaccatggatgaaaacatccccagcatggagatgtgtgaccagagacacaatatcaccatgtgcccgctttgcgacaagacctgcagctactggaagatgagctcagcctgcgccacggcccgcgccagccacctcttcgacaaccccgccacggtcttcttctctgtcttcatggccctctgggctgccaccttcatggagcactggaagcggaaacagatgcgactcaactaccgctgggacctcacgggctttgaagaggaagaggatcatcctagagctgaatacgaagcaagagtcttggagaagtctctgaagaaagagtctagaaacaaagagactgacaaagtgaagctgacatggagagatcggttcccagcctacctcactaacttggtctccatcatcttcatgattgcagtgacgtttgccatcgtcctcggcgtcatcatctacagaatctccatggccgccgccttggccatgaactcctccccctccgtgcggtccaacatccgggtcacagtcacagccaccgcagtcatcatcaacctagtggtcatcatcctcctggacgaggtgtatggctgcatagcccgatggctcaccaagatcgaggtcccaaagacggagaaaagctttgaggagaggctgatcttcaaggctttcctgctgaagtttgtgaattcctacacccccatcttttacgtggcgttcttcaaaggccggtttgttggacgcccgggcgactacgtgtacattttccgttccttccgaatggaagagtgtgcgccagggggctgcctgatggagctatgcatccagctcagcatcatcatgctggggaaacagctgatccagaacaacctgttcgagatcggcatcccgaagatgaagaagctcatccgctacctgaagctgaagcagcagagcccccctgaccacgaggagtgtgtgaagaggaaacagcggtacgaggtggattacaacctggagcccttcgcgggcctcaccccagagtacatggaaatgatcatccagtttggcttcgtcaccctgtttgtcgcctccttccccctggccccactgtttgcgctgctgaacaacatcatcgagatccgcctggacgccaaaaagtttgtcactgagctccgaaggccggtagctgtcagagccaaagacatcggaatctggtacaatatcctcagaggcattgggaagcttgctgtcatcatcgatgccttcgtgatctccttcacgtctgacttcatcccgcgcctggtgtacctctacatgtacagtaagaacgggaccatgcacggcttcgtcaaccacaccctctcctccttcaacgtcagtgacttccagaacggcacggcccccaatgaccccctggacctgggctacgaggtgcagatctgcaggtataaagactaccgagagccgccgtggtcggaaaacaagtacgacatctccaaggacttctgggccgtcctggcagcccggctggcgtttgtcatcgtcttccagaacctggtcatgttcatgagcgactttgtggactgggtcatcccggacatccccaaggacatcagccagcagatccacaaggagaaggtgctcatggtggagctgttcatgcgggaggagcaagacaagcagcagctgctggaaacctgtatggagaaggagcggcagaaggacgagccgccgtgcaaccaccacaacaccaaagcctgcccagacagcctcggcagcccagcccccagccatgcctaccacgggggcgtcctgtagctatgccagcggggctgggcaggccagccgggcatcctgaccgatgggcaccctctcccagggcaggcggcttcccgctcccaccagggcccggaatgtcactgttggacaattattttaaaagtgtataaaaccaagtctcataaatgatatgagtgatctaaatttgcagcaatgatactaaacaactctctgaaatttctcaagcaccaagagaaacatcattttagcaaaggccaggaggaaaaatagaaataaatttgtcttgaagatcccaaaaaaaaaaaaaaaa

SEQ ID NO: 7 - protein, 960 aa, Artificial
  Transmembrane protein
MRVNEKYSTLPAEDRSVHIINICAIEDIGYLPSEGTLLNSLSVDPDAECKYGLYFRDGRRKVDYILVYHHKRPSGNRTLVRRVQHSDTPSGARSVKQDHPLPGKGASLDAGSGEPPMDYHEDDKRFRREEYEGNLLEAGLELERDEDTKIHGVGFVKIHAPWNVLCREAEFLKLKMPTKKMYHINETRGLLKKINSVLQKITDPIQPKVAEHRPQTMKRLSYPFSREKQHLFDLSDKDSFFDSKTRSTIVYEILKRTTCTKAKYSMGITSLLANGVYAAAYPLHDGDYNGENVEFNDRKLLYEEWARYGVFYKYQPIDLVRKYFGEKIGLYFAWLGVYTQMLIPASIVGIIVFLYGCATMDENIPSMEMCDQRHNITMCPLCDKTCSYWKMSSACATARASHLFDNPATVFFSVFMALWAATFMEHWKRKQMRLNYRWDLTGFEEEEEAVKDHPRAEYEARVLEKSLKKESRNKETDKVKLTWRDRFPAYLTNLVSIIFMIAVTFAIVLGVIIYRISMAAALAMNSSPSVRSNIRVTVTATAVIINLVVIILLDEVYGCIARWLTKIEVPKTEKSFEERLIFKAFLLKFVNSYTPIFYVAFFKGRFVGRPGDYVYIFRSFRMEECAPGGCLMELCIQLSIIMLGKQLIQNNLFEIGIPKMKKLIRYLKLKQQSPPDHEECVKRKQRYEVDYNLEPFAGLTPEYMEMIIQFGFVTLFVASFPLAPLFALLNNIIEIRLDAKKFVTELRRPVAVRAKDIGIWYNILRGIGKLAVIINAFVISFTSDFIPRLVYLYMYSKNGTMHGFVNHTLSSFNVSDFQNGTAPNDPLDLGYEVQICRYKDYREPPWSENKYDISKDFWAVLAARLAFVIVFQNLVMFMSDFVDWVIPDIPKDISQQIHKEKVLMVELFMREEQDKQQLLETWMEKERQKDEPPCNHHNTKACPDSLGSPAPSHAYHGGVL

SEQ ID NO: 8 - protein, 594 aa, Artificial
  Homo sapiens
MEMCDQRHNITMCPLCDKTCSYWKMSSACATARASHLFDNPATVFFSVFMALWAATFMEHWKRKQMRLNYRWDLTGFEEEEEAVKDHPRAEYEARVLEKSLKKESRNKETDKVKLTWRDRFPAYLTNLVSIIFMIAVTFAIVLGVIIYRISMAAALAMNSSPSVRSNIRVTVTATAVIINLVVIILLDEVYGCIARWLTKIEVPKTEKSFEERLIFKAFLLKFVNSYTPIFYVAFFKGRFVGRPGDYVYIFRSFRMEECAPGGCLMELCIQLSIIMLGKQLIQNNLFEIGIPKMKKLIRYLKLKQQSPPDHEECVKRKQRYEVDYNLEPFAGLTPEYMEMIIQFGFVTLFVASFPLAPLFALLNNIIEIRLDAKKFVTELRRPVAVRAKDIGIWYNILRGIGKLAVIINAFVISFTSDFIPRLVYLYMYSKNGTMHGFVNHTLSSFNVSDFQNGTAPNDPLDLGYEVQICRYKDYREPPWSENKYDISKDFWAVLAARLAFVIVFQNLVMFMSDFVDWVIPDIPKDISQQIHKEKVLMVELFMREEQDKQQLLETWMEKERQKDEPPCNHHNTKACPDSLGSPAPSHAYHGGVL

SEQ ID NO: 9 - protein, 642 aa, Artificial
  Homo sapiens
MLTRPSQVLLNSLSVDPDAECKYGLYFRDGRRKVDYILVYHHKRPSGNRTLVRRVQHSDTPSGARSVKQDHPLPGKGASLDAGSGEPPMDYHEDDKRFRREEYEGNLLEAGLELERDEDTKIHGVGFVKIHAPWNVLCREAEFLKLKMPTKKMYHINETRGLLKKINSVLQKITDPIQPKVAEHRPQTMKRLSYPFSREKQHLFDLSDKDSFFDSKTRSTIVYEILKRTTCTKAKYSMGITSLLANGVYAAAYPLHDGDYNGENVEFNDRKLLYEEWARYGVFYKYQPIDLVRKYFGEKIGLYFAWLGVYTQMLIPASIVGIIVFLYGCATMDENIPSMEMCDQRHNITMCPLCDKTCSYWKMSSACATARASHLFDNPATVFFSVFMALWAATFMEHWKRKQMRLNYRWDLTGFEEEEDHPRAEYEARVLEKSLKKESRNKETDKVKLTWRDRFPAYLTNLVSIIFMIAVTFAIVLGVIIYRISMAAALAMNSSPSVRSNIRVTVTATAVIINLVVIILLDEVYGCIARWLTKIEVPKTEKSFEERLIFKAFLLKFVNSYTPIFYVAFFKGRFVGRPGDYVYIFRSFRMEEVTEILFISGSPFCLAYDLSTPCTWEKQLQHICSAKSSRFLSFLLETFLFP

SEQ ID NO: 10 - protein, 712 aa, Artificial
  Homo sapiens
WVYEILKRTTCTKAKYSMGITSLLANGVYAAAYPLHDGDYNGENVEFNDRKLLYEEWARYGVFYKYQPIDLVRKYFGEKIGLYFAWLGVYTQMLIPASIVGIIVFLYGCATMDENIPSMEMCDQRHNITMCPLCDKTCSYWKMSSACATARASHLFDNPATVFFSVFMALWAATFMEHWKRKQMRLNYRWDLTGFEEEEEAVKDHPRAEYEARVLEKSLKKESRNKETDKVKLTWRDRFPAYLTNLVSIIFMIAVTFAIVLGVIIYRISMAAALAMNSSPSVRSNIRVTVTATAVIINLVVIILLDEVYGCIARWLTKIEVPKTEKSFEERLIFKAFLLKFVNSYTPIFYVAFFKGRFVGRPGDYVYIFRSFRMEECAPGGCLMELCIQLSIIMLGKQLIQNNLFEIGIPKMKKLIRYLKLKQQSPPDHEECVKRKQRYEVDYNLEPFAGLTPEYMEMIIQFGFVTLFVASFPLAPLFALLNNIIEIRLDAKKFVTELRRPVAVRAKDIGIWYNILRGIGKLAVIINAFVISFTSDFIPRLVYLYMYSKNGTMHGFVNHTLSSFNVSDFQNGTAPNDPLDLGYEVQICRYKDYREPPWSENKYDISKDFWAVLAARLAFVIVFQNLVMFMSDFVDWVIPDIPKDISQEIHKEKVLMVELFMREEQDKQQLLETWMEKERQKDEPPCNHHNTKACPDSLGSPAPSHAYHGGVL

SEQ ID NO: 11 - protein, 840 aa, Artificial
  Homo sapiens
MDYHEDDKRFRREEYEGNLLEAGLELERDEDTKIHGVGFVKIHAPWNVLCREAEFLKLKMPTKKMYHINETRGLLKKINSVLQKITDPIQPKVAEHRPQTMKRLSYPFSREKQHLFDLSDKDSFFDSKTRSTIVYEILKRTTCTKAKYSMGITSLLANGVYAAAYPLHDGDYNGENVEFNDRKLLYEEWARYGVFYKYQPIDLVRKYFGEKIGLYFAWLGVYTQMLIPASIVGIIVFLYGCATMDENIPSMEMCDQRHNITMCPLCDKTCSYWKMSSACATARASHLFDNPATVFFSVFMALWAATFMEHWKRKQMRLNYRWDLTGFEEEEDHPRAEYEARVLEKSLKKESRNKETDKVKLTWRDRFPAYLTNLVSIIFMIAVTFAIVLGVIIYRISMAAALAMNSSPSVRSNIRVTVTATAVIINLVVIILLDEVYGCIARWLTKIEVPKTEKSFEERLIFKAFLLKFVNSYTPIFYVAFFKGRFVGRPGDYVYIFRSFRMEECAPGGCLMELCIQLSIIMLGKQLIQNNLFEIGIPKMKKLIRYLKLKQQSPPDHEECVKRKQRYEVDYNLEPFAGLTPEYMEMIIQFGFVTLFVASFPLAPLFALLNNIIEIRLDAKKFVTELRRPVAVRAKDIGIWYNILRGIGKLAVIIDAFVISFTSDFIPRLVYLYMYSKNGTMHGFVNHTLSSFNVSDFQNGTAPNDPLDLGYEVQICRYKDYREPPWSENKYDISKDFWAVLAARLAFVIVFQNLVMFMSDFVDWVIPDIPKDISQQIHKEKVLMVELFMREEQDKQQLLETCMEKERQKDEPPCNHHNTKACPDSLGSPAPSHAYHGGVL

SEQ ID NO: 12 - DNA, 5084 bp, Artificial
  KIT nucleotide sequence
gatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttgccatactttgtctgaaaaattcctttgtgtttctattgacttcaatgatagtaagaaaagtggttgttagttatagatgtctaggtacttcaggggcacttcattgagagttttgtcaatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattc

SEQ ID NO: 13 - DNA, 5179 bp, Artificial
  KIT nucleotide sequence
ggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccatagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacatgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatttagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaactaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaaaaaaaaaaaaaaaa

SEQ ID NO: 14 - DNA, 5084 bp, Artificial; identical to SEQ ID NO: 12
  KIT nucleotide sequence

SEQ ID NO: 15 - protein, 976 aa, Artificial [sequence omitted]

SEQ ID NO: 16 - protein, 976 aa, Artificial [sequence omitted]

SEQ ID NO: 17 - protein, 972 aa, Artificial
  KIT amino acid sequence
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 18 - protein, 976 aa, Artificial [sequence omitted]

SEQ ID NO: 19 - protein, 976 aa, Artificial [sequence omitted]

SEQ ID NO: 20 - protein, 976 aa, Artificial [sequence omitted]

SEQ ID NO: 21 - protein, 976 aa, Artificial [sequence omitted]

SEQ ID NO: 22 - DNA, 1176 bp, Artificial
  PDGFRA nucleotide sequence
tgcaagtgttattcgacaaaagcaattatgctaatttccttccctgtggctcaattccttttttgacacgatgacttggaggagtcattatgattactccaaacaggaaagacactcgcccagctgtccgcccgcagagagctggctacggtgcagaaagctgaggaggcgtctggagtttttgggtgttaatgattctgcctgcccacaggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttaggctgggctggtggagttggcacgagatgtcagaggaacctgagtcatgctcaggcccaagccctgttggcaggcagaccactgctttctggccttccgtgactatctgaaaaaaatcgtgaatggctagagctactcttcacttgctgaacattttcaaaaagaattgacacttgctgaacatttcttttcaattcagaacttctgatggattaaattgccttcttcctcgaaaaccctgggacccttccagatgggactaactggggaaagtggacaagttacaaacaaagaaactcaaaggaaagtcattggcactgatctctaagatgctatcacatgtgattggtggttgattttattaacaaattataagcaaagtactacaaaggtggctttaaaaagaaaataaagcaattcacagaaact

SEQ ID NO: 23 - DNA, 6378 bp, Artificial
  PDGFRA nucleotide sequence
cccattactgttggagctacagggagagaaacaggaggagactgcaagagatcatttgggaaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccggatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgtcctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggacgacatcggcatagactcttcagacctggtggaagacagcttcctgtaactggcggattcgaggggttccttccacttctggggccacctctggatcccgttcagaaaaccactttattgcaatgcggaggttgagaggaggacttggttgatgtttaaagagaagttcccagccaagggcctcggggagcgttctaaatatgaatgaatgggatattttgaaatgaactttgtcagtgttgcctctcgcaatgcctcagtagcatctcagtggtgtgtgaagtttggagatagatggataagggaataataggccacagaaggtgaactttgtgcttcaaggacattggtgagagtccaacagacacaatttatactgcgacagaacttcagcattgtaattatgtaaataactctaaccaaggctgtgtttagattgtattaactatcttctttggacttctgaagagaccactcaatccatccatgtacttccctcttgaaacctgatgtcagctgctgttgaactttttaaagaagtgcatgaaaaaccatttttgaaccttaaaaggtactggtactatagcattttgctatcttttttagtgttaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccagagaaagcacaatttaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcaaaaagactggatttgcagaagttttttttttttttcttcatgcctgatgaaagctttggcaaccccaatatatgtattttttgaatctatgaacctgaaaagggtcagaaggatgcccagacatcagcctccttctttcaccccttaccccaaagagaaagagtttgaaactcgagaccataaagatattctttagtggaggctggatgtgcattagcctggatcctcagttctcaaatgtgtgtggcagccaggatgactagatcctgggtttccatccttgagattctgaagtatgaagtctgagggaaaccagagtctgtatttttctaaactccctggctgttctgatcggccagttttcggaaacactgacttaggtttcaggaagttgccatgggaaacaaataatttgaactttggaacagggttggaattcaaccacgcaggaagcctactatttaaatccttggcttcaggttagtgacatttaatgccatctagctagcaattgcgaccttaatttaactttccagtcttagctgaggctgagaaagctaaagtttggttttgacaggttttccaaaagtaaagatgctacttcccactgtatgggggagattgaactttccccgtctcccgtcttctgcctcccactccataccccgccaaggaaaggcatgtacaaaaattatgcaattcagtgttccaagtctctgtgtaaccagctcagtgttttggtggaaaaaacattttaagttttactgataatttgaggttagatgggaggatgaattgtcacatctatccacactgtcaaacaggttggtgtgggttcattggcattctttgcaatactgcttaattgctgataccatatgaatgaaacatgggctgtgattactgcaatcactgtgctatcggcagatgatgctttggaagatgcagaagcaataataaagtacttgactacctactggtgtaatctcaatgcaagccccaactttcttatccaactttttcatagtaagtgcgaagactgagccagattggccaattaaaaacgaaaacctgactaggttctgtagagccaattagacttgaaatacgtttgtgtttctagaatcacagctcaagcattctgtttatcgctcactctcccttgtacagccttattttgttggtgctttgcattttgatattgctgtgagccttgcatgacatcatgaggccggatgaaacttctcagtccagcagtttccagtcctaacaaatgctcccacctgaatttgtatatgactgcatttgtgggtgtgtgtgtgttttcagcaaattccagatttgtttccttttggcctcctgcaaagtctccagaagaaaatttgccaatctttcctactttctatttttatgatgacaatcaaagccggcctgagaaacactatttgtgactttttaaacgattagtgatgtccttaaaatgtggtctgccaatctgtacaaaatggtcctatttttgtgaagagggacataagataaaatgatgttatacatcaatatgtatatatgtatttctatatagacttggagaatactgccaaaacatttatgacaagctgtatcactgccttcgtttatatttttttaactgtgataatccccacaggcacattaactgttgcacttttgaatgtccaaaatttatattttagaaataataaaaagaaagatacttacatgttcccaaaacaatggtgtggtgaatgtgtgagaaaaactaacttgatagggtctaccaatacaaaatgtattacgaatgcccctgttcatgtttttgttttaaaacgtgtaaatgaagatctttatatttcaataaatgatatataatttaaagtt

SEQ ID NO: 24 - DNA, 6375 bp, Artificial
  PDGFRA nucleotide sequence
ttggagctacagggagagaaacagaggaggagactgcaagagatcattggaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttagactcagaagtcaaaaacctcctttcagatgataactcagaaggccttactttattggatttgttgagcttcacctatcaagttgcccgaggaatggagtttttggcttcaaaaaattgtgtccaccgtgatctggctgctcgcaacgttctcctggcacaaggaaaaattgtgaagatctgtgactttggcctggccagagacatcatgcatgattcgaactatgtgtcgaaaggcagtacctttctgcccgtgaagtggatggctcctgagagcatctttgacaacctctacaccacactgagtgatgtctggtcttatggcattctgctctgggagatcttttcccttggtggcaccccttaccccggcatgatggtggattctactttctacaataagatcaagagtgggtaccggatggccaagcctgaccacgctaccagtgaagtctacgagatcatggtgaaatgctggaacagtgagccggagaagagaccctccttttaccacctgagtgagattgtggagaatctgctgcctggacaatataaaaagagttatgaaaaaattcacctggacttcctgaagagtgaccatcctgctgtggcacgcatgcgtgtggactcagacaatgcatacattggtgtcacctacaaaaacgaggaagacaagctgaaggactgggagggtggtctggatgagcagagactgagcgctgacagtggctacatcattcctctgcctgacattgaccctgtccctgaggaggaggacctgggcaagaggaacagacacagctcgcagacctctgaagagagtgccattgagacgggttccagcagttccaccttcatcaagagagaggacgagaccattgaagacatcgacatgatggacgacatcggcatagactcttcagacctggtggaagacagcttcctgtaactggcggattcgaggggttccttccacttctggggccacctctggatcccgttcagaaaaccactttattgcaatgcggaggttgagaggaggacttggttgatgtttaaagagaagttcccagccaagggcctcggggagcctttctaaatatgaatgaatgggatattttgaaatgaactttgtcagtgttgcctcttgcaatgcctcagtagcatctcagtggtgtgtgaagtttggagatagatggataagggaataataggccacagaaggtgaactttctgcttcaaggacattggtgagagtccaacagacacaatttatactgcgacagaacttcagcattgtaattatgtaaataactctaaccacggctgtgtttagattgtattaactatcttctttggacttctgaagagaccactcaatccatccatgtacttccctcttgaaacctgatgtcagctgctgttgaactttttaaagaagtgcatgaaaaaccatttttgaccttaaaaggtactggtactatagcattttgctatcttttttagtgttaaagagataaagaataataattaaccaaccttgtttaatagatttgggtcatttagaagcctgacaactcattttcatattgtaatctatgtttataatactactactgttatcagtaatgctaaatgtgtaataatgtaacatgatttccctccacacaaagcacaatttaaaaacaatccttactaagtaggtgatgagtttgacagtttttgacatttatattaaataacatgtttctctataaagtatggtaatagctttagtgaattaaatttagttgagcatagagaacaaagtaaaagtagtgttgtccaggaagtcagaatttttaactgtactgaataggttccccaatccatcgtattaaaaaacaattaactgccctctgaaataatgggattagaaacaaacaaaactcttaagtcctaaaagttctcaatgtagaggcataaacctgtgctgaacataacttctcatgtatattacccaatggaaaatataatgatcagcgcanaaagactggatttgcagaagttnttttttttttttcttcttgcctgatgaaagctttggcgaccccaatatatgtattttttgaatctatgaacctgaaaagggtcacaaaggatgcccagacatcagcctccttctttcaccccttaccccaaagagaaagagtttgaaactcgagaccataaagatattctttagtggaggctggaagtgcattagcctgatcctcagttctcaaatgtgtgtggcagccaggtagactagtacctgggtttccatccttgagattctgaagtatgaagtctgagggaaaccagagtctgtatttttctaaactccctggctgttctgatcggccaggtttcggaaacactgacttaggtttcaggaagttgccatgggaaacaaataatttgaactttggaacagggttcttaagttggtgcgtccttcggatgataaatttaggaaccgaagtccaatcactgtaaattacggtagatcgatcgttaacgctggaattaaattgaaaggtcagaatcgactccgactctttcgatttcaaaccaaaactgtccaaaaggttttcatttctacgatgaagggtgacataccccctctaacttgaaaggggcagagggcagaagagcggagggtgaggtatggggcggttcctttccgtacatgtttttaatacgttaagtcacaaggttcagagacacattggtcgagtcacaaaaccaccttttttgtaaaattcaaaatgactattaaactccaatctaccctcctacttaacagtgtagataggtgtgacagtttgtccaaccacacccaagtaaccgtaagaaacgttatgacgaattaacgactatggtatacttactttgtacccgacactaatgacgttagtgacacgatagccgtctactacgaaaccttctacgtcttcgttattatttcatgaactgatggatgaccacattagagttacgttcggggttgaaagaataggttgaaaaagtatcattcacgcttctgactcggtctaaccggttaatttttcttttggactgatccaagacatctcggttaatctgaactttatgcaaacacaaagatcttagtgtcgagttcgtaagacaaatagcgagtgagagggaacatgtcggaataaaacaaccacgaaacgtaaaactataacgacactcggaacgtactgtagtactccggcctactttgaagagtcaggtcgtcaaaggtcaggattgtttacgagggtggacttaaacatatactgacgtaaacacccacacacacacaaaagtcgtttaaggtctaaacaaaggaaaaccggaggacgtttcagaggtcttcttttaaacggttagaaaggatgaaagataaaaatactactgttagtttcggccggactctttgtgataaacactgaaaaatttgctaatcactacaggaattttacaccagacggttagacatgttttaccaggataaaaacacttctccctgtattctattttactacaatatgtagttatacatatatacataaagatatatctgaacctcttatgacggttttgtaaatactgttcgacatagtgacggaagcaaatataaaaaaattgacactattaggggtgtccgtgtaattgacaacgtgaaaacttacaggttttaaatataaaatctttattatttttctttctatgaatgtacaagggttttgttaccacaccacttacacactctttttgattgaactatcccagatggttatgttttacataatgcttacggggacaagtacaaaaacaaaattttgcacatttacttctagaaatataaagttatttactatatattaaatttccttaag

SEQ ID NO: 25 - DNA, 1176 bp, Artificial; identical to SEQ ID NO: 22
  PDGFRA nucleotide sequence

SEQ ID NO: 26 - protein, 1089 aa, Artificial
  PDGFRA amino acid sequence
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 27 - protein, 1089 aa, Artificial; identical to SEQ ID NO: 26
  PDGFRA amino acid sequence

SEQ ID NO: 28 - protein, 1089 aa, Artificial; identical to SEQ ID NO: 26
  PDGFRA amino acid sequence

SEQ ID NO: 29 - protein, 1089 aa, Artificial; identical to SEQ ID NO: 26
  PDGFRA amino acid sequence

(19) United States
(12) Patent Application Publication
(10) Pub. No.: US 2006/0040292 A1
(43) Pub. Date: Feb. 23, 2006

(54) Tumor markers and uses thereof

(76) Inventors: Robert B. West, Menlo Park, CA (US); Matthijs van de Rijn, La Honda, CA (US)

Correspondence Address: CHOATE, HALL & STEWART;Attn: Brenda Herschbach Jarrell, Ph.D., 53 State Street, Exchange Place, Boston, MA, 02109, US

(21) Appl. No.: 11/177,894

(22) Filed: Jul. 8, 2005

Related U.S. Application Data
(60) Provisional application No. 60/586,676, filed on Jul. 8, 2004.

(51) Int. Cl. C12Q 1/68; G01N 33/574

(52) U.S. Cl. 435/6; 435/7.23

(57) ABSTRACT

The present invention relates to three gene markers that are useful in classifying tumors. The markers are known herein as DOG1 (GenBank GeneID 55107), KIT (GenBank GeneID 3815) and PDGFRA (GenBank GeneID 5156) and can be used alone or in combination to identify subclasses of tumors. The classification methods are exemplified herein using gastrointestinal stromal tumors (GISTs). It is to be understood and will be appreciated that the same markers may be used in the classification of tumors other than GISTs. The methods may further comprise providing diagnostic, prognostic, or predictive information based on the classifying step. Classifying may include stratifying the tumor (and thus stratifying a subject having the tumor), e.g., for a clinical trial. The methods may further comprise selecting a treatment based on the classifying step.

### PRIORITY INFORMATION

This application claims priority to U.S. Ser. No. 60/586,676 filed Jul. 8, 2004. The entire contents of this priority application is hereby incorporated by reference.

### GOVERNMENT SUPPORT

The U.S. Government has a paid-up license in this invention and the right in limited circumstances to require the patent owner to license others on reasonable terms as provided for by the terms of Grant No. NIH CA85129 and CA84967.

### BACKGROUND OF THE INVENTION

A major challenge of cancer treatment is to select specific therapies for distinct tumor types in order to maximize efficacy and minimize toxicity. A related challenge lies in the attempt to provide accurate diagnostic, prognostic, and predictive information. At present, tumors are primarily classified under the tumor-node-metastasis (TNM) system. This system, which uses the size of the tumor, the presence or absence of tumor in regional lymph nodes, and the presence or absence of distant metastases, to assign a stage to the tumor is described in the American Joint Committee on Cancer: AJCC Cancer Staging Manual. Philadelphia, Pa.: Lippincott-Raven Publishers, 5th ed., 1997, pp 171-180, and Harris, JR: “Staging of breast carcinoma” in Harris, J R, Hellman, S, Henderson, I C, Kinne D W (eds.): Breast Diseases. Philadelphia, Lippincott, 1991. The assigned stage is used as a basis for selection of appropriate therapy and for prognostic purposes. In addition to the TNM parameters, morphologic appearance is used to further classify tumors and thereby aid in selection of appropriate therapy. However, this approach has serious limitations. Tumors with similar histopathologic appearance can exhibit significant variability in terms of clinical course and response to therapy. For example, some tumors are rapidly progressive while others are not. Some tumors respond readily to hormonal therapy or chemotherapy while others are resistant.

Assays for cell surface tumor markers, e.g., using immunohistochemistry, have recently provided means for dividing certain tumor types into subclasses. For example, one factor considered in prognosis and treatment decisions for breast cancer is the presence or absence of the estrogen receptor (ER) in tumor samples. ER-positive breast cancers typically respond much more readily to hormonal therapies such as tamoxifen, which acts as an anti-estrogen in breast tissue, than ER-negative tumors.

Although a substantial number of genes have been implicated as playing important roles in cancer, the factors responsible for the phenotypic diversity of tumors remain largely unknown. In particular, understanding of the underlying differences in gene expression and mutation that may contribute to tumor phenotype is limited. Understanding the differences between normal and cancerous tissue and between different tumors of the same tissue type is of significant diagnostic, prognostic, and therapeutic utility. There is therefore a need for the identification of genes and proteins exhibiting different mutations between or among tumors. There is also a need for the identification of genes and proteins exhibiting different levels of levels of expression and/or activity between or among tumors. In particular, there is a need for the identification of additional genes and proteins that can be used to classify tumors, especially genes and proteins that can provide diagnostic, prognostic, and/or predictive information in cancer. There is also a need for binding agents and other reagents for the detection and measurement of such genes and proteins.

### SUMMARY OF THE INVENTION

The present invention relates to three gene markers that are useful in classifying tumors. The markers are known herein as DOG1(GenBank GeneID 55107), KIT (GenBank GeneID 3815) and PDGFRA (GenBank GeneID 5156) and can be used alone or in combination to identify subclasses of tumors. The classification methods are exemplified herein using gastrointestinal stromal tumors (GISTs). It is to be understood and will be appreciated that some or all of the same markers may be used in the classification of tumors other than GISTs. The methods may further comprise providing diagnostic, prognostic, or predictive information based on the classifying step. For example, this may involve stratifying the tumor (and thus stratifying a subject having the tumor) for a clinical trial. The methods may further comprise selecting a treatment based on the classifying step.

### BRIEF DESCRIPTION OF THE DRAWING

FIG. 1 shows gene array measurements of KIT and DOG1 mRNA expression in 30 soft tissue tumors. Red indicates a relatively high level of expression while green denotes a low level of expression. Gene array data for soft tissue tumors (STTs) 524, 629, 417, 418, 219, 111, 656, 94, 335, 794, 1148, 850, 616, 710, 523, 526, 740, 607, and 1220 are also described by the inventors in Nielsen et al., “Molecular characterisation of soft tissue tumours: a gene expression study”, Lancet 359:1301-1307, 2002.

FIG. 2 shows hierarchical clustering of the KIT IHC, KIT ISH, PDGFRA ISH, DOG1 IHC, and DOG1 ISH data (IHC=immunohistochemical staining, ISH=in situ hybridization). The results for GISTs on the two TMAs described in the Examples have been combined. Antisera or hybridization probes are in columns, tumors in rows. Bright red denotes strong reactivity, while dark red and green indicate low and absent reactivity, respectively. White means missing data.

FIG. 3 shows the staining results on GISTs for KIT IHC, KIT ISH, PDGFRA ISH, DOG1 IHC, and DOG1 ISH in graphic form (see also Table 1).

FIG. 4 shows immunohistochemical staining results with anti-DOG1 serum (S284) and anti-KIT serum on two GISTs (TMA 822 (A) and 3688 (B)) and a synovial sarcoma (TMA 856 (C)).

FIG. 5 shows in situ hybridization results of a GIST and leiomyosarcoma with antisense probes to DOG1 and KIT on a GIST and a leiomyosarcoma (LMS). The corresponding negative control sense probes are included in the inset in the upper right hand corner of the GIST sample.

FIG. 6 shows in situ hybridization results for KIT, DOG1, and PDGFRA with GISTs. (A) KIT positive mutant GIST shows positive ISH for KIT and DOG1 but not PDGFRA. (B) PDGFRA positive mutant GIST shows positive ISH for DOG1 and PDGFRA but not for KIT. (C) Negative control leiomyosarcoma.

FIG. 7 lists some of the DOG1 nucleotide sequences that have been deposited with GenBank as of Jul. 4, 2004 (see GenBank GeneID 55107):
SEQ ID NO:1 corresponds to GenBank accession number NM_{—}018043.
SEQ ID NO:2 corresponds to GenBank accession number AK001123.
SEQ ID NO:3 corresponds to GenBank accession number AK097619.
SEQ ID NO:4 corresponds to GenBank accession number AL833582.
SEQ ID NO:5 corresponds to GenBank accession number BC027590.
SEQ ID NO:6 corresponds to GenBank accession number BC033036.

FIG. 8 lists some of the DOG1 amino acid sequences that have been deposited with GenBank as of Jul. 4, 2004 (see GenBank GeneID 55107):
SEQ ID NO:7 corresponds to GenBank accession number NP_{—}060513.
SEQ ID NO:8 corresponds to GenBank accession number BAA91513.
SEQ ID NO:9 corresponds to GenBank accession number BAC05123.
SEQ ID NO:10 corresponds to GenBank accession number AAH27590.
SEQ ID NO:11 corresponds to GenBank accession number AAH33036.

FIG. 9 lists some of the KIT nucleotide sequences that have been deposited with GenBank as of Jul. 4, 2004 (see GenBank GeneID 3815):
SEQ ID NO:12 corresponds to GenBank accession number NM_{—}000222.
SEQ ID NO:13 corresponds to GenBank accession number BC071593.
SEQ ID NO:14 corresponds to GenBank accession number X06182.

FIG. 10 lists some of the KIT amino acid sequences that have been deposited with GenBank as of Jul. 4, 2004 (see GenBank GeneID 3815):
SEQ ID NO:15 corresponds to GenBank accession number NP_{—}000213.
SEQ ID NO:16 corresponds to GenBank accession number AAC50968.
SEQ ID NO:17 corresponds to GenBank accession number AAC50969.
SEQ ID NO:18 corresponds to GenBank accession number CAA49159.
SEQ ID NO:19 corresponds to GenBank accession number AAH71593.
SEQ ID NO:20 corresponds to GenBank accession number CAA29548.
SEQ ID NO:21 corresponds to GenBank accession number P10721.

FIG. 11 lists some of the PDGFRA nucleotide sequences that have been deposited with GenBank as of Jul. 4, 2004 (see GenBank GeneID 5156):
SEQ ID NO:22 corresponds to GenBank accession number L25829.
SEQ ID NO:23 corresponds to GenBank accession number M21574.
SEQ ID NO:24 corresponds to GenBank accession number M22734.
SEQ ID NO:25 corresponds to GenBank accession number X76079.

FIG. 12 lists some of the PDGFRA amino acid sequences that have been deposited with GenBank as of Jul. 4, 2004 (see GenBank GeneID 5156):
SEQ ID NO:26 corresponds to GenBank accession number BAA08742.
SEQ ID NO:27 corresponds to GenBank accession number AAA96715.
SEQ ID NO:28 corresponds to GenBank accession number AAA60048.
SEQ ID NO:29 corresponds to GenBank accession number P16234.

FIG. 13 shows ClustalW alignments of DOG1 nucleotide sequences of FIG. 7.

FIG. 14 shows ClustalW alignments of DOG1 polypeptide sequences of FIG. 8.

FIG. 15 shows ClustalW alignments of KIT nucleotide sequences of FIG. 9.

FIG. 16 shows ClustalW alignments of KIT polypeptide sequences of FIG. 10.

FIG. 17 shows ClustalW alignments of PDGFRA nucleotide sequences of FIG. 11.

FIG. 18 shows ClustalW alignments of PDGFRA polypeptide sequences of FIG. 12.

### DETAILED DESCRIPTION OF CERTAIN PREFERRED EMBODIMENTS

The present application refers to various patents, publications, books, articles, and other references. The contents of all of these items are hereby incorporated by reference in their entirety. In particular, a numbered list of references appears following the Examples, all of which are incorporated herein by reference.

I. Definitions

To facilitate understanding of the invention, the following definitions are provided. It is to be understood that, in general, terms not otherwise defined are to be given their meaning or meanings as generally accepted in the art.

DOG1 polypeptide: The DOG1 gene encodes a transmembrane protein of unknown function (officially named TMEM16A or transmembrane protein 16A). Sequence analysis predicts the presence of eight transmembrane spanning segments. DOG1 has been recently mapped to 11q13.2 on chromosome 11 ²⁶. The GenBank GeneID for DOG1 is 55107. FIG. 8 lists some of the DOG1 amino acid sequences that have been deposited with GenBank under GeneID 55107 as of Jul. 4, 2004. SEQ ID NO:7 corresponds to GenBank accession number NP_{—}060513; SEQ ID NO:8 corresponds to GenBank accession number BAA91513; SEQ ID NO:9 corresponds to GenBank accession number BAC05123; SEQ ID NO:10 corresponds to GenBank accession number AAH27590; and SEQ ID NO:11 corresponds to GenBank accession number AAH33036. FIG. 14 shows ClustalW alignments of these DOG1 amino acid sequences. As used herein a “DOG1 polypeptide” is a polypeptide that comprises the amino acid sequence of SEQ ID NO:7, 8, 9, 10, 11, a fragment thereof, or an allelic variant thereof.

KIT polypeptide: The KIT gene encodes the human homolog of the proto-oncogene c-kit. KIT is a three transmembrane receptor for mast cell growth factor (also known as stem cell factor). The KIT gene maps to 4q11-q12 on chromosome 4. The GenBank GeneID for KIT is 3815. FIG. 10 lists some of the KIT nucleotide sequences that have been deposited with GenBank under GeneID 3815 as of Jul. 4, 2004. SEQ ID NO:15 corresponds to GenBank accession number NP_{—}000213; SEQ ID NO:16 corresponds to GenBank accession number AAC50968; SEQ ID NO:17 corresponds to GenBank accession number AAC50969; SEQ ID NO:18 corresponds to GenBank accession number CAA49159; SEQ ID NO:19 corresponds to GenBank accession number AAH71593; SEQ ID NO:20 corresponds to GenBank accession number CAA29548; and SEQ ID NO:21 corresponds to GenBank accession number P10721. FIG. 16 shows ClustalW alignments of these KIT amino acid sequences. As used herein a “KIT polypeptide” is a polypeptide that comprises the amino acid sequence of SEQ ID NO:15, 16, 17, 18, 19, 20, 21, a fragment thereof or an allelic variant thereof.

PDGFRA polypeptide: The PDGFRA gene encodes a cell surface tyrosine kinase receptor for members of the platelet-derived growth factor family. The PDGFRA gene maps to 4q11-q13 on chromosome 4. The GenBank GeneID for PDGFRA is 5156. FIG. 12 lists some of the PDGFRA amino acid sequences that have been deposited with GenBank under GeneID 5156 as of Jul. 4, 2004. SEQ ID NO:26 corresponds to GenBank accession number BAA08742; SEQ ID NO:27 corresponds to GenBank accession number AAA96715; SEQ ID NO:28 corresponds to GenBank accession number AAA60048; and SEQ ID NO:29 corresponds to GenBank accession number P16234. FIG. 18 shows ClustalW alignments of these PDGFRA amino acid sequences. As used herein a “PDGFRA polypeptide” is a polypeptide that comprises the amino acid sequence of SEQ ID NO:26, 27, 28, 29, a fragment thereof or an allelic variant thereof.

Agonist: As used herein, the term “agonist” refers to an agent that increases or prolongs the duration of the effect of a polypeptide or a nucleic acid. Agonists may include proteins, nucleic acids, carbohydrates, lipids, small molecules, ions, or any other molecules that modulate the effect of the polypeptide or nucleic acid. An agonist may be a direct agonist, in which case it is a molecule that exerts its effect by binding to the polypeptide or nucleic acid, or an indirect agonist, in which case it exerts its effect via a mechanism other than binding to the polypeptide or nucleic acid (e.g., by altering expression or stability of the polypeptide or nucleic acid, by altering the expression or activity of a target of the polypeptide or nucleic acid, by interacting with an intermediate in a pathway involving the polypeptide or nucleic acid, etc.)

Antagonist: As used herein, the term “antagonist” refers to an agent that decreases or reduces the duration of the effect of a polypeptide or a nucleic acid. Antagonists may include proteins, nucleic acids, carbohydrates, or any other molecules that modulate the effect of the polypeptide or nucleic acid. An antagonist may be a direct antagonist, in which case it is a molecule that exerts its effect by binding to the polypeptide or nucleic acid, or an indirect antagonist, in which case it exerts its effect via a mechanism other than binding to the polypeptide or nucleic acid (e.g., by altering expression or stability of the polypeptide or nucleic acid, by altering the expression or activity of a target of the polypeptide or nucleic acid, by interacting with an intermediate in a pathway involving the polypeptide or nucleic acid, etc.)

Allelic variant: As used herein, an allelic variant of a parent gene is a naturally occurring variant of a gene that differs from the parent gene by one or possibly two or more mutations. Mutations may include, but are not limited to, deletions, additions, substitutions, and amplification of regions of genomic DNA that include all or part of a gene. Generally, allelic variants differ by a single mutation. Under certain circumstances mutations within a gene may be silent. The term is also used herein to refer to a polypeptide that is encoded by an allelic variant of a parent gene.

Diagnostic information: As used herein, diagnostic information or information for use in diagnosis is any information that is useful in determining whether a patient has a disease or condition and/or in classifying the disease or condition into a phenotypic category or any category having significance with regards to the prognosis of or likely response to treatment (either treatment in general or any particular treatment) of the disease or condition. Similarly, diagnosis refers to providing any type of diagnostic information, including, but not limited to, whether a subject is likely to have a condition (such as a tumor), information related to the nature or classification of a tumor, information related to prognosis and/or information useful in selecting an appropriate treatment. Selection of treatment may include the choice of a particular chemotherapeutic agent or other treatment modality such as surgery, radiation, etc., a choice about whether to withhold or deliver therapy, etc.

Fragment: For the purposes of the present invention, a fragment of a parent polypeptide is a naturally occurring fragment (e.g., a fragment that is produced by digestion with a digestive protease) or a fragment that is characteristic of the polypeptide (e.g., a peptide that is unique to that polypeptide). Generally, a fragment of the present invention will be missing one or more amino acids from the N- and/or C-terminus of the parent polypeptide. A fragment will typically include 20 or more amino acids, preferably 40 or more amino acids.

Gene: For the purposes of the present invention, the term “gene” has its meaning as understood in the art. However, it will be appreciated by those of ordinary skill in the art that the term “gene” has a variety of meanings in the art, some of which include gene regulatory sequences (e.g., promoters, enhancers, etc.) and/or intron sequences, and others of which are limited to coding sequences. It will further be appreciated that definitions of “gene” include references to nucleic acids that do not encode proteins but rather encode functional RNA molecules such as tRNAs. For the purpose of clarity we note that, as used herein, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid. It will be appreciated that in the context of the present invention a “gene” as defined herein encompasses any nucleotide molecule that encodes a particular polypeptide (i.e., taking into account possible degeneracies in the genetic code).

Gene encoding a DOG1 polypeptide: FIG. 7 lists some of the DOG1 nucleotide sequences that have been deposited with GenBank under GeneID 55107 as of Jul. 4, 2004. SEQ ID NO:1 corresponds to GenBank accession number NM_{—}018043; SEQ ID NO:2 corresponds to GenBank accession number AK001123; SEQ ID NO:3 corresponds to GenBank accession number AK097619; SEQ ID NO:4 corresponds to GenBank accession number AL833582; SEQ ID NO:5 corresponds to GenBank accession number BC027590; and SEQ ID NO:6 corresponds to GenBank accession number BC033036. FIG. 13 shows ClustalW alignments of these DOG1 nucleotide sequences. As used herein, a “gene encoding a DOG1 polypeptide” is a gene that comprises the portion of SEQ ID NO:1, 2, 3, 4, 5 or 6 that encodes a polypeptide. As used herein, a “gene encoding a DOG1 polypeptide” also encompasses genes that comprise an allelic variant of said portion of SEQ ID NO:1, 2, 3, 4, 5 or 6 (including variants that include silent mutations). The terms “gene encoding a DOG1 polypeptide” and “nucleotide molecule encoding a DOG1 polypeptide” are interchangeable.

Gene encoding a KIT polypeptide: FIG. 9 lists some of the KIT nucleotide sequences that have been deposited with GenBank under GeneID 3815 as of Jul. 4, 2004. SEQ ID NO:12 corresponds to GenBank accession number NM_{—}000222; SEQ ID NO:13 corresponds to GenBank accession number BC071593; and SEQ ID NO:14 corresponds to GenBank accession number X06182. FIG. 15 shows ClustalW alignments of these KIT nucleotide sequences. As used herein, a “gene encoding a KIT polypeptide” is a gene that comprises the portion of SEQ ID NO:12, 13 or 14 that encodes a polypeptide. As used herein, a “gene encoding a KIT polypeptide” also encompasses genes that comprise an allelic variant of said portion of SEQ ID NO:12, 13 or 14 (including variants that include silent mutations). The terms “gene encoding a KIT polypeptide” and “nucleotide molecule encoding a KIT polypeptide” are interchangeable.

Gene encoding a PDGFRA polypeptide: FIG. 11 lists some of the PDGFRA nucleotide sequences that have been deposited with GenBank under GeneID 5156 as of Jul. 4, 2004. SEQ ID NO:22 corresponds to GenBank accession number L25829; SEQ ID NO:23 corresponds to GenBank accession number M21574; SEQ ID NO:24 corresponds to GenBank accession number M22734; and SEQ ID NO:25 corresponds to GenBank accession number X76079. FIG. 17 shows ClustalW alignments of these PDGFRA nucleotide sequences. As used herein, a “gene encoding a PDGFRA polypeptide” is a gene that comprises the portion of SEQ ID NO:22, 23, 24 or 25 that encodes a polypeptide. As used herein, a “gene encoding a PDGFRA polypeptide” also encompasses genes that comprise an allelic variant of said portion of SEQ ID NO:22, 23, 24 or 25 (including variants that include silent mutations). The terms “gene encoding a PDGFRA polypeptide” and “nucleotide molecule encoding a PDGFRA polypeptide” are interchangeable.

Gene product or expression product: A gene product or expression product is, in general, an RNA transcribed from the gene or a polypeptide encoded by an RNA transcribed from the gene.

Marker: A marker, as used herein, refers to a gene whose expression is characteristic of a particular tumor subclass. The term may also refer to a product of gene expression, e.g., an RNA transcribed from the gene or a translation product of such an RNA, the production of which is characteristic of a particular tumor subclass. In some cases expression or levels of a marker may be the sole criterion used to define the tumor subclass. In other cases expression or levels of a marker may be combined with other criteria to define the tumor subclass. The statistical significance of the presence or absence of a marker may vary depending upon the particular marker. In some cases the detection of a marker is highly specific in that it reflects a high probability that the tumor is of a particular subclass. This specificity may come at the cost of sensitivity, i.e., a negative result may occur even if the tumor is a tumor that would be expected to express the marker. Conversely, markers with a high degree of sensitivity may be less specific than those with lower sensitivity. Thus it will be appreciated that a useful marker need not distinguish tumors of a particular subclass with 100% accuracy. Furthermore, it will be appreciated that the use of multiple markers may improve the specificity and/or sensitivity with which a tumor can be identified as being of a particular tumor subclass. It is to be understood that a marker for a particular tumor subclass is a gene (or gene product) whose expression is characteristic of a particular tumor subclass, i.e., a gene (or gene product) whose expression is characteristic of some or all of the cells in the tumor.

Positive or negative subclass: As used herein, a tumor belonging to a positive subclass includes cells with a mutated version of a particular marker gene. A tumor belonging to a mutant negative subclass includes cells with a wild-type version of a particular marker gene. Mutant versions of the KIT marker have been identified that have mutations in exon 9, 11, 13 or 17^{11,12}. Mutant versions of the PDGFRA marker have been identified that have mutation in exon 12 and 18¹³. Mutations may result in overexpression or inappropriate expression of the marker gene. Additionally or alternatively mutations may result in an overly activated gene product (e.g., polypeptide). The terms “KIT mutant positive/negative” and “KIT positive/negative” are used interchangeably herein as are the terms “PDGFRA mutant positive/negative” and “PDGFRA positive/negative”.

Prognostic and predictive information: As used herein the terms prognostic and predictive information are used interchangeably to refer to any information that may be used to foretell any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Response: As used herein a response to treatment may refer to any beneficial alteration in a subject's condition that occurs as a result of treatment. Such alteration may include stabilization of the condition (e.g., prevention of deterioration that would have taken place in the absence of the treatment), amelioration of symptoms of the condition, improvement in the prospects for cure of the condition, etc. One may refer to a subject's response or to a tumor's response. In general these concepts are used interchangeably herein. Tumor or subject response may be measured according to a wide variety of criteria, including clinical criteria and objective criteria. Techniques for assessing response include, but are not limited to, clinical examination, chest X-ray, CT scan, MRI, ultrasound, endoscopy, laparoscopy, presence or level of tumor markers in a sample obtained from a subject, cytology, histology. Many of these techniques attempt to determine the size of a tumor or otherwise determine the total tumor burden. Methods and guidelines for assessing response to treatment are discussed in Therasse et al., “New guidelines to evaluate the response to treatment in solid tumors”, European Organization for Research and Treatment of Cancer, National Cancer Institute of the United States, National Cancer Institute of Canada, J. Natl. Cancer Inst., 92(3):205-16, 2000. The exact response criteria can be selected in any appropriate manner, provided that when comparing groups of tumors and/or patients, the groups to be compared are assessed based on the same or comparable criteria for determining response rate. One of ordinary skill in the art will be able to select appropriate criteria.

Sample: As used herein, a sample obtained from a subject may include, but is not limited to, any or all of the following: a cell or cells, a portion of tissue, blood, serum, ascites, urine, saliva, and other body fluids, secretions, or excretions. The term “sample” also includes any material derived by processing such a sample. Derived samples may include nucleotide molecules or polypeptides extracted from the sample or obtained by subjecting the sample to techniques such as amplification or reverse transcription of mRNA, etc.

Specific binding: As used herein, the term refers to an interaction between a target polypeptide (or, more generally, a target molecule) and a binding agent such as an antibody. The interaction is typically dependent upon the presence of a particular structural feature of the target molecule such as an antigenic determinant or epitope recognized by the binding molecule. For example, if an antibody is specific for epitope A, the presence of a polypeptide containing epitope A or the presence of free unlabeled A in a reaction containing both free labeled A and the antibody thereto, will reduce the amount of labeled A that binds to the antibody. It is to be understood that specificity need not be absolute. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target molecule versus the affinity of the binding molecule for other targets, e.g., competitors. If a binding molecule exhibits a high affinity for a target molecule that it is desired to detect and low affinity for non-target molecules, the antibody will likely be an acceptable reagent for immunodiagnostic purposes. Once the specificity of a binding molecule is established in one or more contexts, it may be employed in other, preferably similar, contexts without necessarily re-evaluating its specificity.

Treating a tumor: As used herein, treating a tumor is taken to mean treating a subject who has the tumor.

Tumor subclass: A tumor subclass is the group of tumors that display one or more phenotypic or genotypic characteristics that distinguish members of the group from other tumors.

Tumor sample: The term “tumor sample” as used herein is taken broadly to include cell or tissue samples removed from a tumor, cells (or their progeny) derived from a tumor that may be located elsewhere in the body (e.g., cells in the bloodstream or at a site of metastasis), or any material derived by processing such a sample. Derived tumor samples may include nucleic acids or proteins extracted from the sample or obtained by subjecting the sample to techniques such as amplification or reverse transcription of mRNA, etc.

II. Gastrointestinal Stromal Tumors:

GISTs occur in the wall of the bowel and have been proposed to arise from the interstitial cells of Cajal. The differential diagnosis of these tumors includes desmoid fibromatosis, Schwannoma, leiomyosarcoma, and, in some cases, high grade sarcomas¹. Accurate diagnosis of GISTs is important, because imatinib mesylate (GLEEVEC® manufactured by Novartis, Switzerland) has been shown to significantly inhibit these tumors²⁻⁵.

Currently, the diagnosis of GISTs relies heavily on expression of the KIT marker. Recommendations in the literature emphasize a diffuse, strong KIT immunoreactivity for the diagnosis of GIST⁶, CD34 immunostaining can also aid in the diagnosis, but a subset of cases is immunonegative while many other types of sarcomas are immunoreactive for this marker⁷⁻¹⁰. In the vast majority of GISTs, high levels of KIT expression are accompanied by a KIT mutation in exon 9, 11, 13 or 17^{11,12}. Recently, a subset of GISTs have been found to have PDGFRA mutations rather than KIT mutations^{13,14}. Patients with GISTs containing mutations in PDGFRA belong to the PDGFRA positive subclass and may still benefit from imatinib therapy². However, this subclass of tumors often fail to react with antibodies against KIT and hence may remain undiagnosed as GISTs. Furthermore, identification of PDGFRA positive mutant GISTs currently requires molecular analysis, a laborious process that is not ideal for application in a routine clinical setting. In addition, some GISTs with KIT mutations may have low KIT expression by immunohistochemistry yet will still respond to imatinib therapy¹⁵. There is therefore an urgent need for methods of identifying and classifying these hard to detect subclasses of GISTs.

The inventors recently examined the gene expression profile of GISTs using cDNA microarrays and identified several tumor markers including one that was named “Discovered on GIST 1” (DOG1)¹⁸. DOG1 was found to be highly expressed in KIT positive GISTs. Using immunohistochemistry with antiserum against DOG1 and in situ hybridization with DOG1-specific probes, the inventors have now unexpectedly shown that DOG1 is highly expressed not only in typical KIT positive GISTs but also in PDGFRA positive GISTs and PDGFRA negative, KIT negative (“wild-type”) GISTs. These unexpected results are described in great detail in the Examples and demonstrate the utility of the DOG1 marker for the classification of these hard to detect GISTs.

According to one aspect, the invention therefore provides a method comprising providing a tumor sample; detecting expression or activity of a gene encoding a DOG1 polypeptide in the sample; and classifying the tumor as a gastrointestinal stromal tumor belonging to a PDGFRA positive subclass based on the results of the detecting step. In certain embodiments, the method further comprises detecting expression or activity of a gene encoding a KIT polypeptide in the sample and/or detecting expression or activity of a gene encoding a PDGFRA polypeptide in the sample. According to such embodiments, the classifying step is based on the results of all detecting steps.

In another aspect, the invention provides a method comprising providing a tumor sample; detecting expression or activity of a gene encoding a DOG1 polypeptide in the sample; and classifying the tumor as a gastrointestinal stromal tumor belonging to a KIT negative, PDGFRA negative subclass based on the results of the detecting step. In certain embodiments, the method further comprises detecting expression or activity of a gene encoding a KIT polypeptide in the sample and/or detecting expression or activity of a gene encoding a PDGFRA polypeptide in the sample. According to such embodiments, the classifying step is based on the results of all detecting steps.

In any of the above methods, the tumor sample may be a blood sample, a urine sample, a serum sample, an ascites sample, a saliva sample, a cell, or a portion of tissue. As described in greater detail below, the methods may further comprise providing diagnostic, prognostic, or predictive information based on the classifying step. Classifying may include stratifying the tumor (and thus stratifying a subject having the tumor), e.g., for a clinical trial. In certain embodiments, the methods may further comprise selecting a treatment based on the classifying step. Additionally or alternatively, the methods may further comprise determining the specific mutations within the KIT and/or PDGFRA markers.

III. Detecting Expression or Activity of a Gene Marker:

In one aspect, the invention provides a method of classifying GISTs by detecting the presence of one or more of the inventive gene products encoded by the DOG1, KIT and PDGFRA marker genes. Generally, the inventive classification methods each include a step of detecting expression or activity of a gene encoding a DOG1 polypeptide. As noted, in certain embodiments it may prove advantageous to combine detection of the DOG1 marker with detection of the KIT and/or PDGFRA markers. The gene products are detected in a tumor sample and can be polypeptides or polynucleotides, e.g., mRNA.

Polypeptide Detection:

As is well known in the art, a polypeptide may be detected using any of a variety of techniques and binding agents. Any such technique and agent may be used according to the present invention. In certain preferred embodiments, the binding agent is an antibody that binds specifically to the polypeptide. The invention also encompasses the use of protein arrays, including antibody arrays, for detection of a polypeptide. The use of antibody arrays is described, for example, in Haab et al., “Protein microarrays for highly parallel detection and quantitation of specific proteins and antibodies in complex solutions”, Genome Biol. 2(2):2001, 2001. Other types of protein arrays are known in the art. In general, antibodies that bind specifically to an inventive polypeptide may be generated by methods well known in the art and described, for example, in Harlow, E, Lane, E, and Harlow, E, (eds.) Using Antibodies: A Laboratory Manual, Cold Spring Harbor Laboratory Press, Cold Spring Harbor, 1998. Details and references for the production of antibodies may also be found in U.S. Pat. No. 6,008,337. Antibodies may include, but are not limited to, polyclonal, monoclonal, chimeric (e.g., “humanized”), single chain antibodies, Fab fragments, antibodies generated using phage display technology, etc. The invention encompasses the use of “fully human” antibodies produced using the XENOMOUSE™ technology (AbGenix Corp., Fremont, Calif.) according to the techniques described in U.S. Pat. No. 6,075,181.

In addition, in certain embodiments of the invention the polypeptides are detected using other specific binding agents known in the art for the detection of polypeptides, such as aptamers (Aptamers, Molecular Diagnosis, Vol. 4, No. 4, 1999), reagents derived from combinatorial libraries for specific detection of proteins in complex mixtures, random peptide affinity reagents, etc. In general, any appropriate binding agent for detecting a polypeptide may be used in conjunction with the present invention, although antibodies may represent a particularly appropriate modality.

In certain embodiments of the inventive methods a single binding agent (e.g., antibody) is used whereas in other embodiments of the invention multiple binding agents, directed either against the same or against different polypeptides can be used to increase the sensitivity or specificity of the detection technique or to provide more detailed information than that provided by a single binding agent. Thus the invention encompasses the use of a battery of binding agents that bind to polypeptides encoded by the marker genes identified herein. Of course these agents can also be used in conjunction with binding agents against polypeptides encoded by other useful marker genes (e.g., CD34 when used with GISTs).

In general, the inventive polypeptides are detected within a tumor sample that has been obtained from a subject, e.g., a tissue sample, cell sample, cell extract, body fluid sample, etc. The invention encompasses the recognition that the polypeptides encoded by the marker genes (or portions thereof) may be present in serum, enabling their detection through a blood test rather than requiring a biopsy specimen. Measurement of prostate specific antigen (PSA) in serum using an immunoassay technique is widely used as a method for early detection of prostate cancer and for monitoring recurrence or progression after therapy, etc. One of ordinary skill in the art will readily be able to develop appropriate assays for polypeptides encoded by the marker genes described herein and to apply them to the detection of such polypeptides in serum. Similar methods may be applied to other body fluid samples, e.g., ascites, urine, saliva, etc.

In certain embodiments, binding can be detected by adding a detectable label to the binding agent. In other embodiments, binding can be detected by using a labeled secondary binding agent that associates specifically with the primary binding agent, e.g., as is well known in the art of antigen/antibody detection. The detectable label may be directly detectable or indirectly detectable, e.g., through combined action with one or more additional members of a signal producing system. Examples of directly detectable labels include radioactive, paramagnetic, fluorescent, light scattering, absorptive and colorimetric labels. Indirectly detectable labels include chemiluminescent labels, e.g., enzymes that are capable of converting a substrate to a chromogenic product such as alkaline phosphatase, horseradish peroxidase and the like.

Once a labeled binding agent has bound a polypeptide marker, the complex may be visualized or detected in a variety of ways, with the particular manner of detection being chosen based on the particular detectable label. Representative detection means include, e.g., scintillation counting, autoradiography, measurement of paramagnetism, fluorescence measurement, light absorption measurement, measurement of light scattering and the like. Depending upon the nature of the sample, appropriate detection techniques include, but are not limited to, immunohistochemistry (IHC), radioimmunoassay, ELISA, immunoblotting and fluorescence activated cell sorting (FACS). In the case where the polypeptide is to be detected in a tissue sample, e.g., a biopsy sample, IHC is a particularly appropriate detection technique.

In general, the detection techniques of the present invention will include a negative control, which can involve applying the test to a control sample (e.g., from a normal tissue) so that the signal obtained thereby can be compared with the signal obtained from the tumor sample being tested. In tests in which a secondary binding agent is used to detect a primary binding agent that binds to the polypeptide of interest, an appropriate negative control can involve performing the test on a portion of the sample with the omission of the primary binding agent.

In general, the results of the inventive detection techniques can be presented in any of a variety of formats. The results can be presented in a qualitative fashion. For example, the test report may indicate only whether or not a particular polypeptide marker was detected, perhaps also with an indication of the limits of detection. The results may be presented in a semi-quantitative fashion. For example, various ranges may be defined, and the ranges may be assigned a score (e.g., 0 to 3 as described in the Examples) that provides a certain degree of quantitative information. Such a score may reflect various factors, e.g., the number of cells in which the polypeptide is detected, the intensity of the signal (which may indicate the level of expression of the polypeptide), etc. The results may be presented in a quantitative fashion, e.g., as a percentage of cells in which the polypeptide is detected, as a protein concentration, etc. As will be appreciated by one of ordinary skill in the art, the type of output provided by a test will vary depending upon the technical limitations of the test and the biological significance associated with detection of the polypeptide. For example, in the case of one polypeptide marker a purely qualitative output (e.g., whether or not the polypeptide is detected at a certain detection level) provides significant information. In another case a more quantitative output (e.g., a ratio of the level of expression of the polypeptide in the sample being tested versus the normal level) is necessary.

Polynucleotide Detection:

Although in many cases detection of polypeptides using binding agents such as antibodies represents the most convenient means of determining whether a gene is expressed (or overexpressed) in a particular sample, the invention also encompasses the detection of polynucleotides, e.g., mRNAs for this purpose. Microarray analysis is but one means by which polynucleotides can be used to detect or measure gene expression. Expression of a gene can also be measured by a variety of techniques that make use of a polynucleotide corresponding to part or all of the gene rather than a binding agent for a polypeptide encoded by the gene. Appropriate techniques include, but are not limited to, in situ hybridization, Northern blot, and various nucleic acid amplification techniques such as PCR, quantitative PCR, and the ligase chain reaction. The use of in situ hybridization is described in greater detail in the Examples. PCR and considerations for primer design are well known in the art and are described, for example, in Newton, et al. (eds.) PCR: Essential data Series, John Wiley & Sons; PCR Primer: A Laboratory Manual, Cold Spring Harbor Laboratory Press, Cold Spring Harbor, N.Y., 1995; White, et al. (eds.) PCR Protocols: Current methods and Applications, Methods in Molecular Biology, The Humana Press, Totowa, N.J., 1993.

Detection of Mutations:

The invention also encompasses the detection of mutations within a marker gene or within a regulatory region of a marker gene. In certain embodiments of the invention, detection of mutations can be used to further classify a tumor. Mutations may include, but are not limited to, deletions, additions, substitutions, and amplification of regions of genomic DNA that include all or part of a gene. Methods for detecting such mutations are well known in the art and include direct sequencing, denaturing HPLC and combinations thereof^{13,22}. Mutations may result in overexpression or inappropriate expression of the gene. Additionally or alternatively mutations may result in an overly activated gene product (e.g., polypeptide). Diagnostic KIT mutations have been identified within mutations in exon 9, 11, 13 or 17^{11,12}. Diagnostic PDGFRA mutations have been identified within exon 12 and 18¹³.

IV. Kits

Another aspect of the invention comprises a kit to test for the presence of any of the inventive polypeptides or polynucleotides in a tumor sample. The kit can comprise, for example, an antibody for detection of a polypeptide or a probe for detection of a polynucleotide. In addition, the kit can comprise a reference or control sample, instructions for processing samples, performing the test and interpreting the results, buffers and other reagents necessary for performing the test. In one embodiment the kit comprises one or more antibodies (monoclonal or polyclonal) for DOG1. In some embodiments monoclonal antibodies are preferred. In other preferred embodiments of the invention, the kit comprises a panel of antibodies, e.g., for DOG1 and KIT; for DOG1 and PDGFRA; or for DOG1, KIT and PDGFRA. In certain embodiments of the invention the kit comprises pairs of primers or probes for detecting expression of one or more of the marker genes of the invention, e.g., for DOG1 and KIT; for DOG1 and PDGFRA; or for DOG1, KIT and PDGFRA. In certain embodiments of the invention the kit comprises a cDNA or oligonucleotide array for detecting expression of one or more of the marker genes of the invention.

V. Diagnostics and Methods of Use Thereof

It is well known in the art that different tumors subclasses may be associated with different prognoses. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). For example, differences in the prognosis of patients with KIT positive^{2,6,8,10,12,16} and PDGFRA positive^{13,14,26-34} GISTs have been described. The present invention therefore offers the possibility of providing diagnostic, prognostic, or predictive information based on the classifying methods. The present invention also offers the possibility of analyzing tumor sample archives containing tissue samples that were obtained from patients and stored with information regarding the progress of the patient's disease. In general such archives consist of tumor samples embedded in paraffin blocks. These tumor samples can be analyzed for their expression of polypeptides encoded by the marker genes of the present invention, particularly DOG1. For example, immunohistochemistry can be performed using antibodies that bind to the polypeptides. Tumors belonging to inventive subclasses may then be identified on the basis of this information. It is then possible to correlate the classification of a given tumor with available clinical information, e.g., age at death, length of survival, response to therapy, etc. Once suitable prognostic or predictive correlations are identified, a patient's likely outcome can be predicted based on whether his or her tumor belongs to an inventive subclass.

VI. Therapy Selection and Stratification of Patients for Clinical Trials

Another aspect of the invention relates to the selection of a treatment regimen based on the inventive classification methods. For example, GLEEVEC® is a tyrosine kinase inhibitor that inhibits KIT but also PDGFRA. Although GLEEVEC® seems to produce the best results with KIT mutant positive GISTs, it has been demonstrated that patients with GISTs belonging to the PDGFRA positive subclass may also benefit from treatment with GLEEVEC®². Prior to the present invention such patients were detected using molecular analysis, a laborious process that is not ideal for application in a routine clinical setting. The present invention provides an alternative and more direct method for identifying these patients. Thus, in certain embodiments, the present invention provides a method of classifying a tumor as belonging to a PDGFRA positive subclass and then selecting treatment with GLEEVEC® based on the results of that classification step.

It will be appreciated that the inventive methods may be combined with the selection of other known therapies for this or another inventive GIST subclass (e.g., the KIT negative, PDGFRA negative subclass). In particular, a number of other therapeutics are currently being developed. For example, SU11248 (manufactured by Pfizer, New York, N.Y.) is a small molecule inhibitor of PDGFRA and KIT that is currently in Phase III clinical trials. This drug is predicted to show utility in treating tumors with KIT or PDGFRA mutations. SU11248 also inhibits VEGFR, thereby providing an additional anti-angiogenic effect. RAD001 (manufactured by Novartis, Switzerland) is currently in Phase I clinical trials. RAD001 inhibits mTOR, a downstream target in the AKT pathway. AKT is a survival pathway that is activated by KIT and many other receptors. It is hoped that the simultaneous inhibition of KIT and mTOR using GLEEVEC® and RAD001 will result in increased effectiveness over GLEEVEC® alone. Novartis have also begun Phase I and II clinical trials with PKC412, an inhibitor of protein kinase C (PKC). PKC412 is less specific than GLEEVEC®, inhibiting PKC, and kinases of KIT, VEGF, PDGF⁴². Amgen of Thousand Oaks, Calif. are developing AMG706 that is thought to have a similar mechanism of action as SU11248. Bristol-Myers Squibb of New York, N.Y. are developing BMS-354825 that is an inhibitor of both KIT and PDGFRA.

Another aspect of the invention relates to the use of the inventive classification methods in the identification of therapeutics that are subclass specific. Indeed, it is well known in the art that some tumors respond to certain therapies while others do not. In general there is very little information that may be used to determine, prior to treatment, the likelihood that a specific tumor will respond to a given therapeutic agent. Many compounds have been tested for anti-tumor activity and appear to be effective in only a small percentage of tumors. Due to the current inability to predict which tumors will respond to a given agent, these compounds have not been developed into marketed therapeutics. This problem reflects the fact that current methods of classifying tumors are limited. However, the present invention offers the possibility of identifying tumor subclasses characterized by a significant likelihood of response to a given agent. Tumor sample archives containing tissue samples obtained from patients that have undergone therapy with various agents are available along with information regarding the results of such therapy. As above, these tumor samples can be analyzed for their expression of polypeptides encoded by the marker genes of the present invention. Tumors belonging to different subclasses may then be identified on the basis of this information. It is then possible to correlate the expression of the marker genes with the response of the tumor to therapy, thereby identifying particular compounds that show a superior efficacy in tumors in this subclass as compared with their efficacy in tumors overall or in tumors not falling within that subclass. Once such compounds are identified it will be possible to select patients whose tumors fall into a given subclass for additional clinical trials using these compounds. Such clinical trials, performed on a selected group of patients, are more likely to demonstrate efficacy. The reagents provided herein, therefore, are valuable both for retrospective and prospective trials.

In the case of prospective trials, detection of expression products of one or more of the marker genes may be used to stratify patients prior to their entry into the trial or while they are enrolled in the trial. In clinical research, stratification is the process or result of describing or separating a patient population into more homogeneous subpopulations according to specified criteria. Stratifying patients initially rather than after the trial is frequently preferred, e.g., by regulatory agencies such as the U.S. Food and Drug Administration that may be involved in the approval process for a medication. In some cases stratification may be required by the study design. Various stratification criteria may be employed in conjunction with detection of expression of one or more marker genes. Commonly used criteria include age, family history, lymph node status, tumor size, tumor grade, etc. Other criteria including, but not limited to, tumor aggressiveness, prior therapy received by the patient, etc. Stratification is frequently useful in performing statistical analysis of the results of a trial. Ultimately, once compounds that exhibit superior efficacy against a given GIST subclass are identified, reagents for detecting expression of the inventive marker genes may be used to guide the selection of appropriate chemotherapeutic agent(s).

In summary, by providing reagents and methods for classifying tumors based on their expression of the marker genes, the present invention offers a means to select suitable therapies. It also offers a means of individualizing therapies to specific subclasses of patients. The invention further provides a means to identify a patient population that may benefit from potentially promising therapies that have been abandoned due to inability to identify the patients who would benefit from their use.

VI. Therapeutics

The invention encompasses the use of the inventive marker genes and their expression products as targets for the development of therapeutics. The invention specifically encompasses antagonists of the DOG1 marker gene and its expression products. Such antagonists (which include, but are not limited to, antibodies, small molecules, antisense nucleic acids) may be produced or identified using any of a variety of methods known in the art. For example, a purified polypeptide or fragment thereof may be used to raise antibodies or to screen libraries of compounds to identify those that specifically bind to a DOG1 polypeptide. The fact that DOG1 is a cell membrane associated protein makes it an attractive candidate for antibody therapeutics.

Preferably antibodies suitable for use as therapeutics exhibit high specificity for the target polypeptide and low background binding to other polypeptides. In general, monoclonal antibodies are preferred for therapeutic purposes. Antibodies directed against a polypeptide expressed by a cell may have a number of mechanisms of action. In certain instances, e.g., in the case of a polypeptide that exerts a growth stimulatory effect on a cell, antibodies may directly antagonize the effect of the polypeptide and thereby arrest tumor progression, trigger apoptosis, etc. While not wishing to be bound by any theory, it may be that DOG1 has a growth stimulatory effect on tumor cells or facilitates the growth of such cells in some other way, e.g., by enhancing angiogenesis, by allowing cells to overcome normal growth regulatory mechanisms, or by blocking mechanisms that would normally lead to elimination of mutated or otherwise abnormal cells.

In certain embodiments of the invention the antibody may serve to target a toxic moiety to the cell. Thus the invention encompasses the use of antibodies that have been conjugated with a cytotoxic agent, e.g., a toxin such as ricin or diphtheria toxin, a radioactive moiety, etc. Such antibodies can be used to direct the cytotoxic agent specifically to cells that express a DOG1 polypeptide.

Although certain antagonists may function through direct interaction with a polypeptide, e.g., by inhibiting its activity, others may function by affecting expression of the polypeptide. Reduction in expression of an endogenously produced polypeptide may be achieved by the administration of antisense nucleic acids (e.g., oligonucleotides, RNA, DNA, most typically oligonucleotides that have been modified to improve stability or targeting) or peptide nucleic acids comprising sequences complementary to those of the mRNA that encodes the polypeptide. Antisense technology and its applications are described in Phillips, M I (ed.) Antisense Technology, Methods Enzymol., Volumes 313 and 314, Academic Press, San Diego, 2000, and references mentioned therein. Ribozymes (catalytic RNA molecules that are capable of cleaving other RNA molecules) represent another approach to reducing gene expression. Such ribozymes can be designed to cleave specific mRNAs corresponding to a gene of interest. Their use is described in U.S. Pat. No. 5,972,621, and references therein. The invention encompasses the delivery of antisense and/or ribozyme molecules via a gene therapy approach in which vectors or cells expressing the antisense molecules are administered to an individual.

Small molecule modulators (e.g., inhibitors or activators) of gene expression are also within the scope of the invention and may be detected by screening libraries of compounds using, for example, cell lines that express a DOG1 polypeptide or a version of a DOG1 polypeptide that has been modified to include a readily detectable moiety. Methods for identifying compounds capable of modulating gene expression are described, for example, in U.S. Pat. No. 5,976,793.

More generally, the invention encompasses compounds that modulate the activity of a marker gene of the present invention. Methods of screening for such interacting compounds are well known in the art and depend, to a certain degree, on the particular properties and activities of the polypeptide encoded by the gene. Representative examples of such screening methods may be found, for example, in U.S. Pat. No. 5,985,829, U.S. Pat. No. 5,726,025, U.S. Pat. No. 5,972,621, and U.S. Pat. No. 6,015,692. The skilled practitioner will readily be able to modify and adapt these methods as appropriate for a given polypeptide. Thus the invention encompasses methods of screening for molecules that modulate the activity of a polypeptide encoded by a marker gene, particularly the DOG1 gene.

The invention also encompasses the use of polynucleotide sequences corresponding to marker genes, or portions thereof, as DNA vaccines. Such vaccines comprise polynucleotide sequences, typically inserted into vectors, that direct the expression of an antigenic polypeptide within the body of the individual being immunized. Details regarding the development of vaccines, including DNA vaccines for various forms of cancer may be found, for example, in Brinckerhoff L H, Thompson L W, Slingluff C L, Melanoma Vaccines, Curr. Opin. Oncol., 12(2):163-73, 2000 and in Stevenson F K, DNA vaccines against cancer: from genes to therapy, Ann. Oncol., 10(12): 1413-8, 1999 and references cited therein. The polypeptides, or fragments thereof, that are encoded by marker genes may also find use as cancer vaccines. Such vaccines may be used for the prevention and/or treatment of cancer.

The invention includes pharmaceutical compositions comprising the inventive antibodies, or small molecule inhibitors, agonists, or antagonists described above. In general, a pharmaceutical composition will include an active agent in addition to one or more inactive agents such as a sterile, biocompatible carrier including, but not limited to, sterile water, saline, buffered saline, or dextrose solution. The pharmaceutical compositions may be administered either alone or in combination with other therapeutic agents including other chemotherapeutic agents, hormones, vaccines, and/or radiation therapy. By “in combination with”, it is not intended to imply that the agents must be administered at the same time or formulated for delivery together, although these methods of delivery are within the scope of the invention. In general, each agent will be administered at a dose and on a time schedule determined for that agent. Additionally, the invention encompasses the delivery of the inventive pharmaceutical compositions in combination with agents that may improve their bioavailability, reduce or modify their metabolism, inhibit their excretion, or modify their distribution within the body. Alternatively or additionally, inventive pharmaceutical compositions may be administered together with one or more other agents that address a symptom or cause of the disease or disorder being treated, or of any other ailment from which the patient suffers. The invention encompasses treating cancer, particularly breast cancer, by administering the pharmaceutical compositions of the invention. Although the pharmaceutical compositions of the present invention can be used for treatment of any subject (e.g., any animal) in need thereof, they are most preferably used in the treatment of humans.

The pharmaceutical compositions of this invention can be administered to humans and other animals by a variety of routes including oral, intravenous, intramuscular, intraarterial, subcutaneous, intraventricular, transdermal, rectal, intravaginal, intraperitoneal, topical (as by powders, ointments, or drops), bucal, or as an oral or nasal spray or aerosol. In general the most appropriate route of administration will depend upon a variety of factors including the nature of the compound (e.g., its stability in the environment of the gastrointestinal tract), the condition of the patient (e.g., whether the patient is able to tolerate oral administration), etc. At present the intravenous route is most commonly used to deliver therapeutic antibodies and nucleic acids. However, the invention encompasses the delivery of the inventive pharmaceutical composition by any appropriate route taking into consideration likely advances in the sciences of drug delivery.

### EXAMPLES

Materials and Methods:

Tissue Microarrays

The studies described here were performed with the approval of the Institutional Review Board at Stanford University Hospital. Two tissue microarrays (TMAs) were used for this study. The first TMA contained 460 different soft tissue tumors from 421 patients, with each tumor represented by two cores. The samples were distributed over two array blocks that were constructed using a technique previously described¹⁹ with a tissue arrayer from Beecher Instruments, Silver Spring, Md. 0.6 mm cores were taken from paraffin embedded soft tissue tumors archived from the Stanford University Medical Center between 1995 and 2001. This array has also been used for characterization of Apolipoprotein D expression 20. The second TMA used GISTs that were obtained from the pathology archives of Oregon Health and Science University Hospital, the Portland Va. Medical Center and the Kaiser Permanente Northwest Regional Laboratory. This single-block array consisted of 0.6 mm cores from formalin-fixed, paraffin-embedded tumors assembled using a semi-automated tissue arrayer²¹. There was one core for each tumor, and all of the GISTs on this TMA were analyzed for mutations in exons 9, 11, 13 and 17 of the KIT gene using a combination of denaturing HPLC and direct sequencing, as previously described^{13,22}. KIT wild-type tumors included on the array were also screened for mutations in exons 12 and 18 of the PDGFRA gene¹³.

Antibody Generation

The cDNA-derived polypeptide sequence of DOG1 (see FIG. 7) showed no significant homology with polypeptide sequences derived from other genes, including the KIT gene (see FIG. 9). A rabbit polyclonal antibody was raised by injecting three peptides that were selected from within the polypeptide sequence using a program that uses the Hopp/Woods method (described in Hopp and Woods, Mol. Immunol. 20:483, 1983 and Hopp and Woods, Proc. Nat. Acad. Sci. U.S.A. 78:3824, 1981). The peptides were synthesized by standard FMOC chemistry:
Peptide 1-EEAVKDHPRAEYEARVLEKSLK (SEQ ID NO:30);
Peptide 2-DHEECVKRKQRYEVDYNLE (SEQ ID NO:31); and
Peptide 3-KEKVLMVELFMREEQDK (SEQ ID NO:32).

These peptides have no sequence homology to KIT. The peptides were conjugated to keyhole limpet hemocyanin (KLH) and injected into two out-bred rabbits. The serum (S284) was harvested after the rabbits demonstrated a significant anti-peptide titer. Affinity-purified antibodies were obtained by passing the antiserum over an affinity column conjugated with the three peptides; bound antibodies were eluted with a pH gradient.

Immunohistochemistry

Primary antibodies were directed towards DOG1(S284, Applied Genomics, Inc., Huntsville, Ala., Rabbit polyclonal, 1:50) and KIT (DAKO, Carpinteria, Calif., Rabbit polyclonal, 1:50). Serial sections of 4 μm were cut from the tissue array blocks, deparaffinized in xylene, and hydrated in a graded series of alcohol. Staining was then performed using the EnVision+anti-rabbit system (DAKO).

In Situ Hybridization

In situ hybridization of TMA sections was performed based on a protocol published previously^{23,24}. Briefly, digoxigenin (DIG)-labeled sense and anti-sense RNA probes were generated by PCR amplification of 400 to 600 bp products with the T7 promoter incorporated into the primers. In vitro transcription was performed with a DIG RNA-labeling kit and T7 polymerase according to the manufacturer's protocol (Roche Diagnostics, Indianapolis, Ind.). 5 μm thick sections cut from the paraffin blocks, deparaffinized in xylene, were hydrated in graded concentrations of ethanol for 5 minutes each. Sections were then incubated with 1% hydrogen peroxide, followed by digestion in 10 μg/ml of proteinase K at 37° C. for 30 minutes. Sections were hybridized overnight at 55° C. with either sense or antisense riboprobes at 200 ng/ml dilution in mRNA hybridization buffer (DAKO). The following day, sections were washed in 2×SSC and incubated with 1:35 dilution of RNase A cocktail (Ambion, Austin, Tex.) in 2×SSC for 30 minutes at 37° C. Next, sections were stringently washed in 2×SSC/50% formamide twice, followed by one wash at 0.08×SSC at 50° C. Biotin blocking reagents (DAKO) were applied to the section to block the endogenous biotin. For signal amplification, a HRP-conjugated rabbit anti-DIG antibody (DAKO) was used to catalyze the deposition of biotinyl tyramide, followed by secondary streptavidin complex (GenPoint kit; DAKO). The final signal was developed with DAB (GenPoint kit; DAKO), and the tissues were counterstained in hematoxylin for 15 seconds.

Scoring of Immunohistochemistry and In Situ Hybridization

Cores were scored as follows. A score of “0” was given for absent or insignificant staining: less than 5% tumor cells with light brown staining. A score of “1” was given for unscorable cores. A score of “2” was given for light brown stain in greater than 5% of tumor cells or dark brown stain in less than 50% of tumor cells. A score of “3” was given for dark brown staining in greater that 50% tumor cells. Non-tumor cells and cells of unknown origin were not scored. The cores were independently reviewed by two pathologists (Robert B. West and Matt van de Rijn) and disagreements were reviewed together to achieve a consensus score.

Digital Image Collection and Data Analysis

To aid in the analysis of numerous tissue cores stained by immunohistochemistry and in situ hybridization, digital images were collected using the BLISS instrument (Bacuslabs, Lombard Ill.). Scoring results were combined using Deconvoluter and represented in Treeview²⁵, as shown on the website: microarray-pubs.stanford.edu/tma_portal/dog1 where over 4,000 digital images are available.

Results:

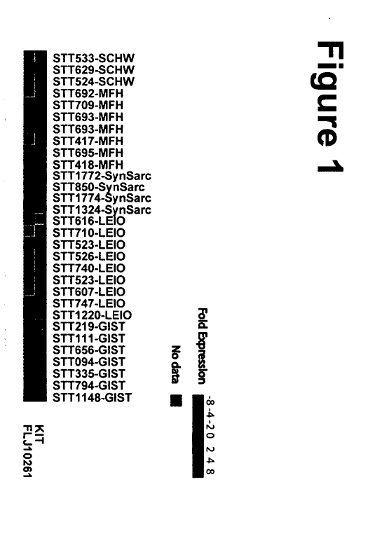

As noted above, the inventors have examined the gene expression profile of GISTs using cDNA microarrays and identified a number of the genes, in addition to the KIT gene, that demonstrated a specific pattern of elevated mRNA expression in GISTs 18. FIG. 1 shows the relative level of mRNA expression for one of these genes, DOG1 (also referred to as FLJ10261), compared with KIT in a variety of soft tissue tumors, including those in the differential diagnosis of GIST. Searches failed to show any sequence similarity between the genes on either the DNA or protein level.

As described above, rabbit antiserum was generated against synthetic peptides derived from the putative coding sequence of DOG1. Antiserum immunoreactivity was characterized on two separate TMAs containing soft tissue tumors. The first TMA contained 460 different soft tissue tumor samples representing over 50 different diagnostic entities²⁰. This array included 22 KIT-immunoreactive GISTs. The second TMA included 127 GIST cases for which the KIT and PDGFRA mutation status had been previously determined. On this TMA there were 102 cases with an activating mutation in KIT, 8 cases with a mutation in PDGFRA, and 17 cases that were wild-type for both kinases but nevertheless had clinical, histologic, and immunophenotypic features typical for GIST.

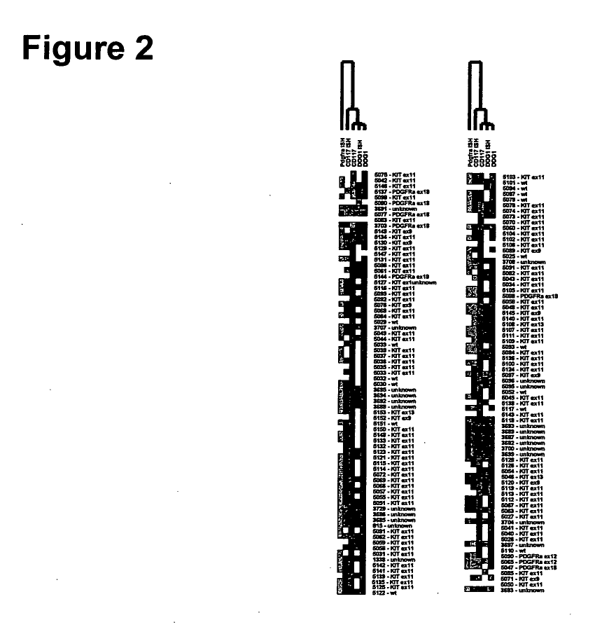

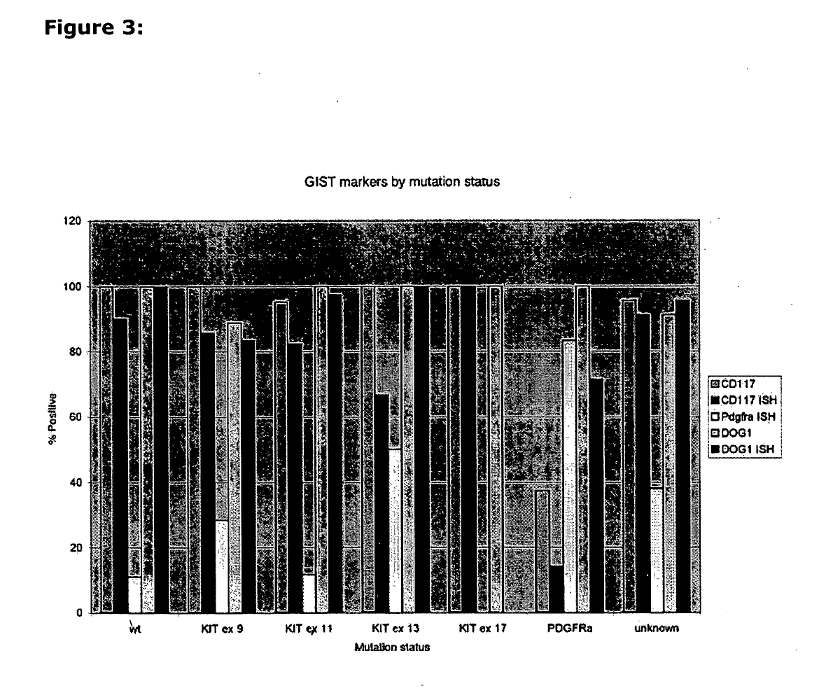

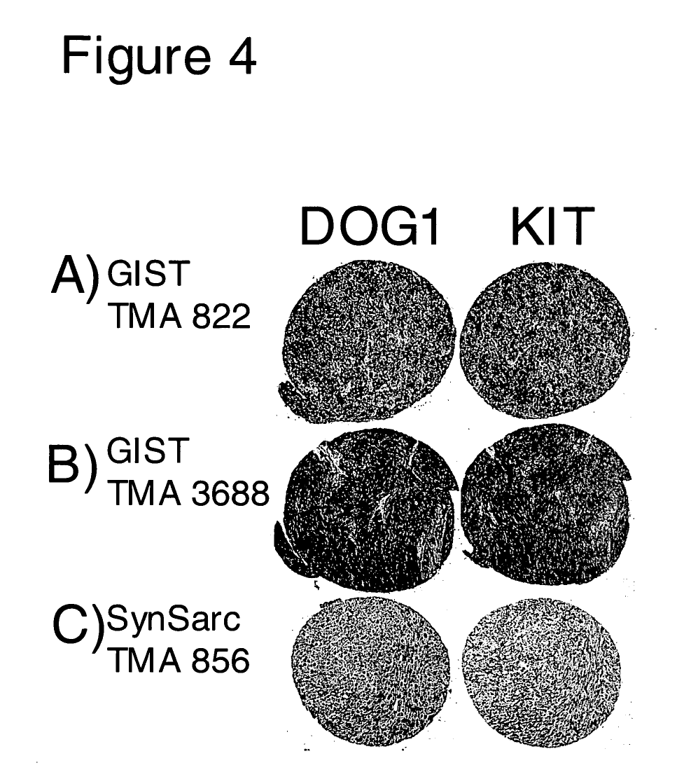

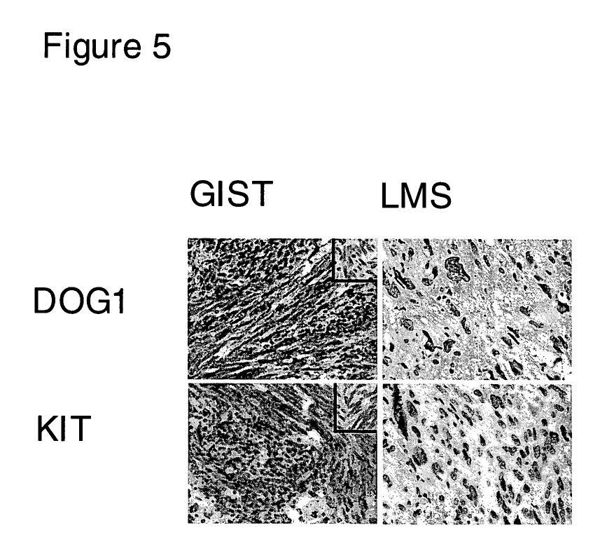

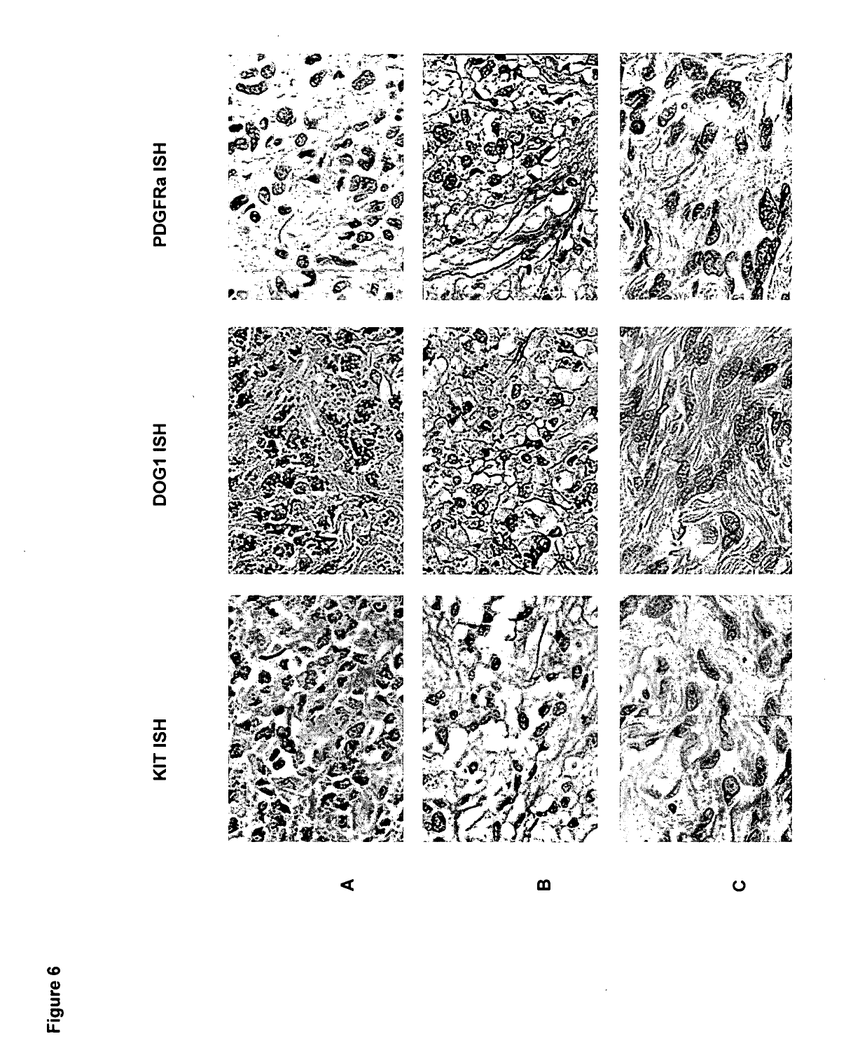

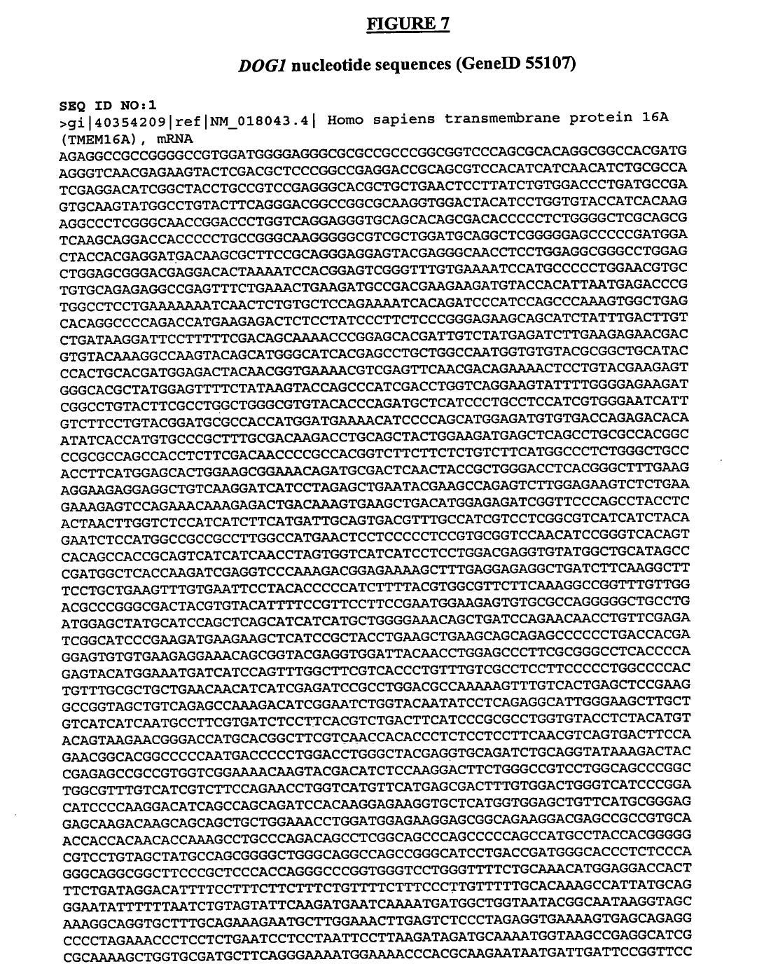

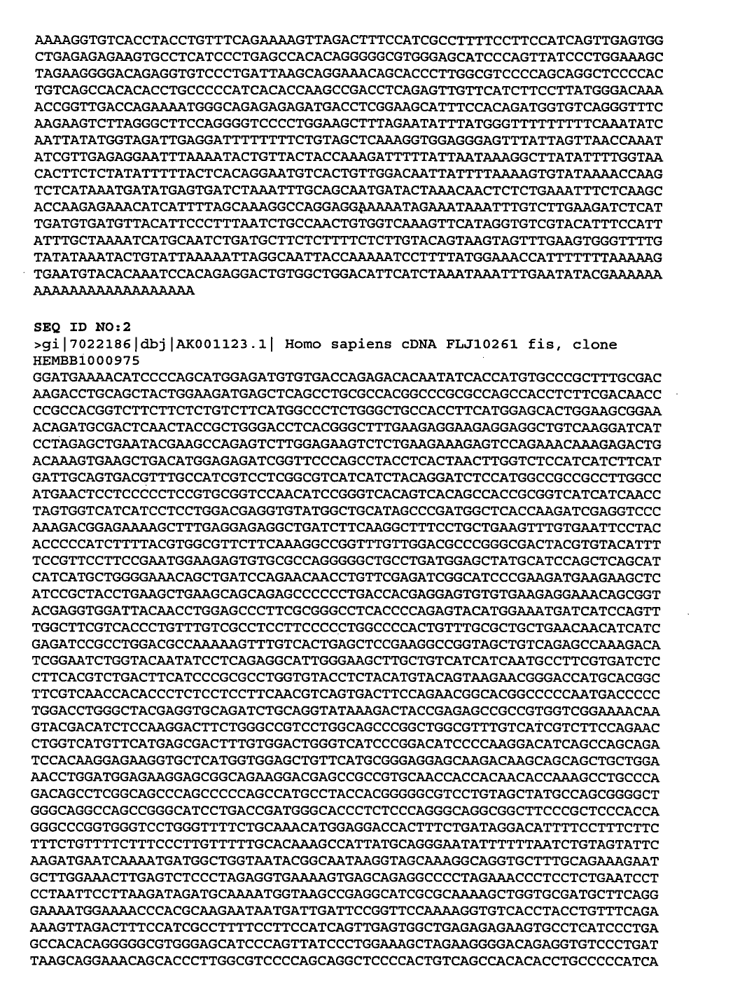

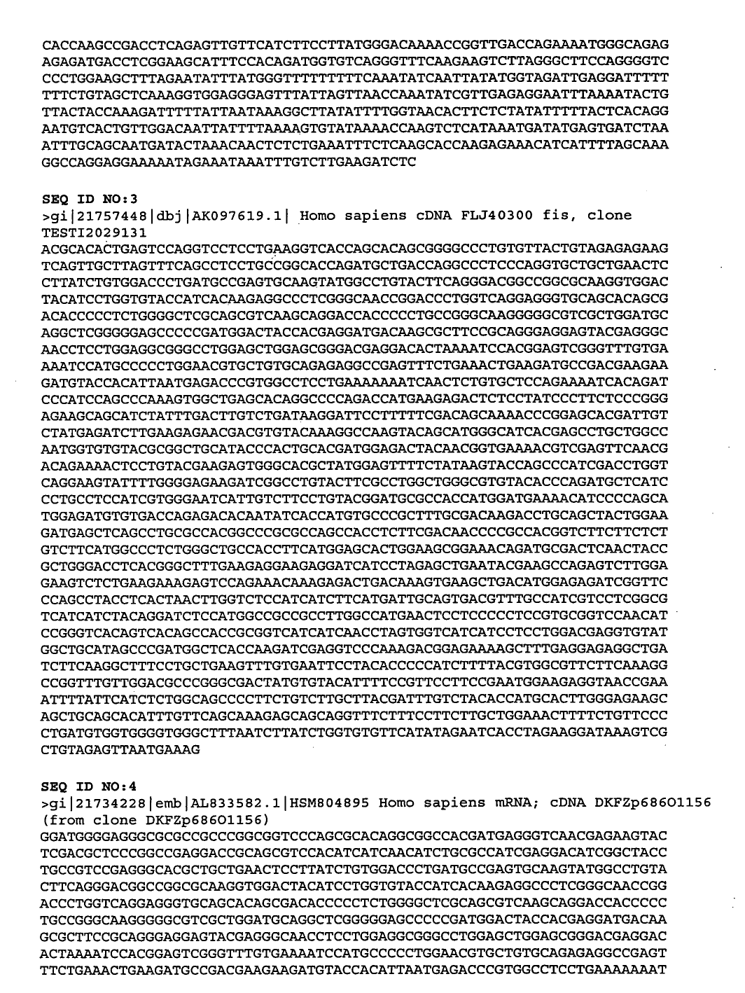

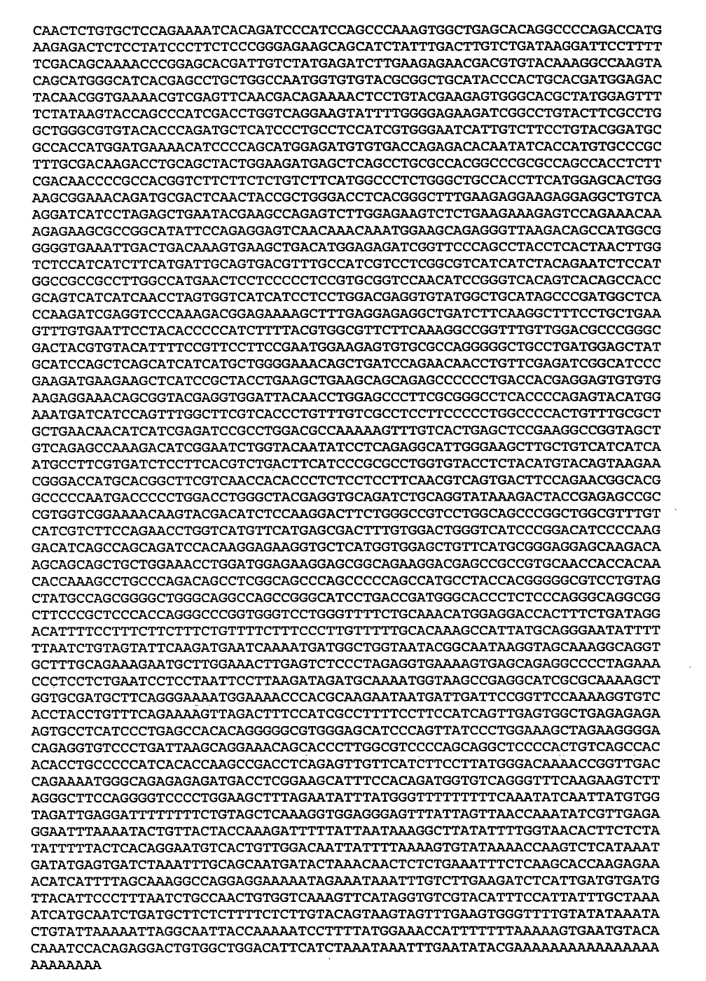

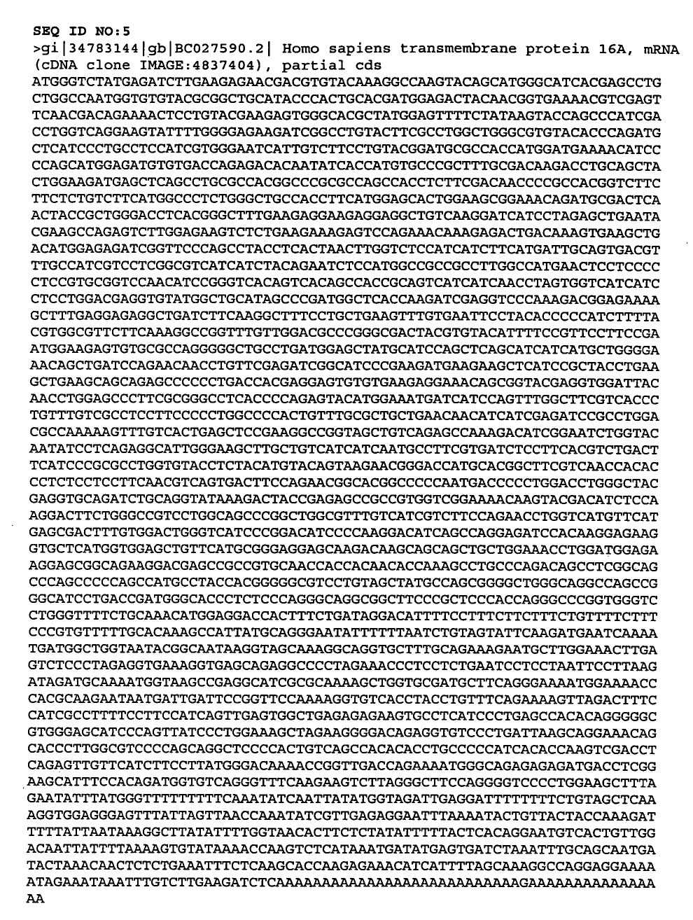

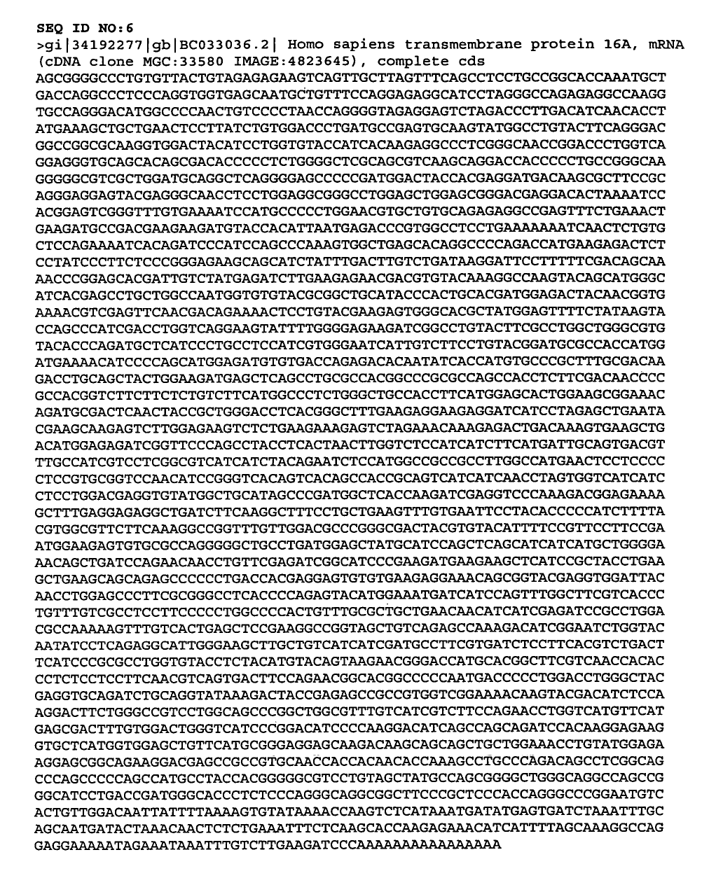

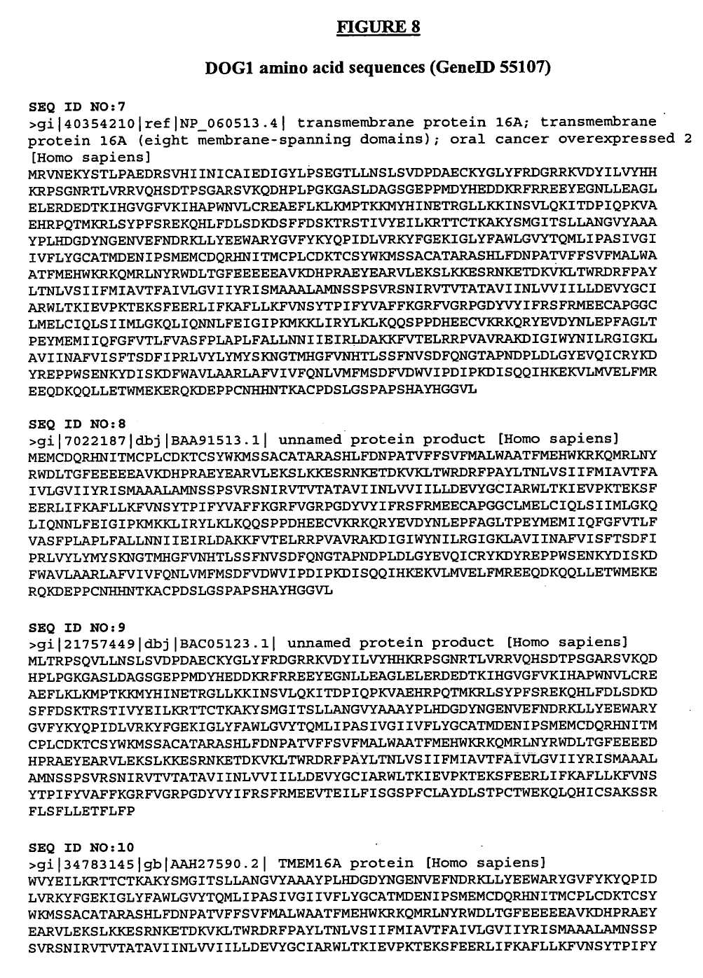

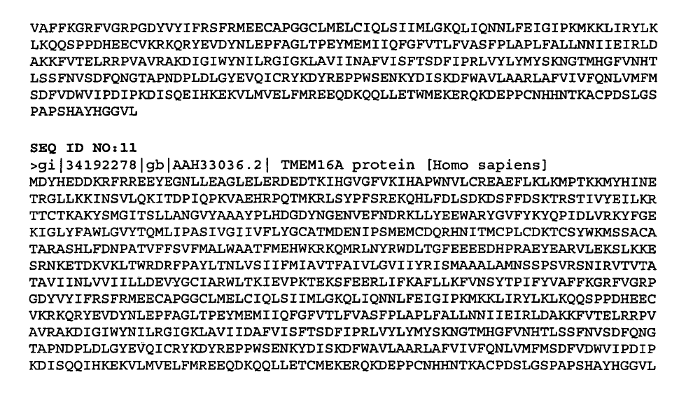

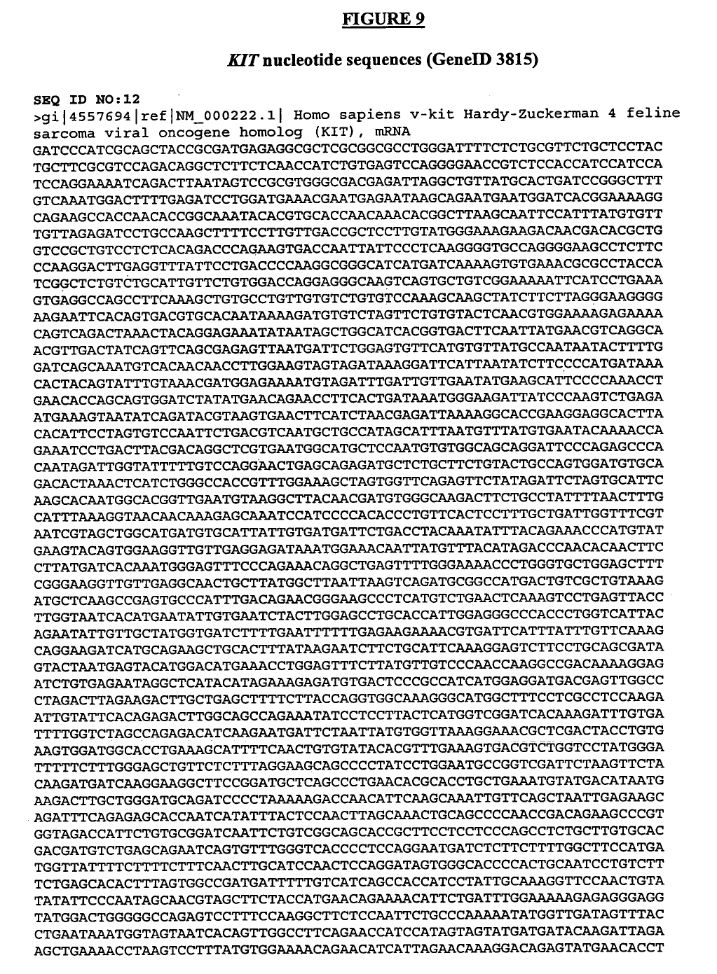

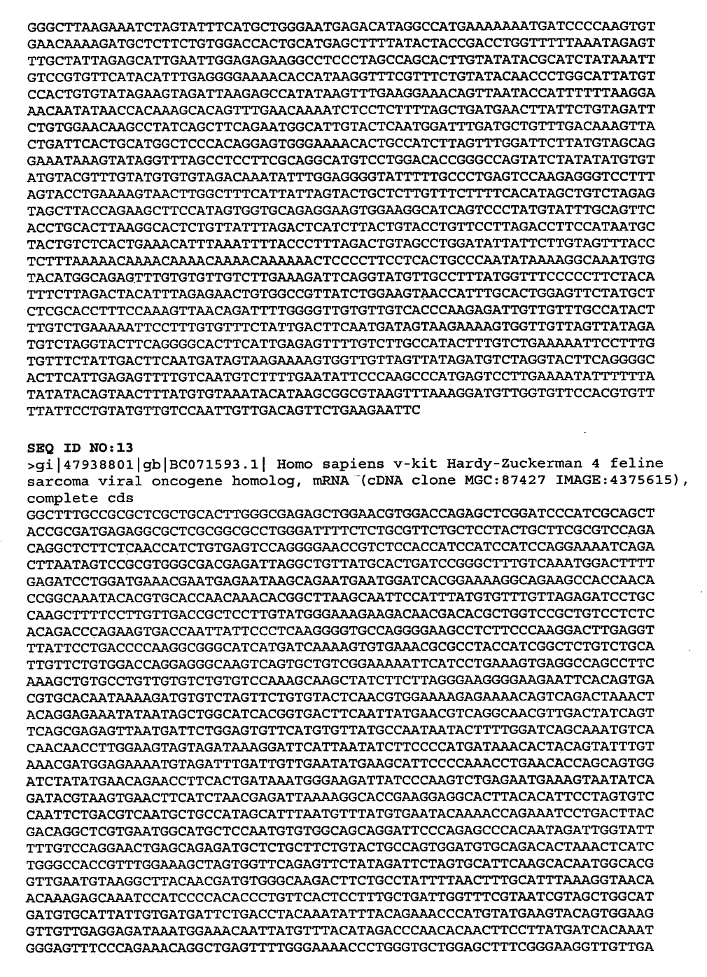

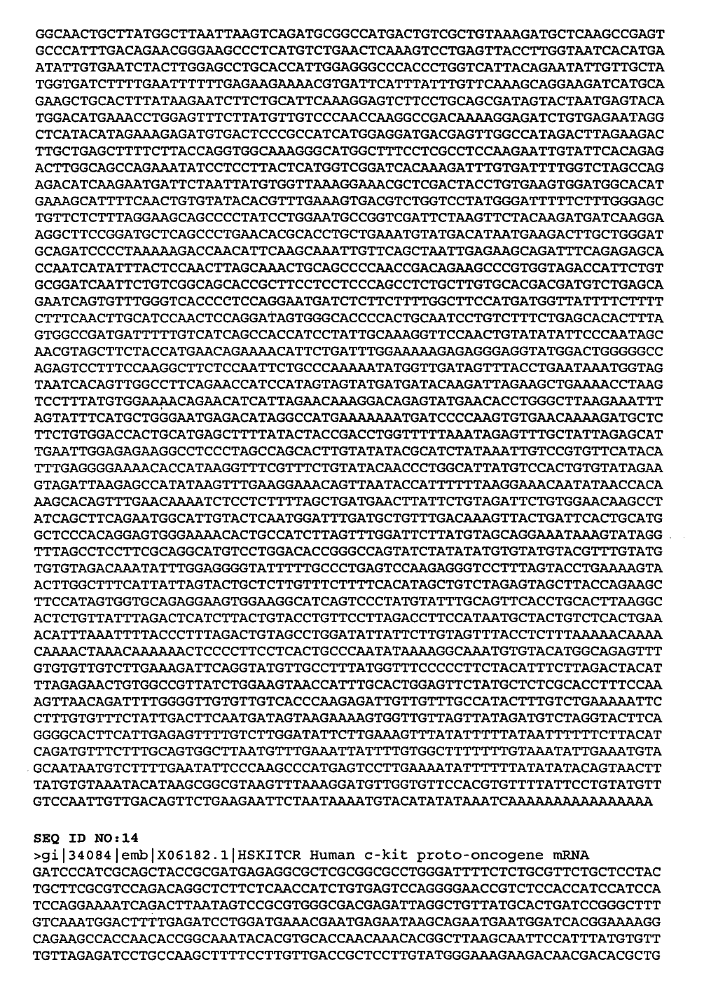

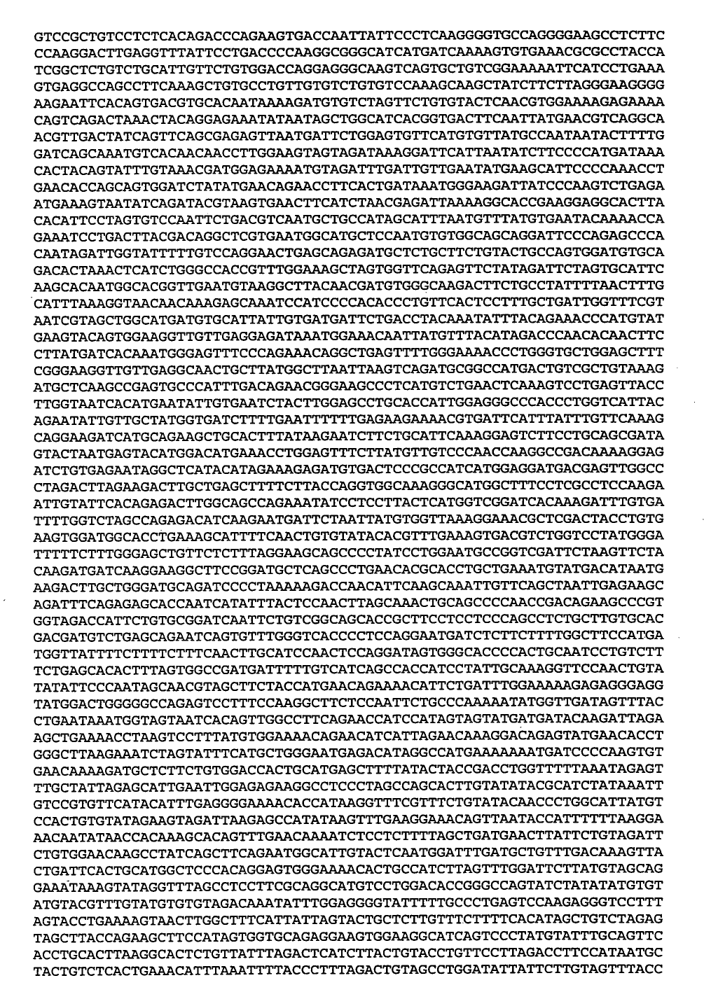

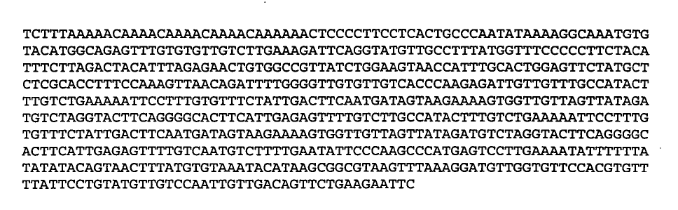

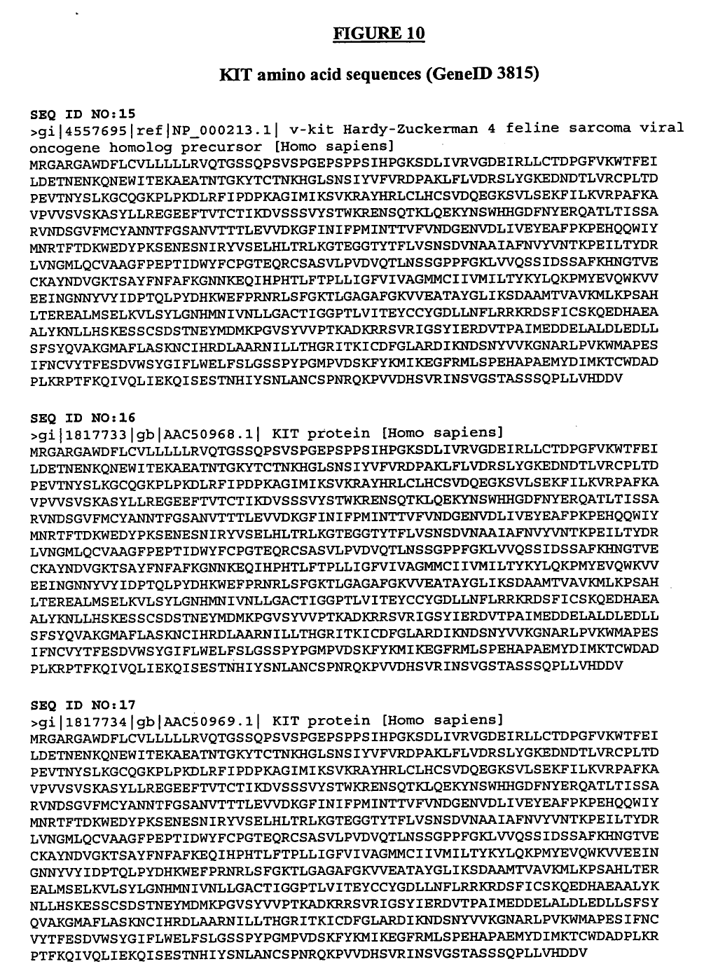

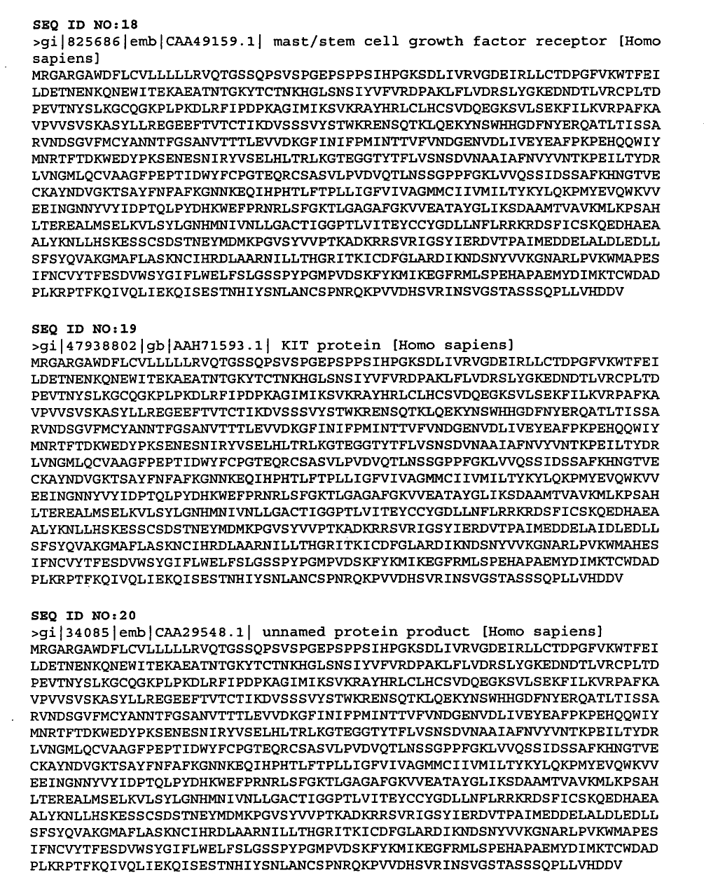

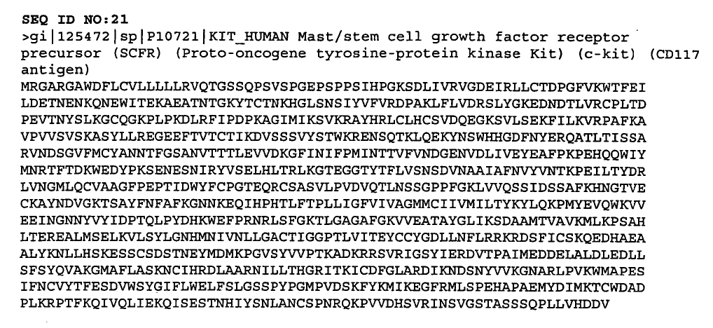

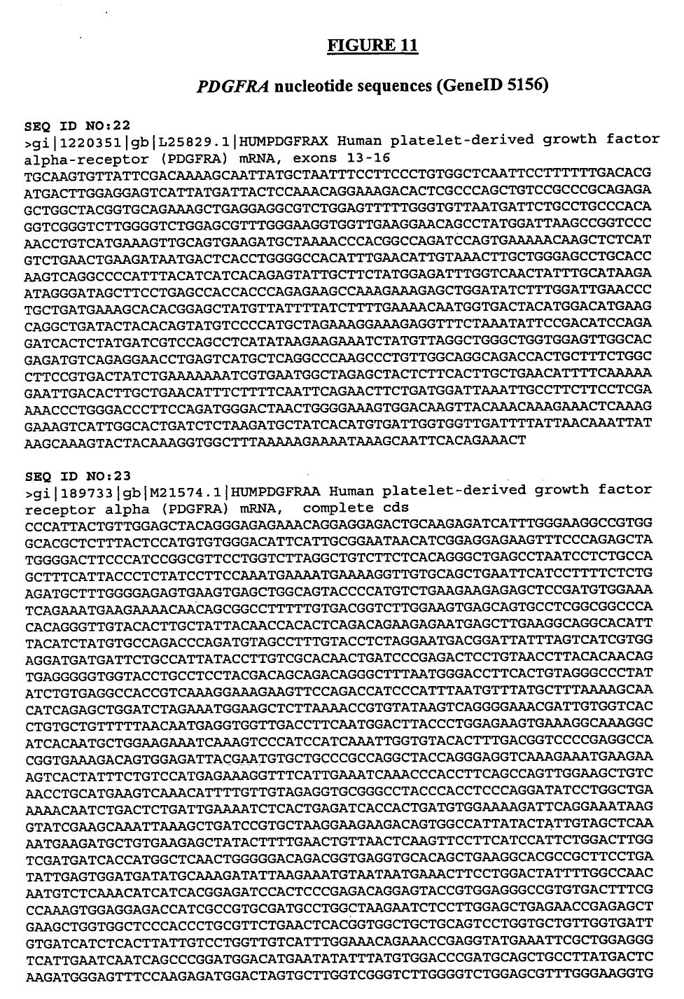

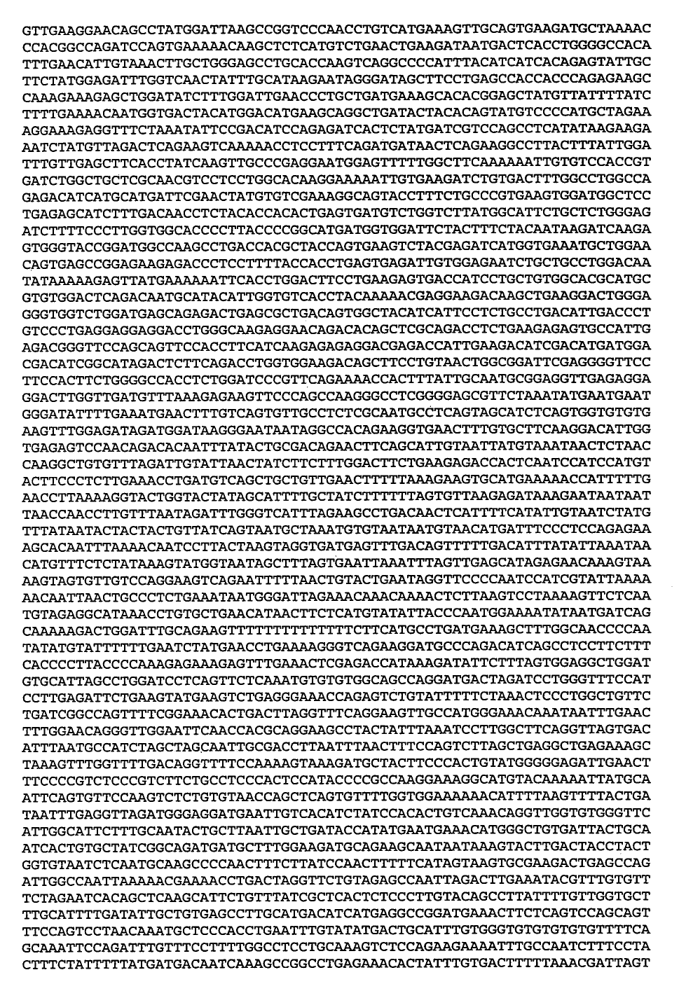

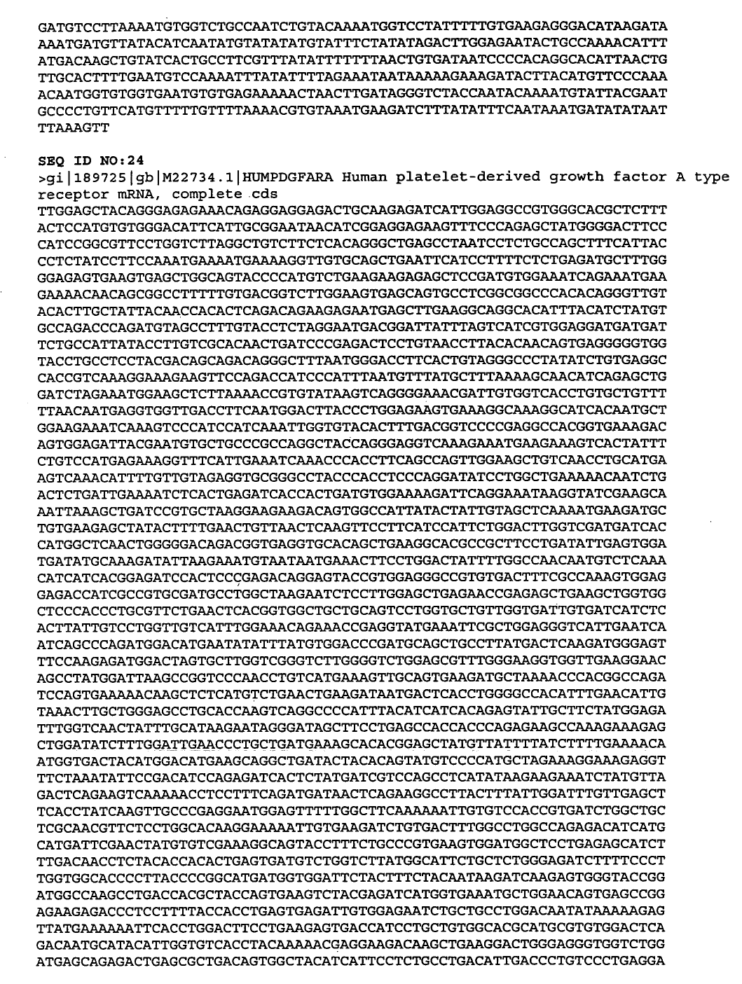

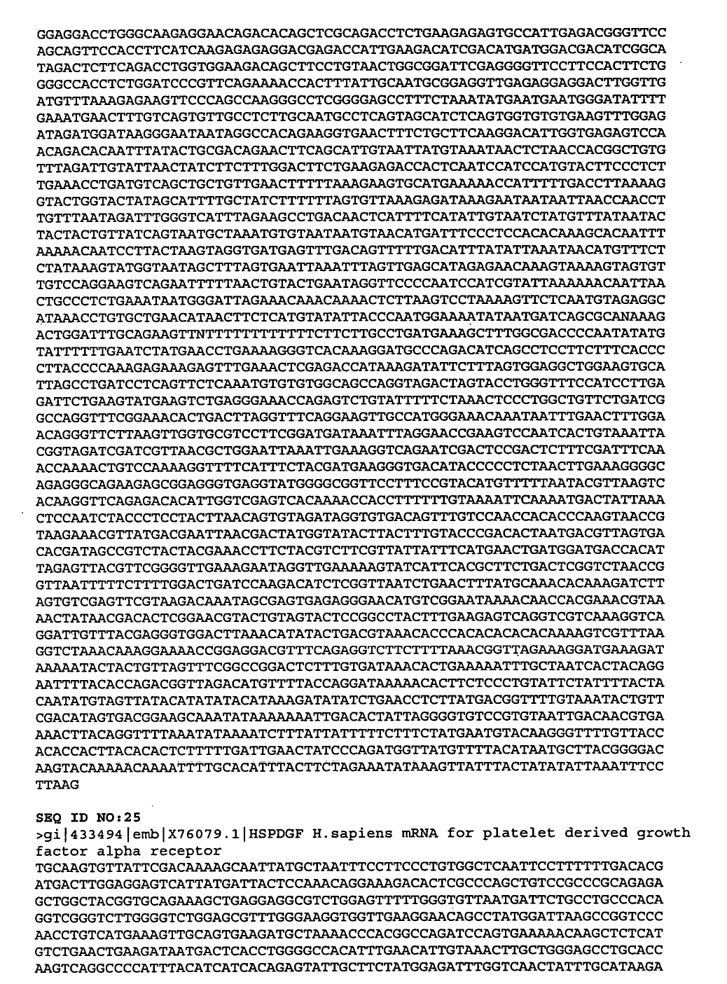

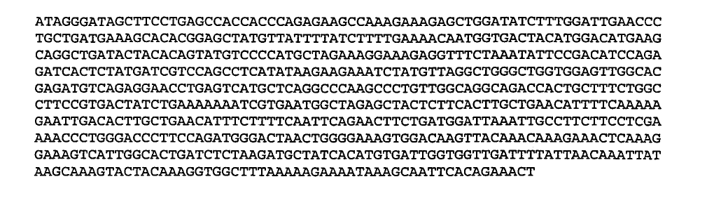

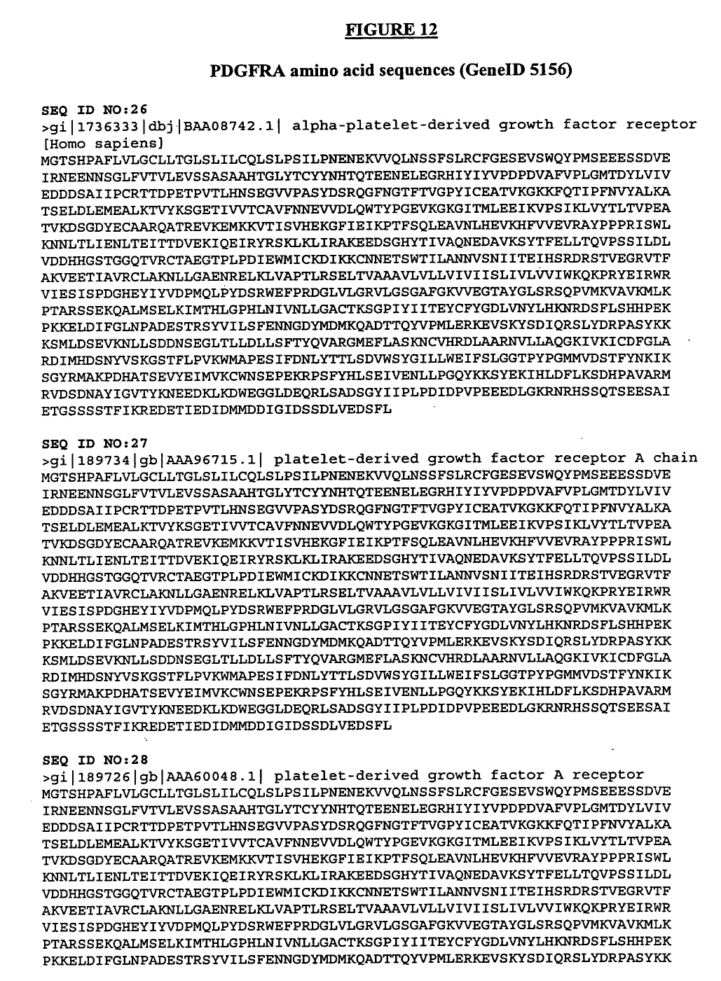

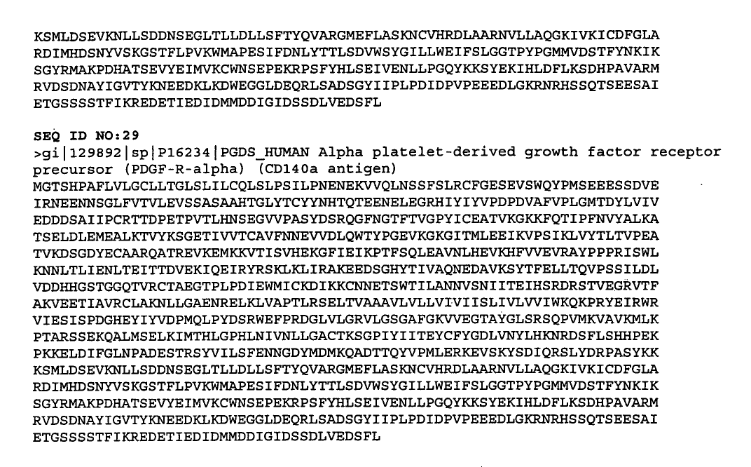

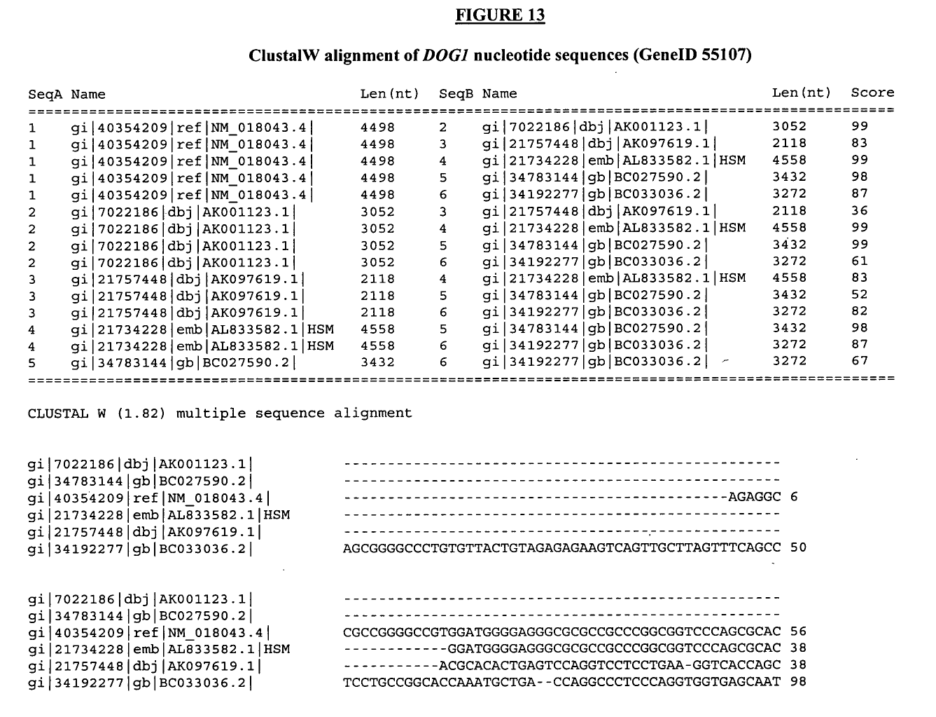

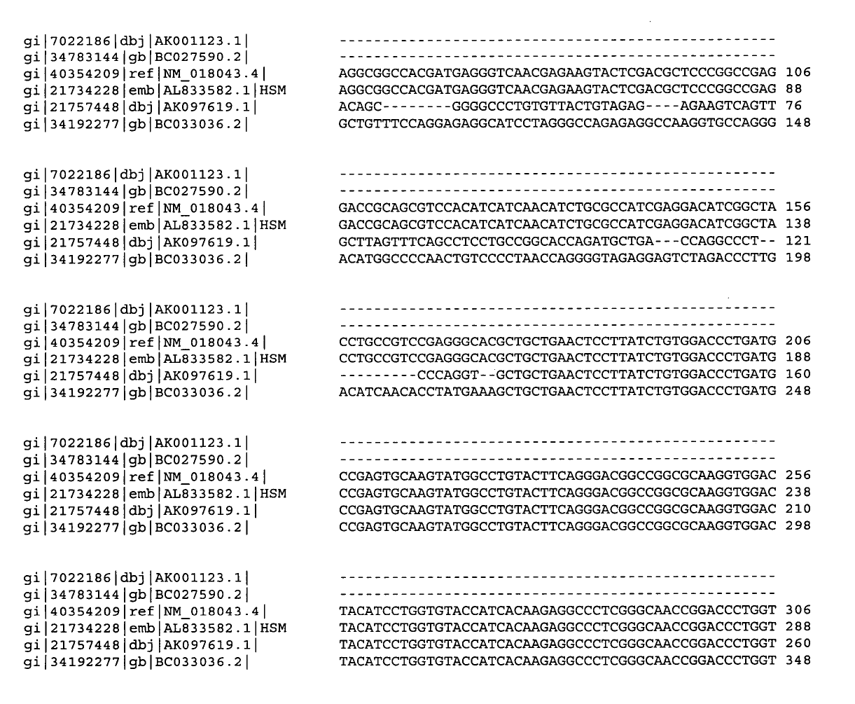

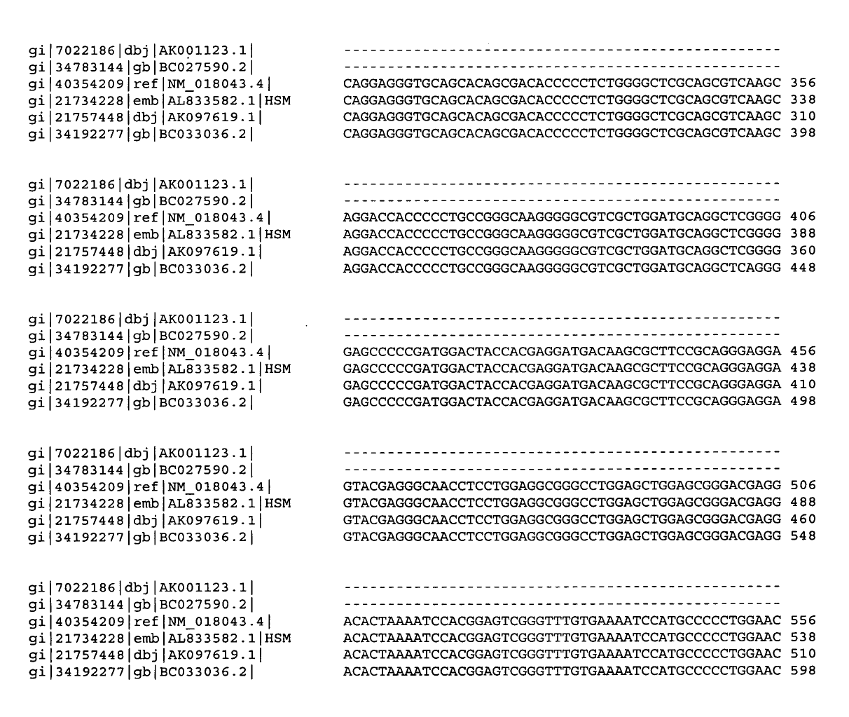

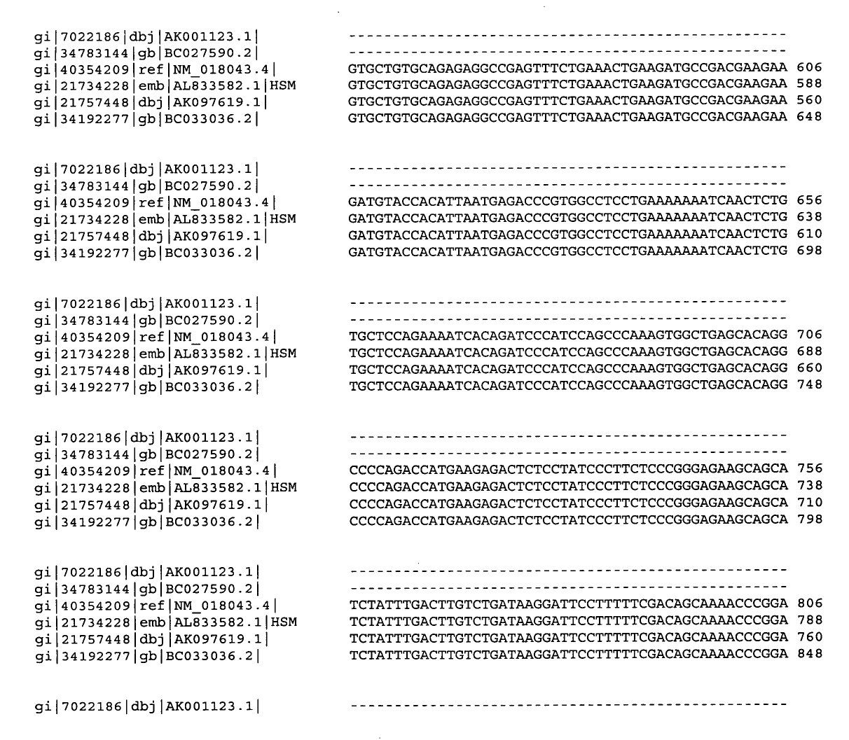

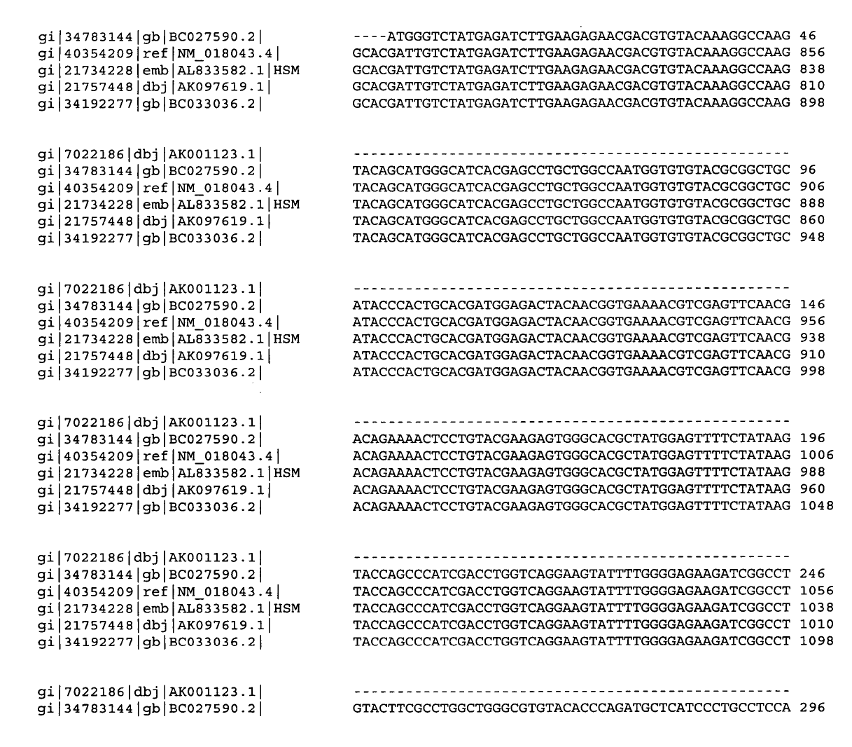

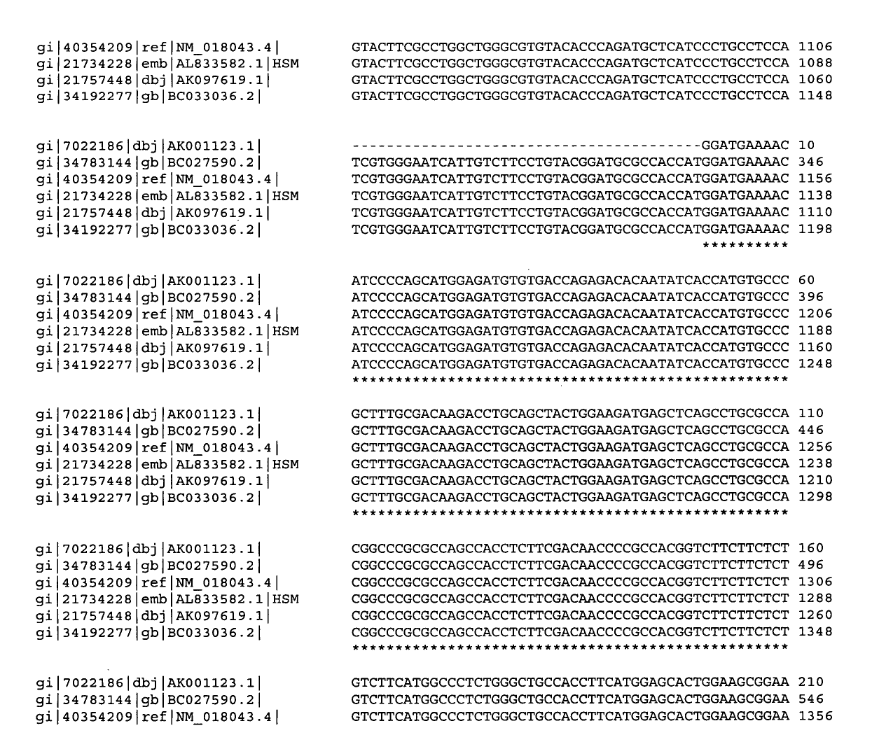

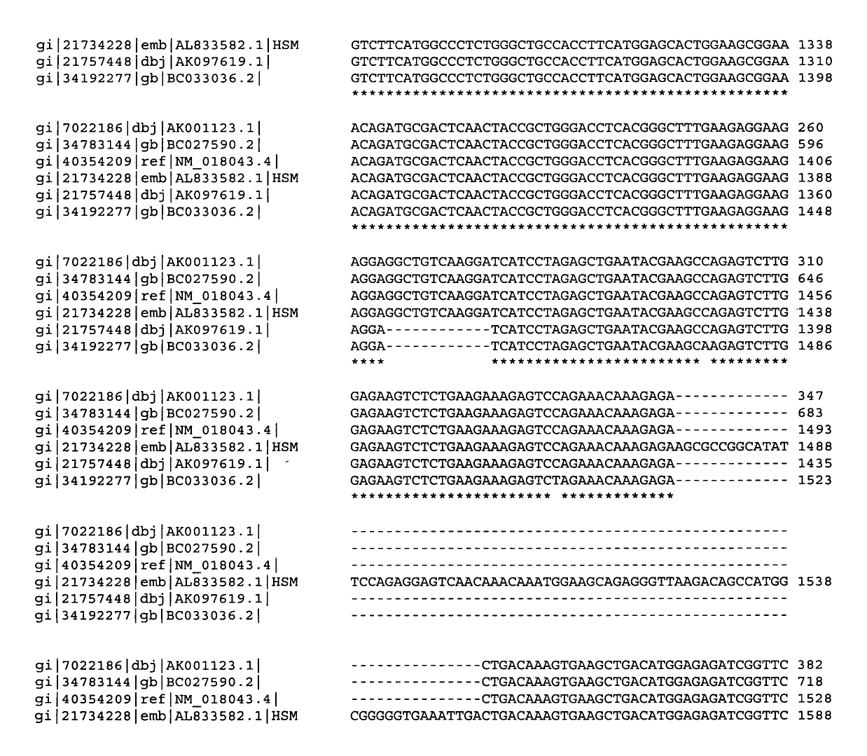

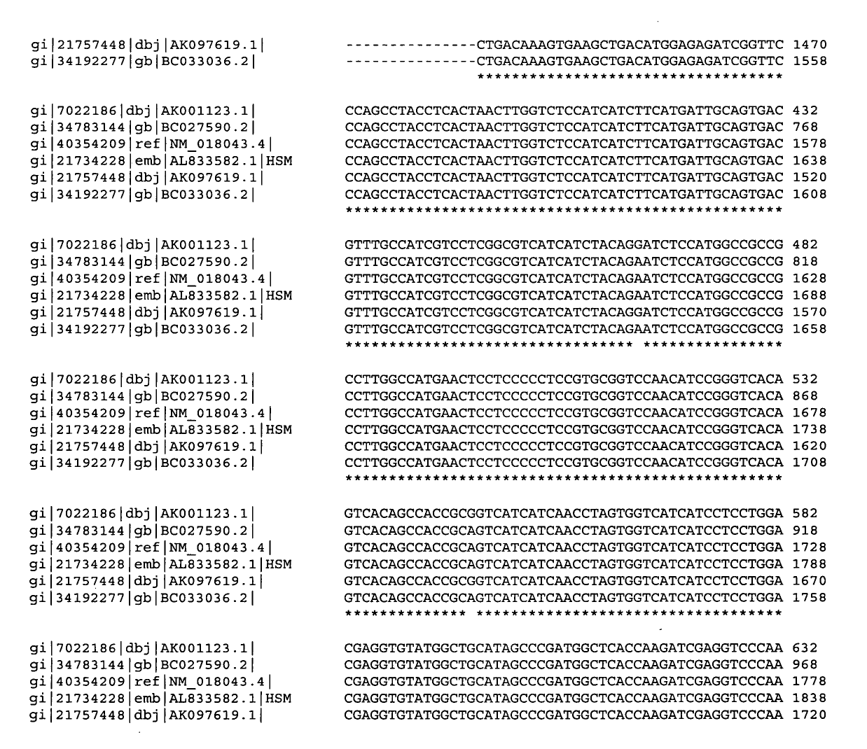

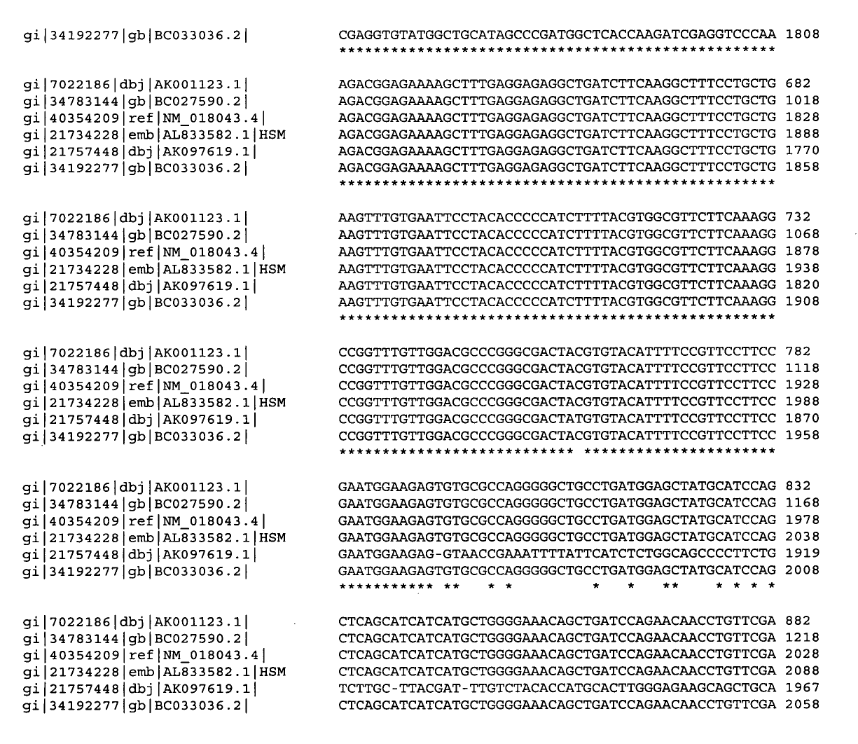

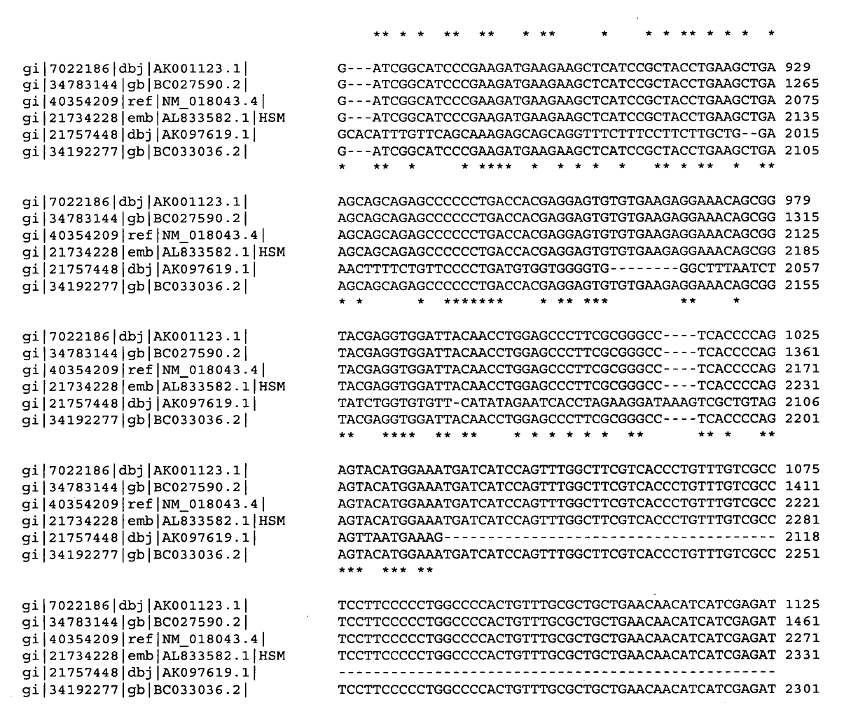

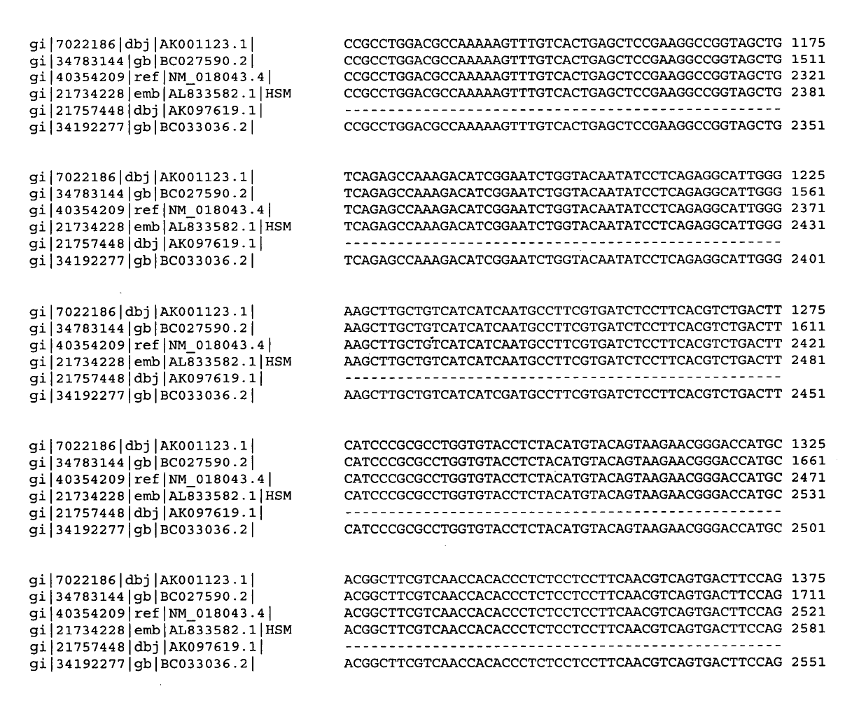

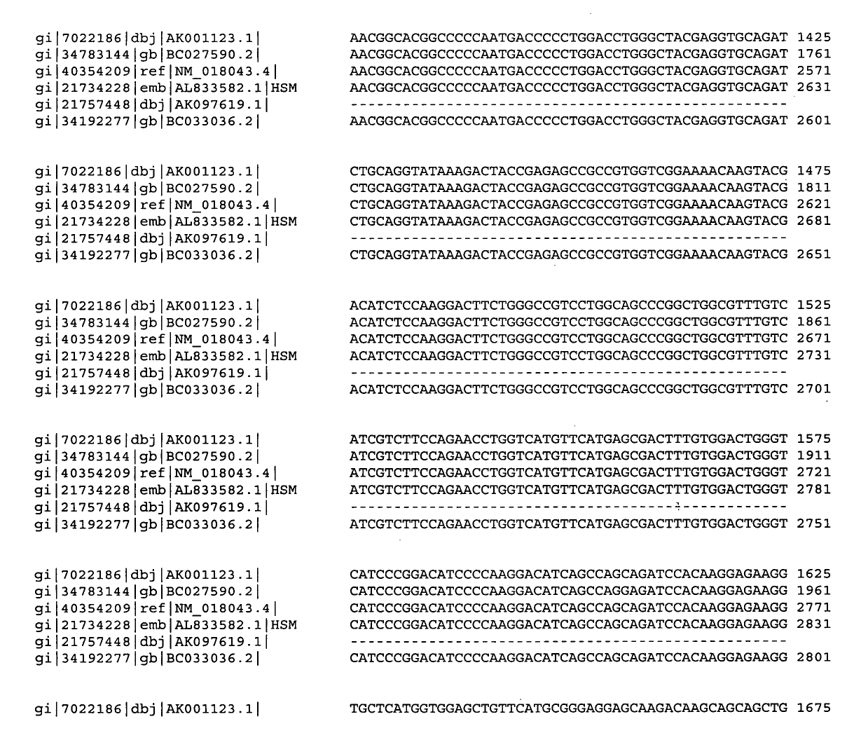

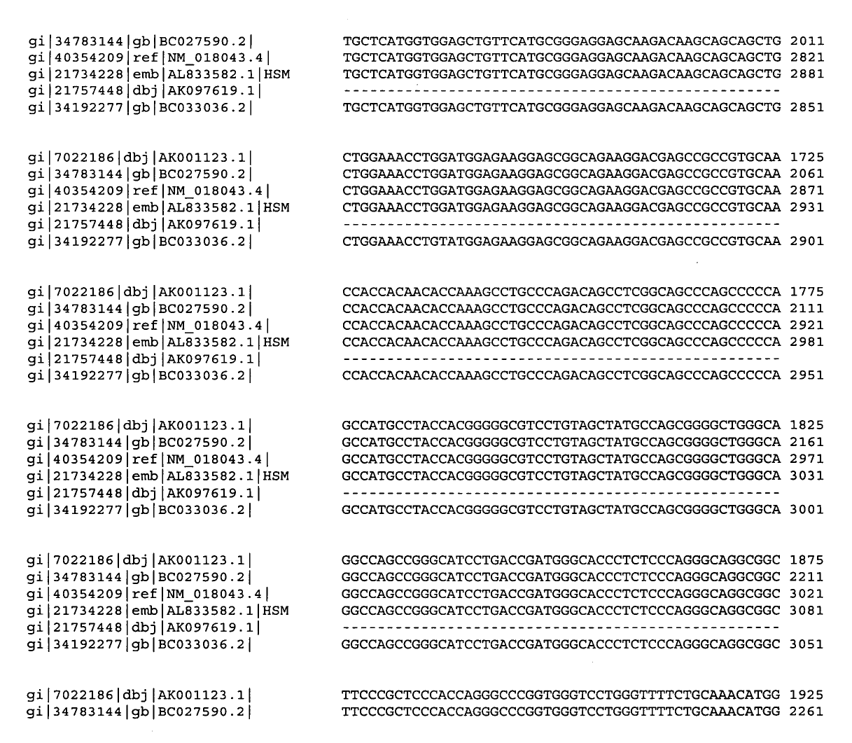

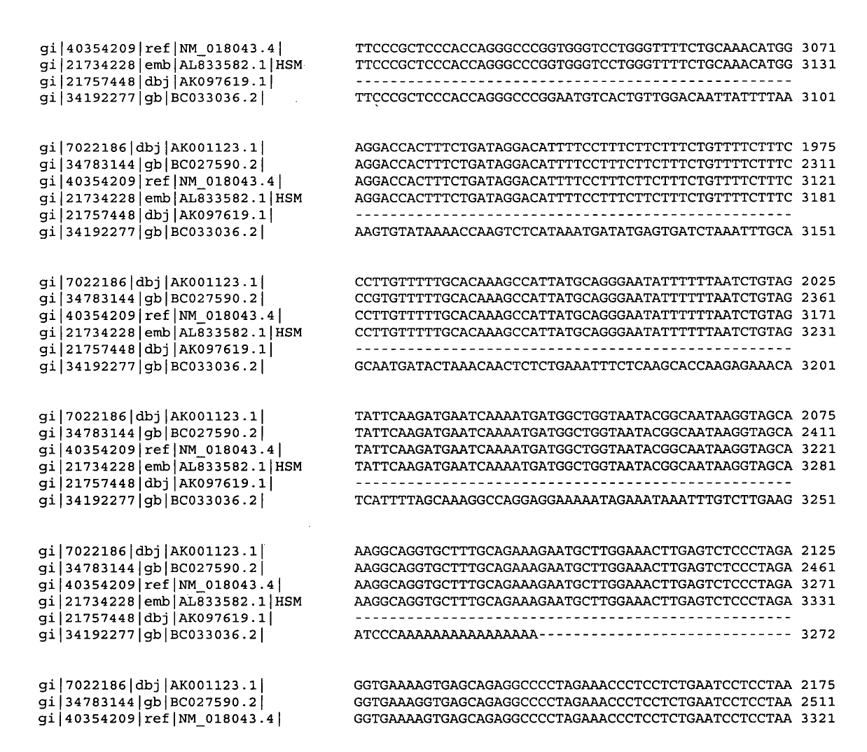

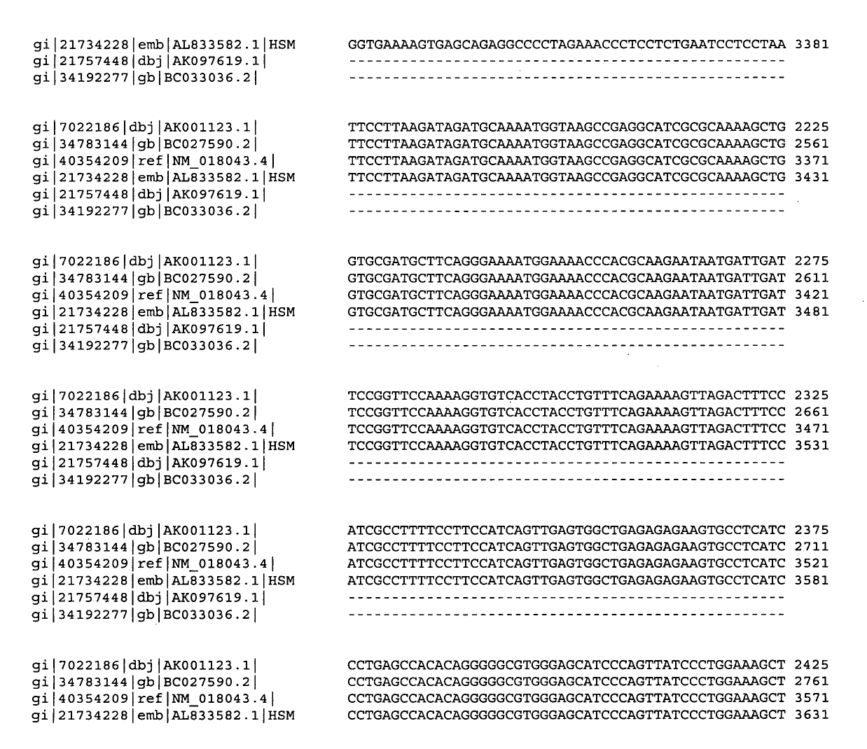

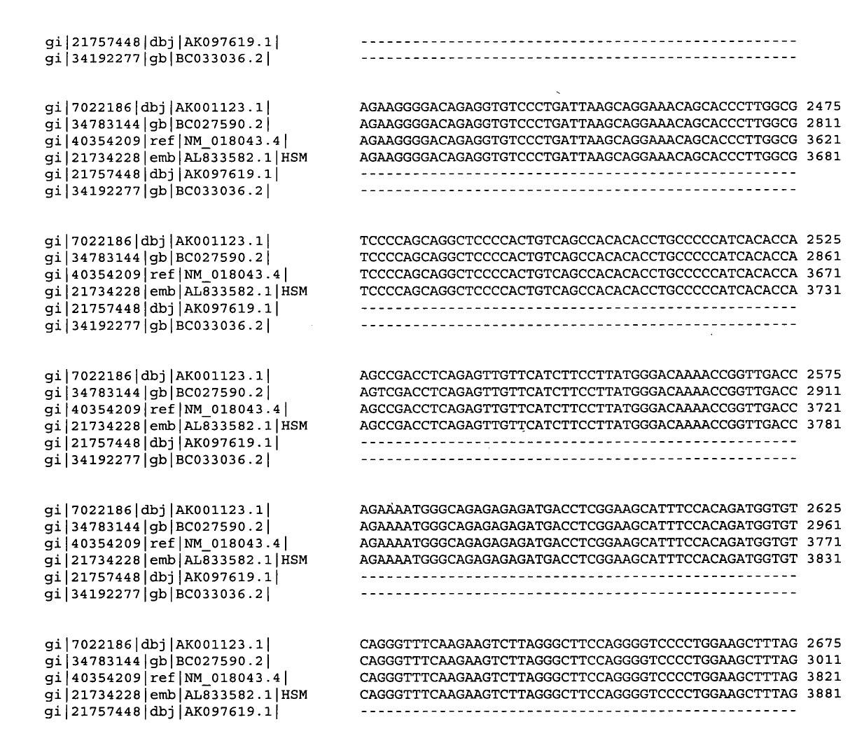

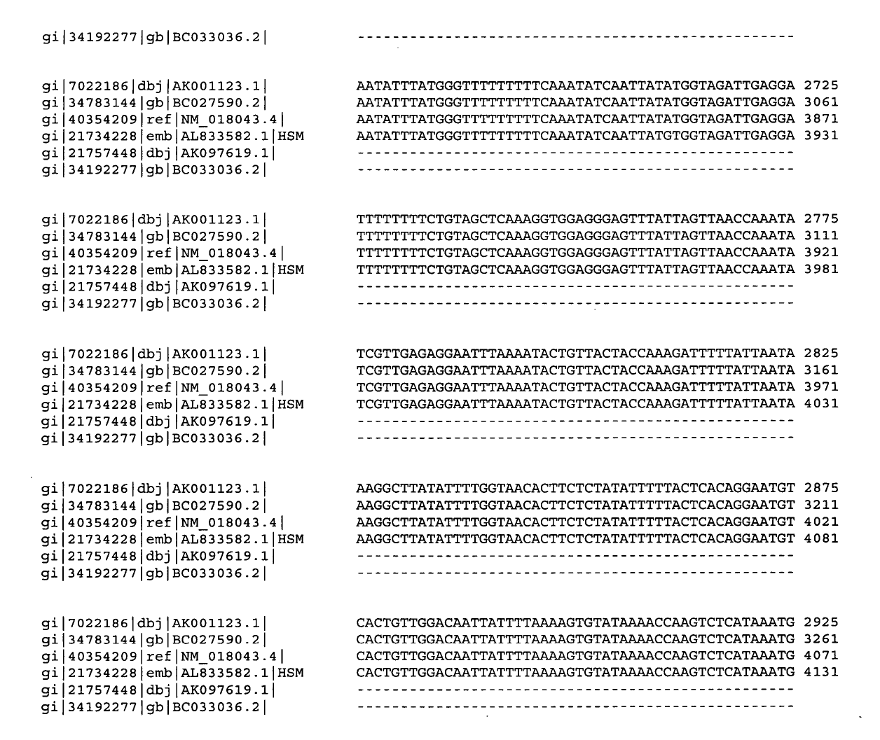

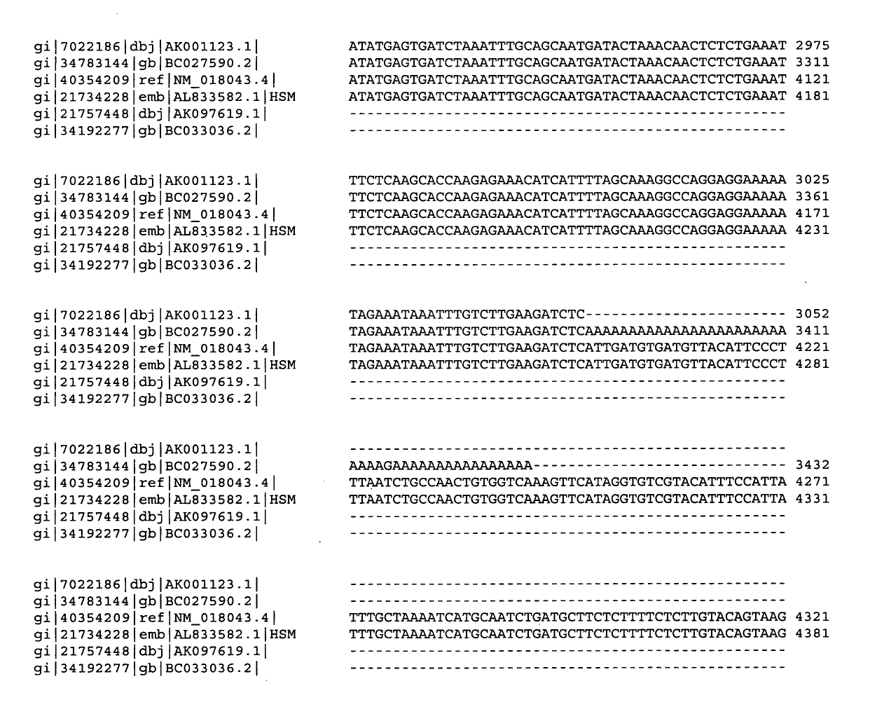

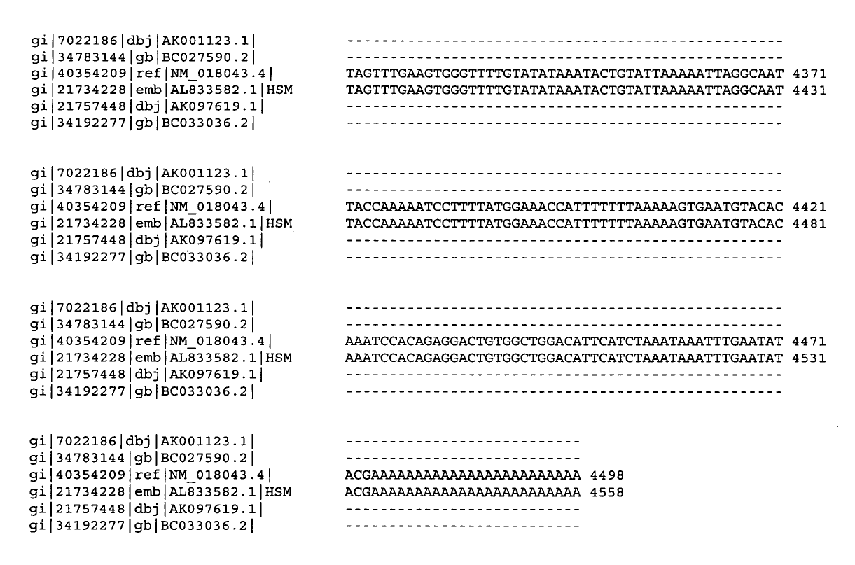

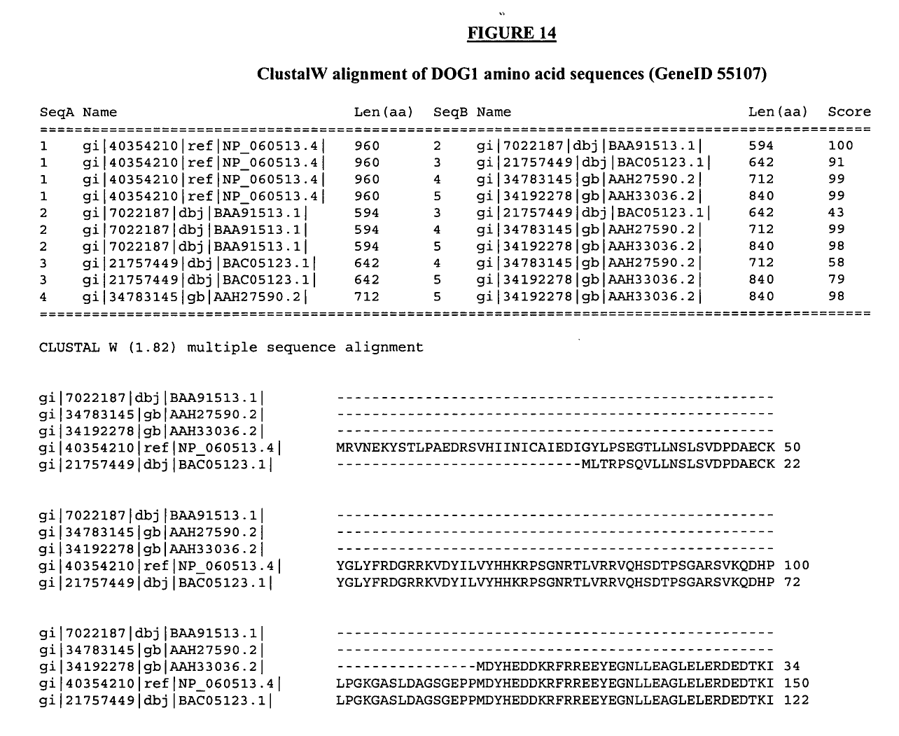

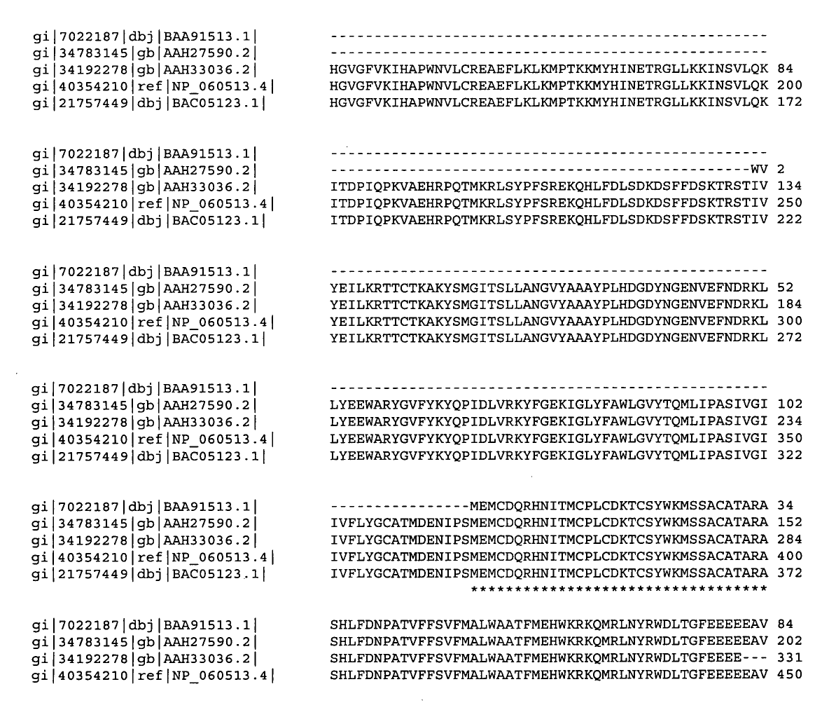

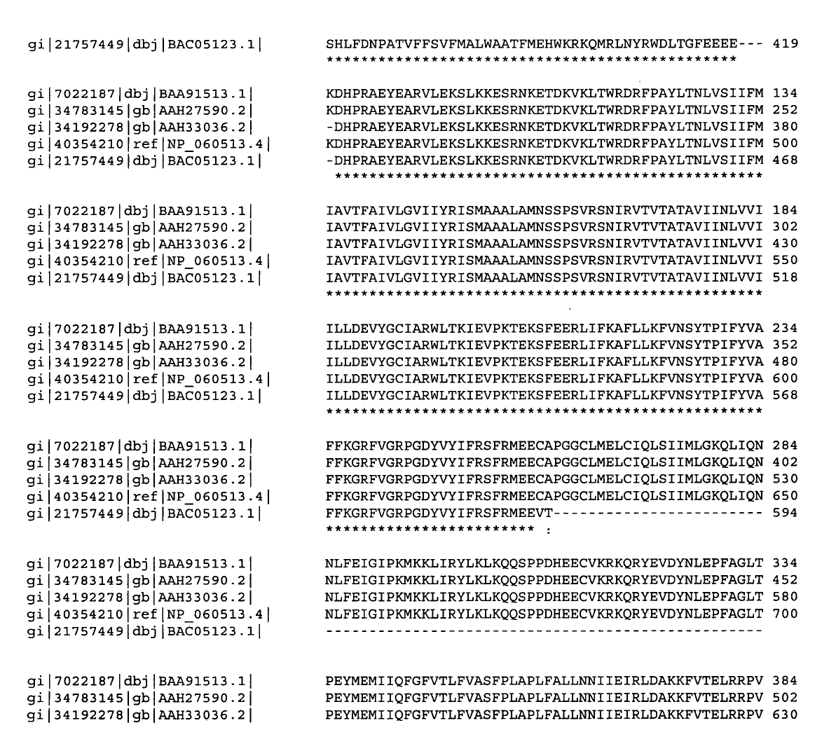

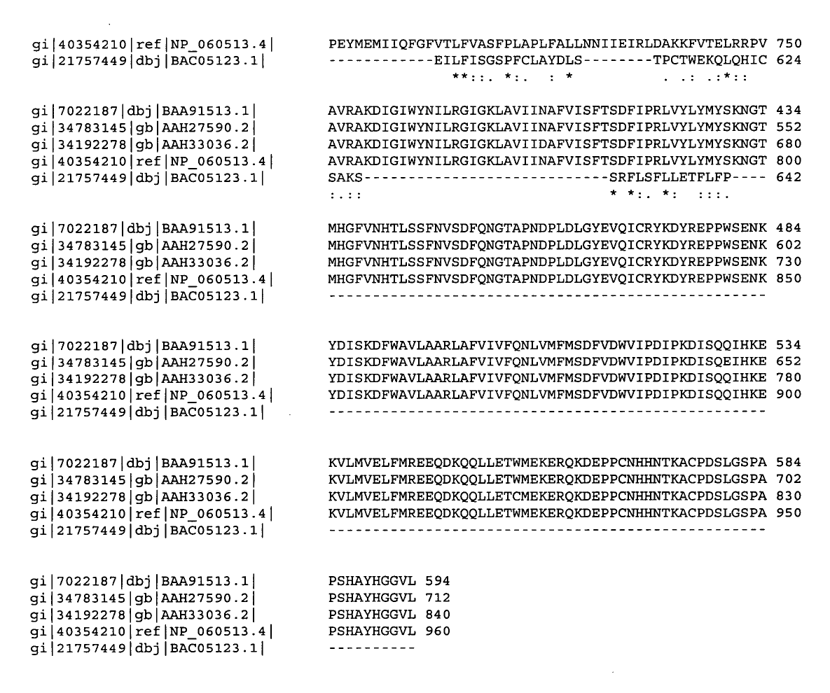

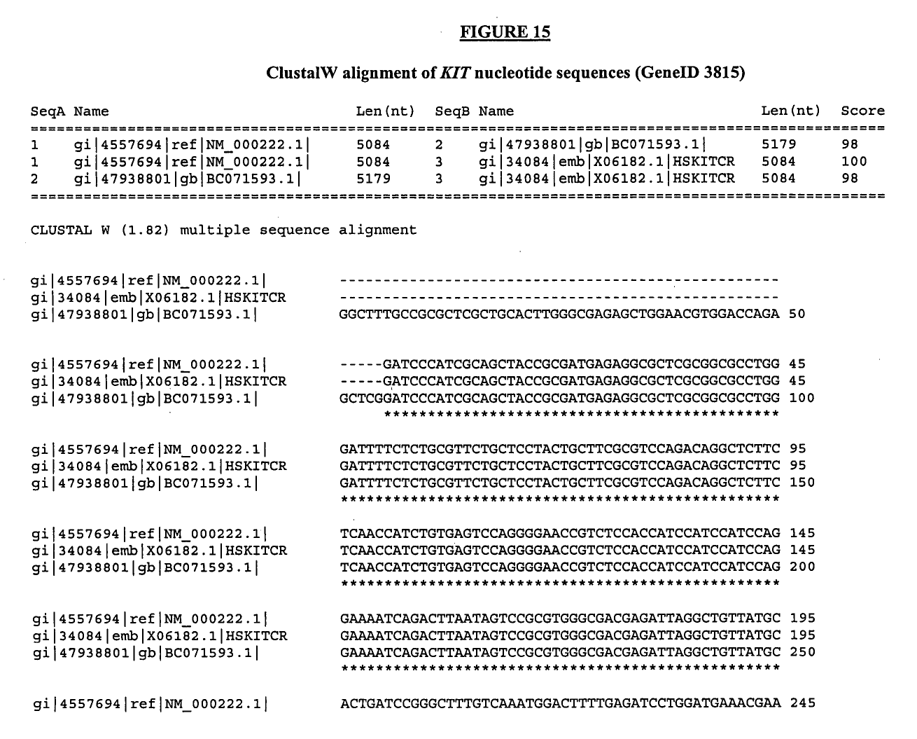

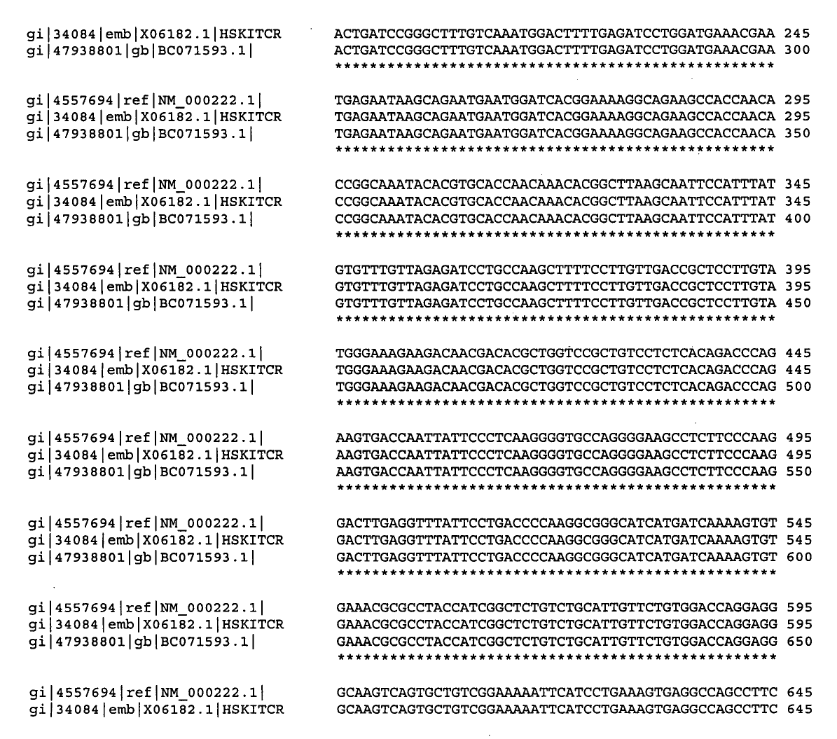

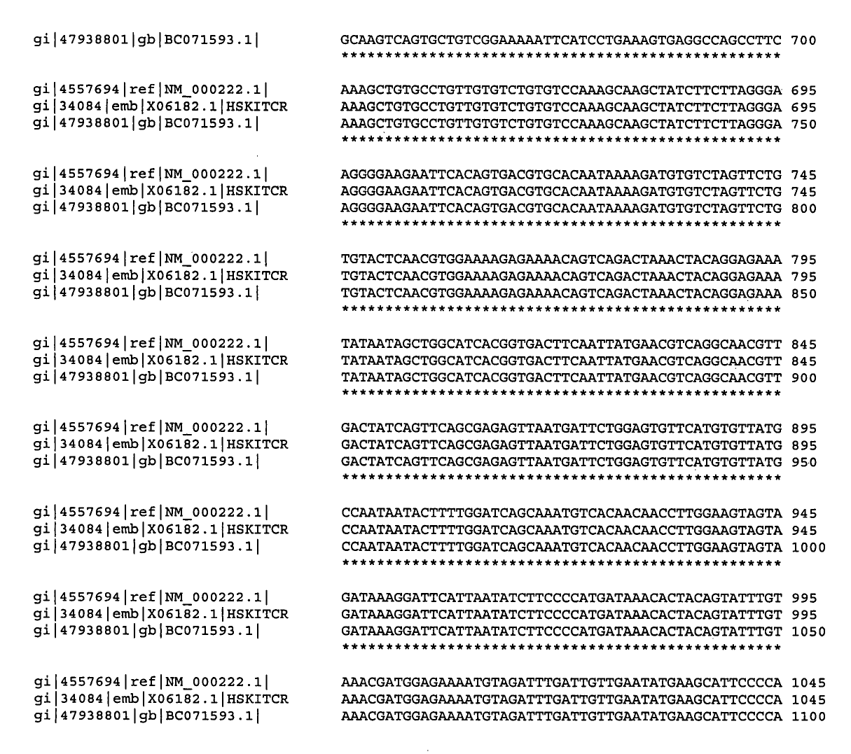

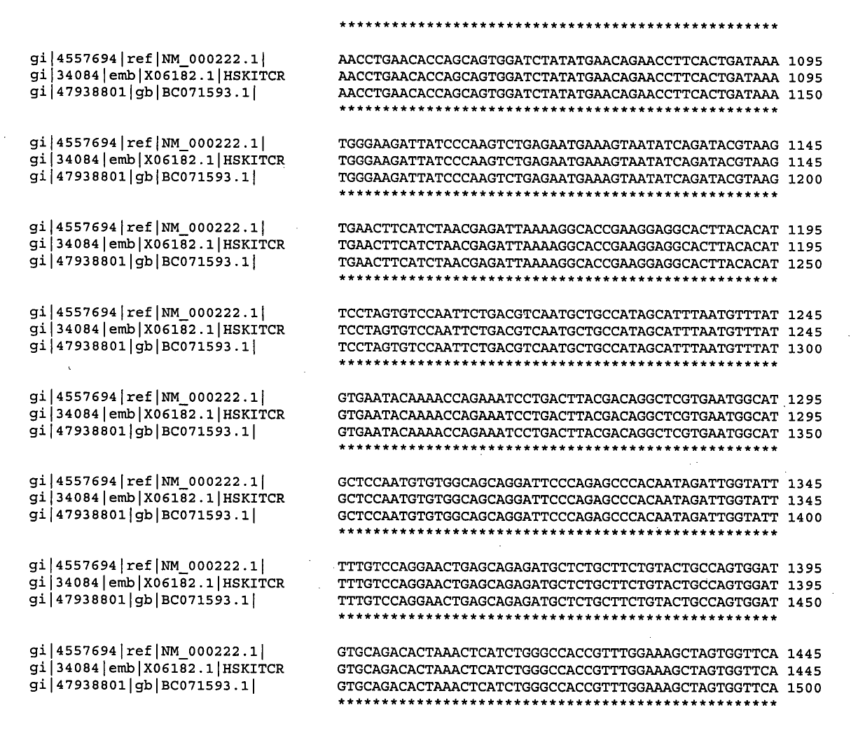

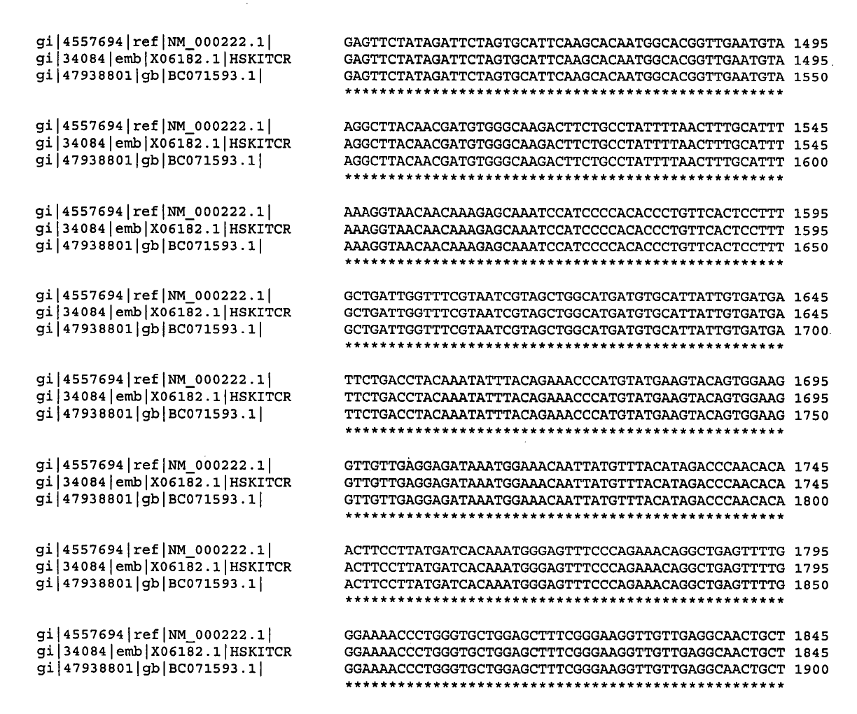

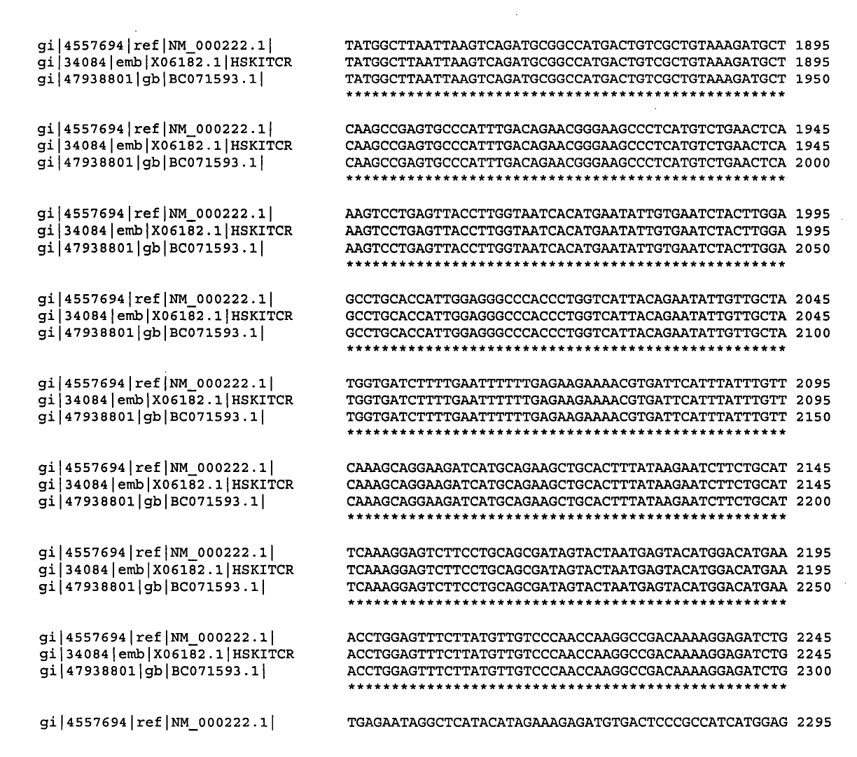

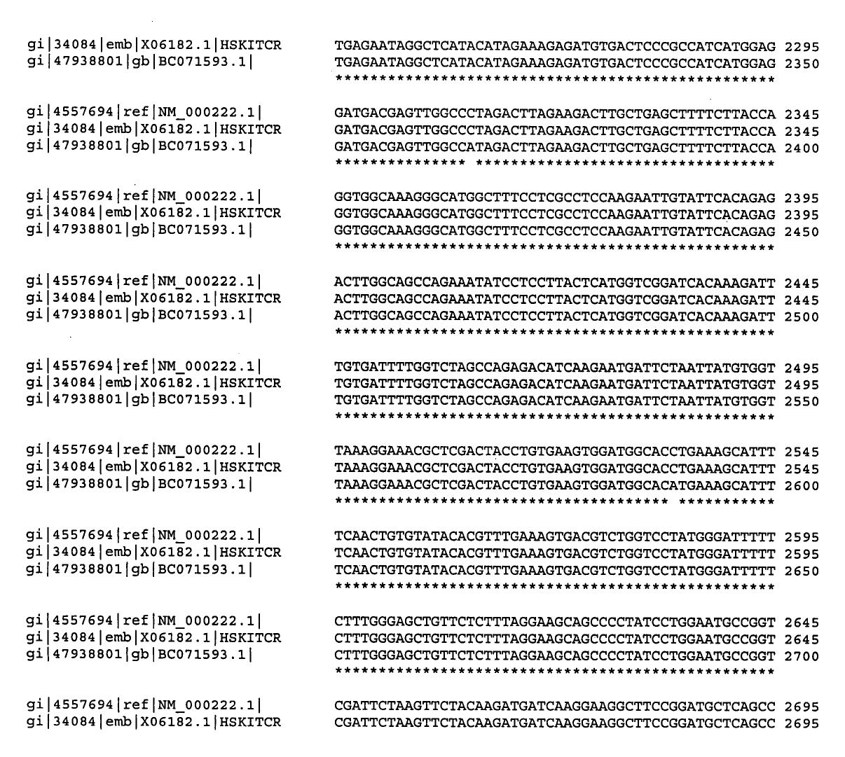

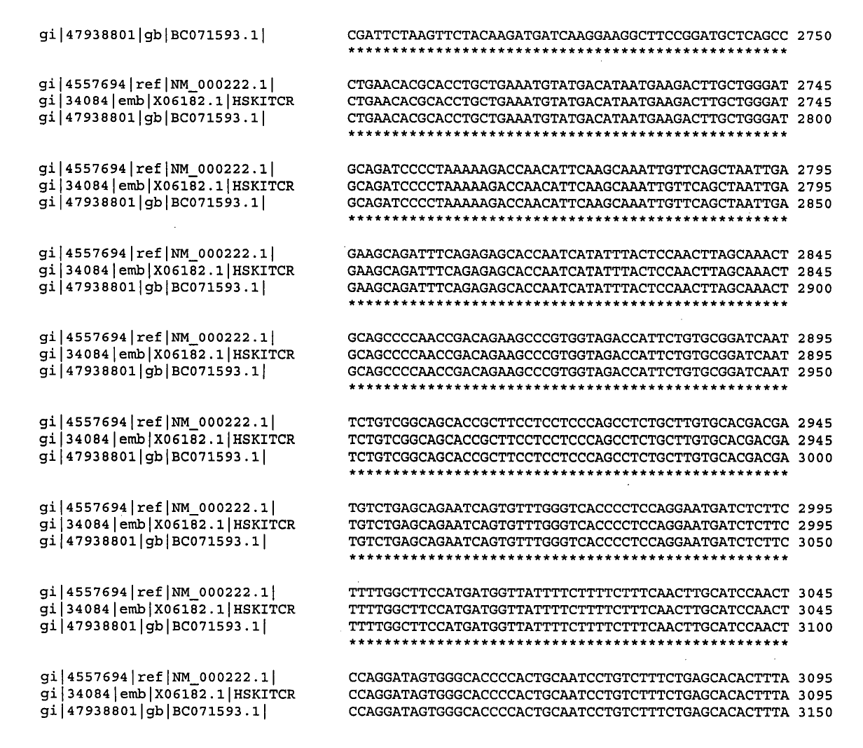

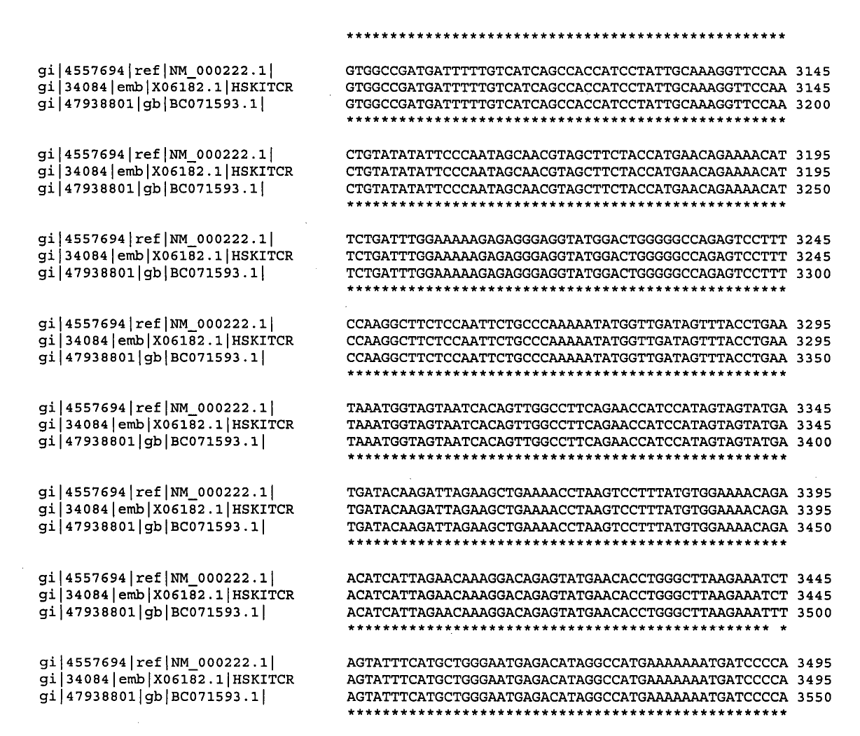

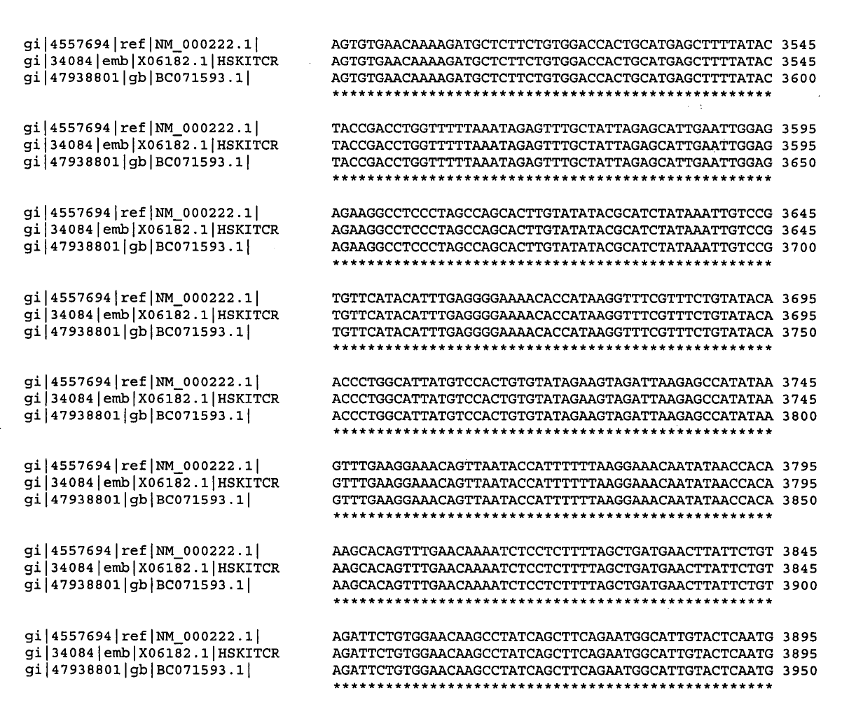

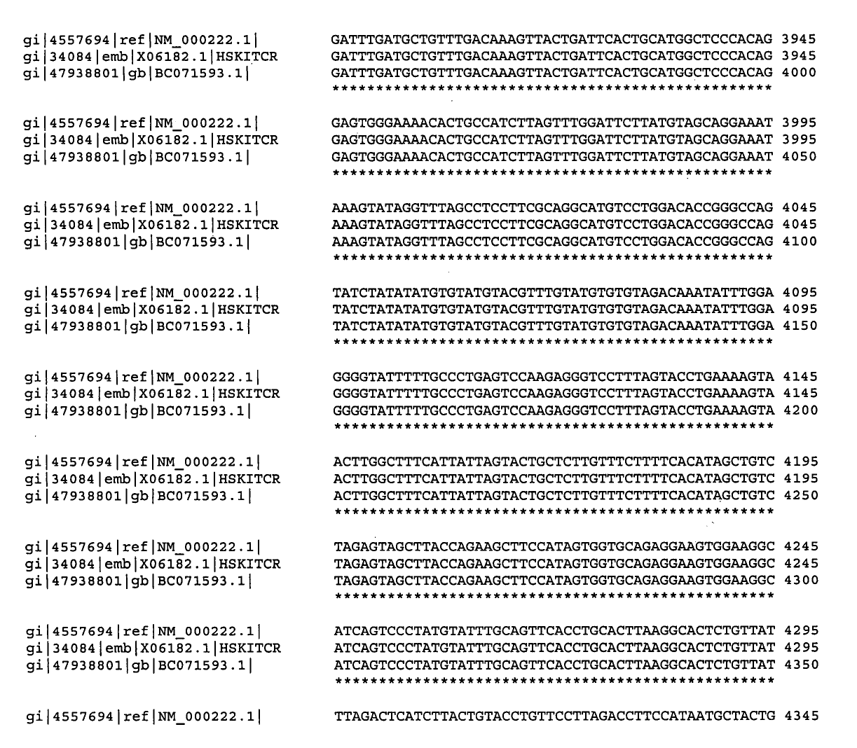

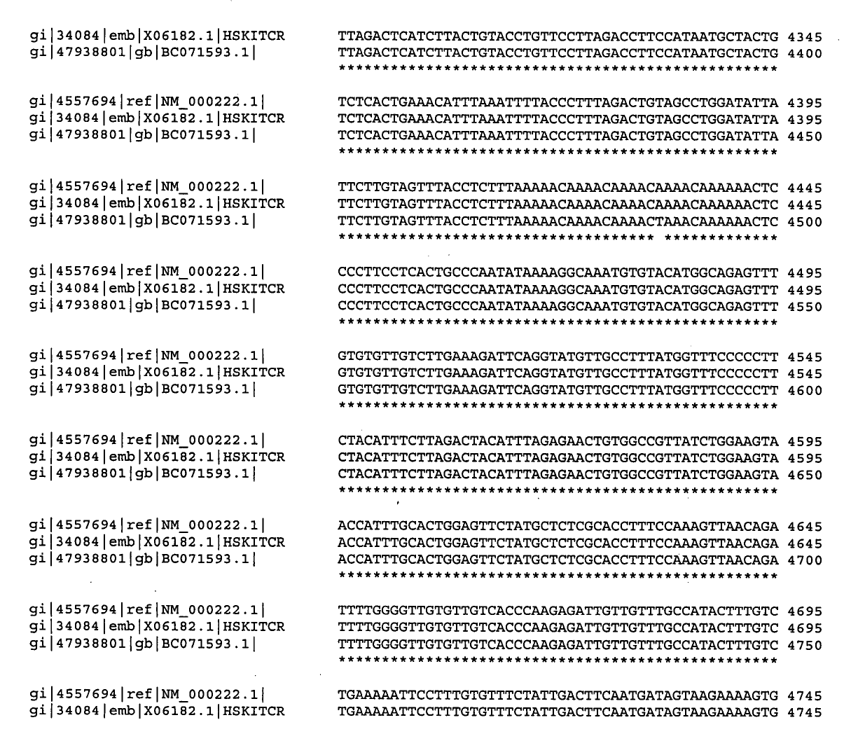

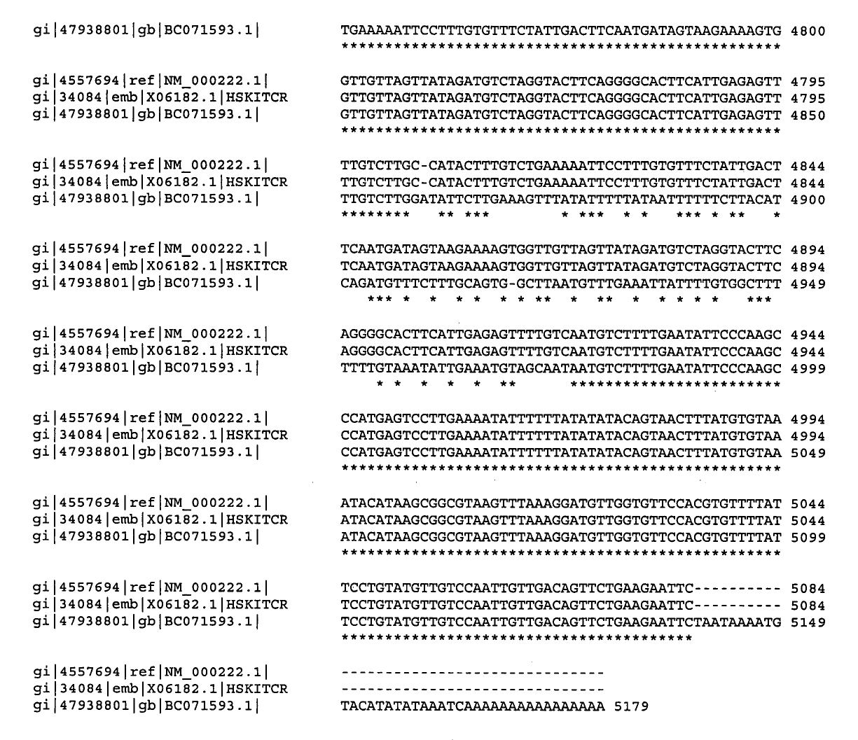

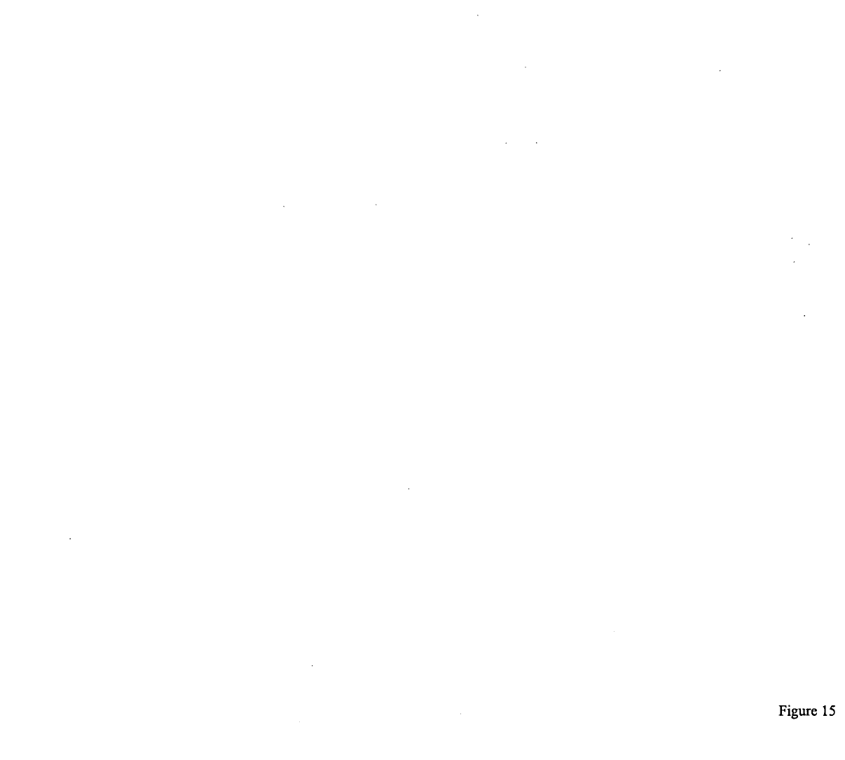

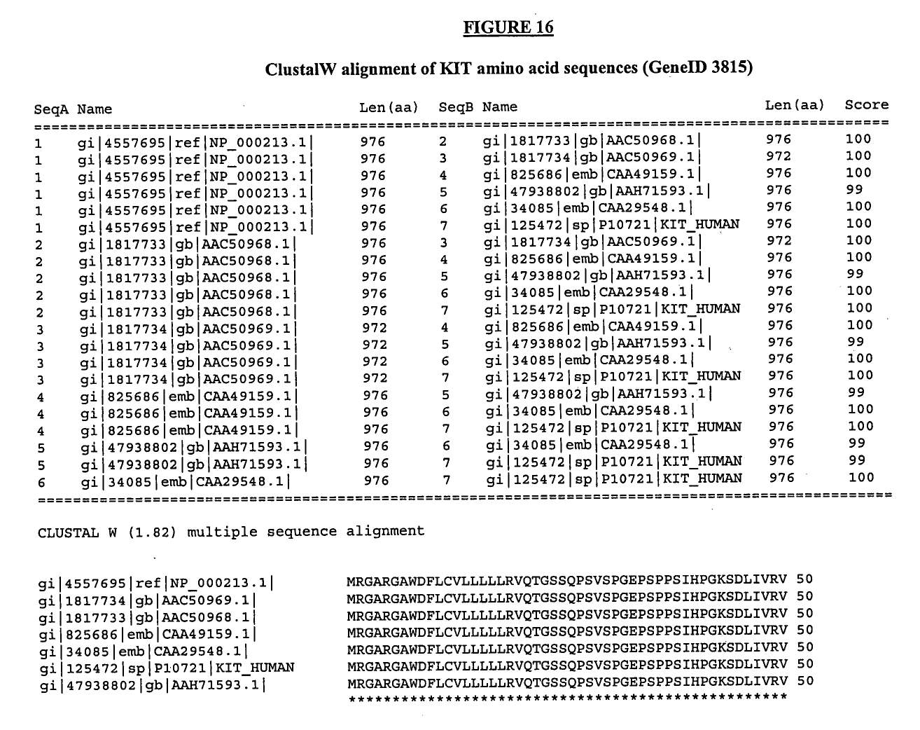

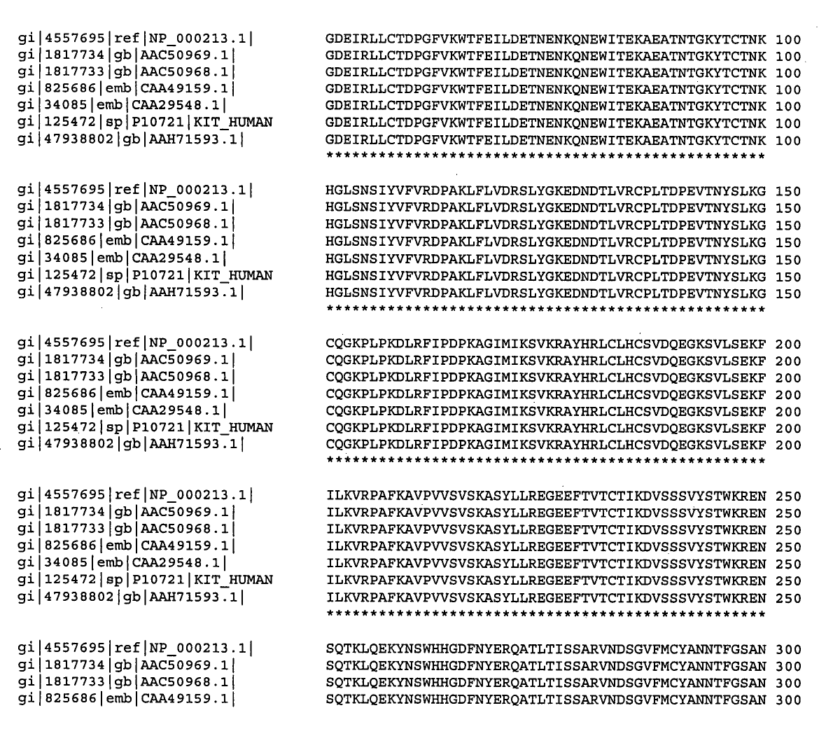

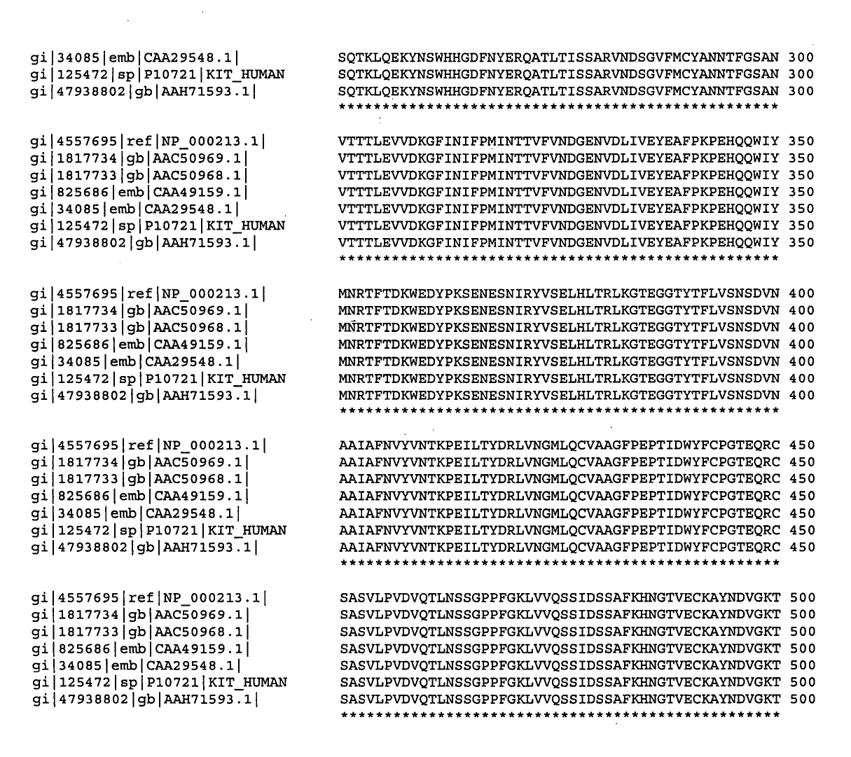

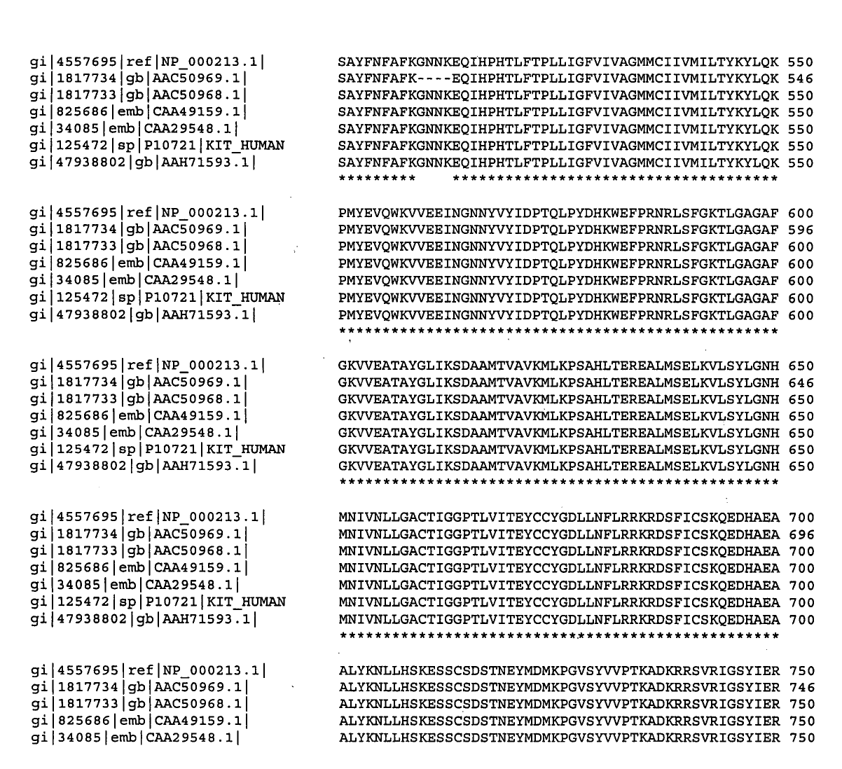

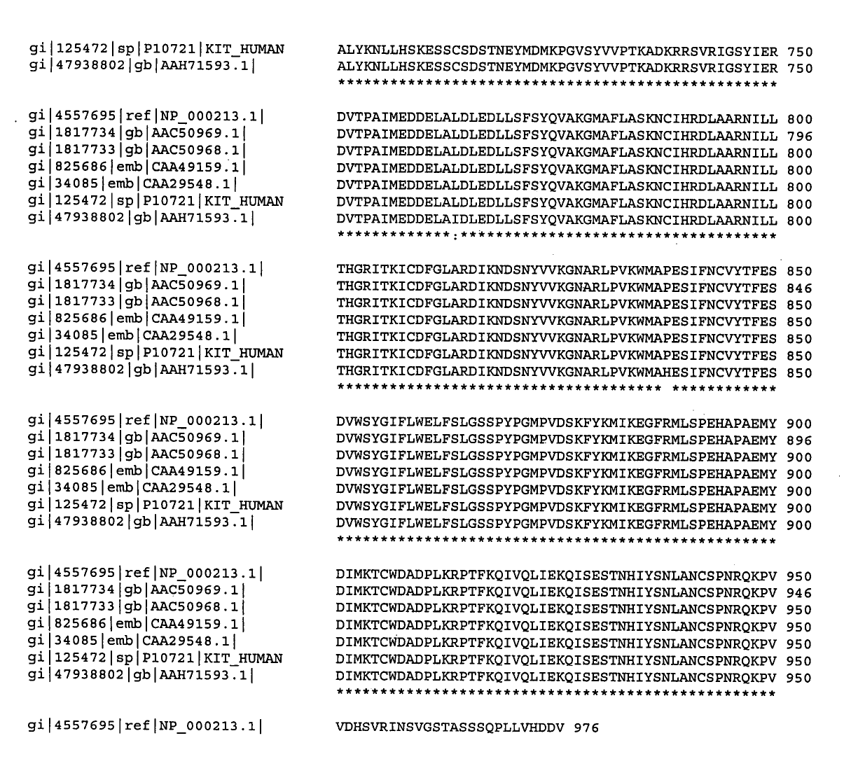

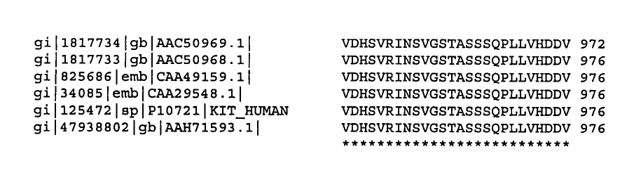

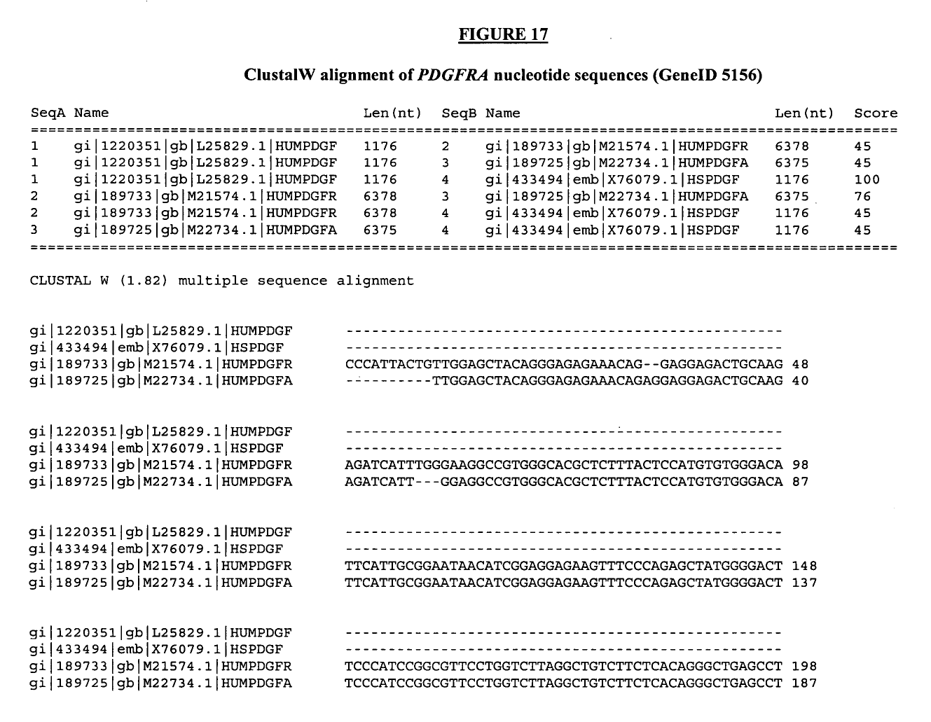

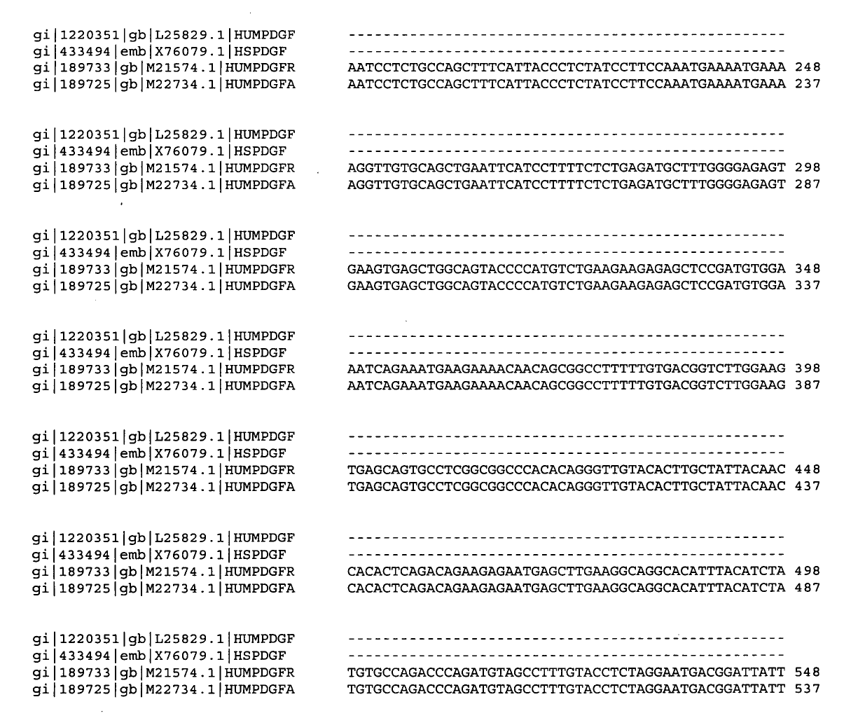

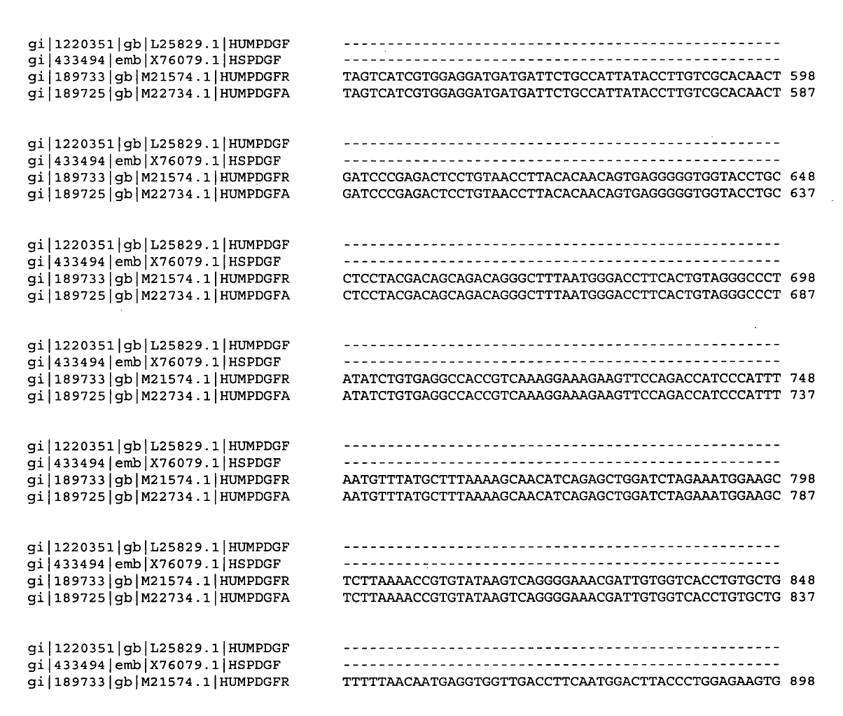

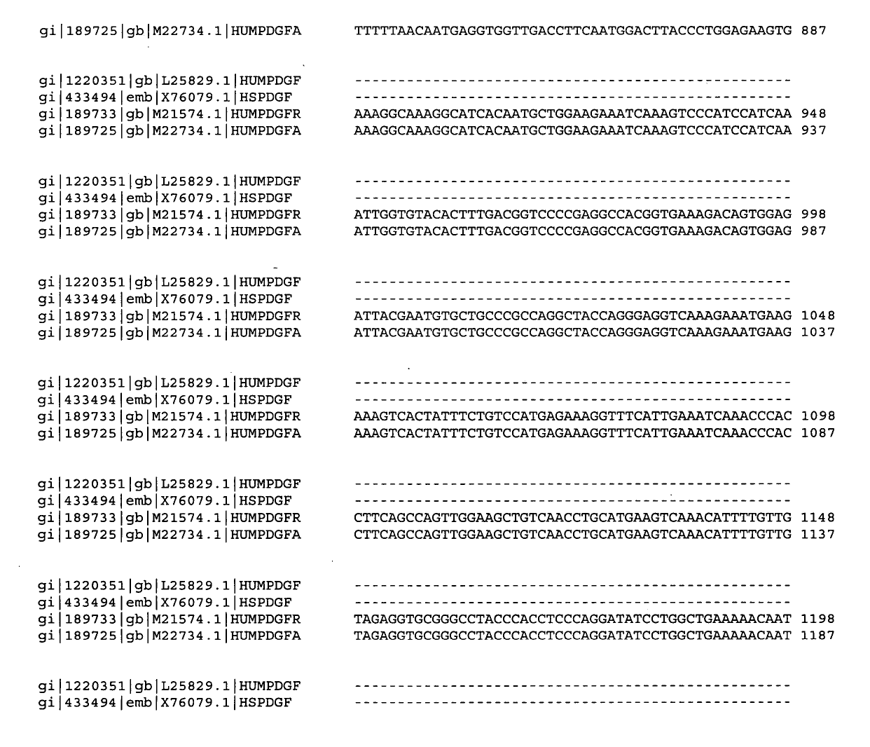

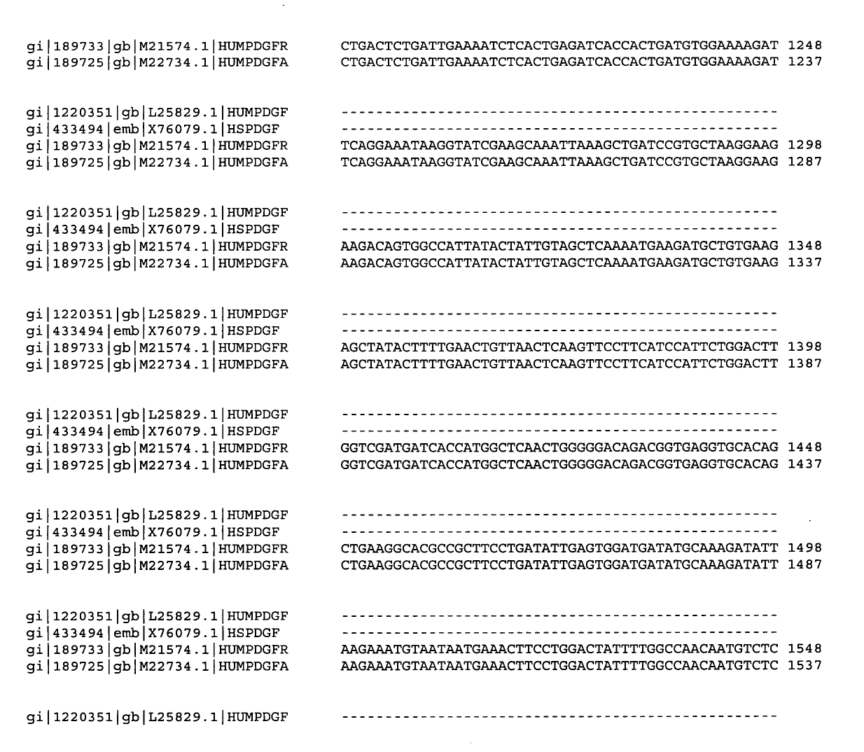

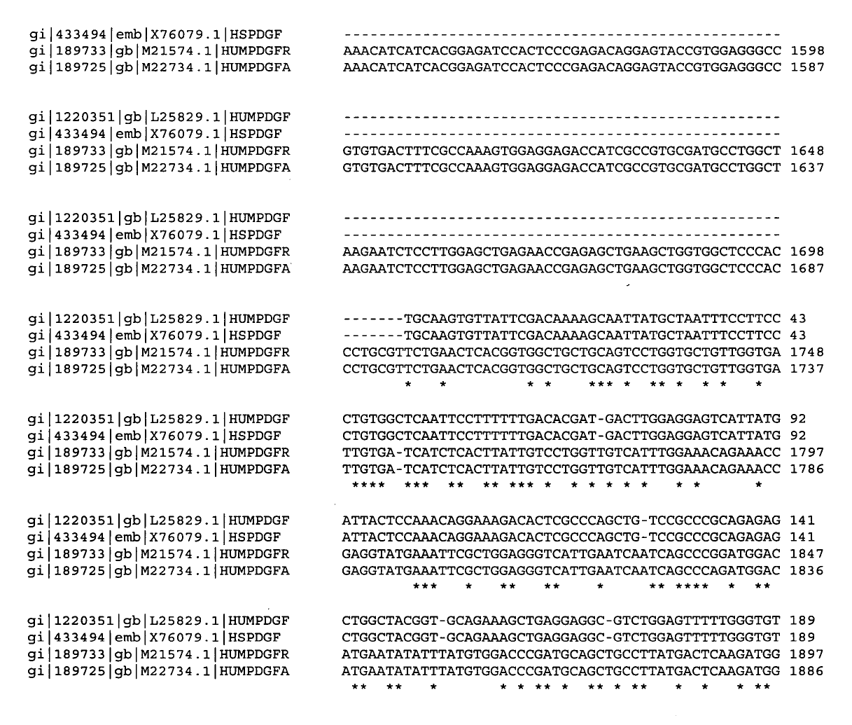

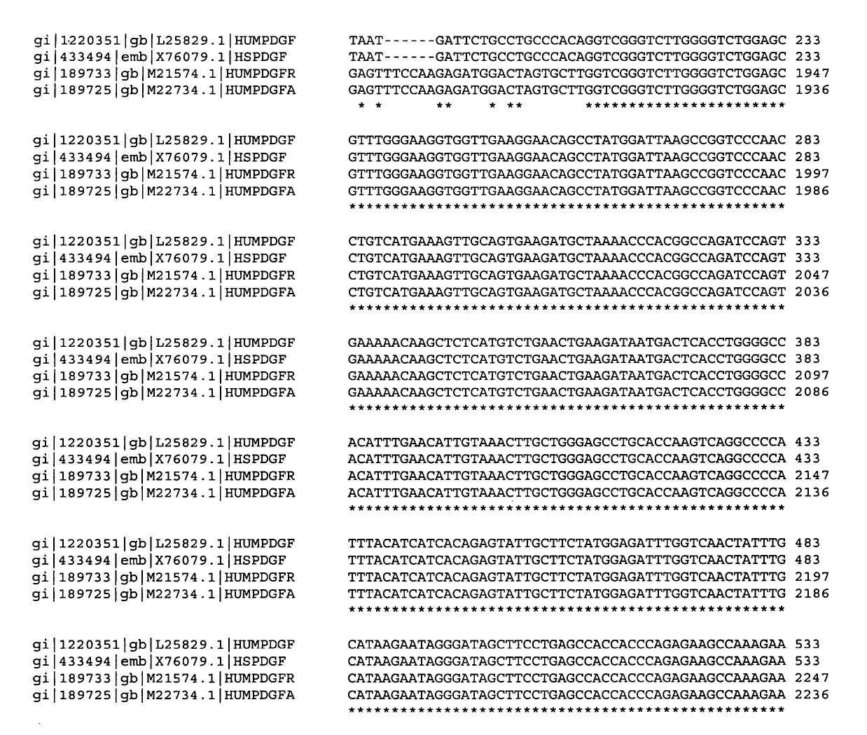

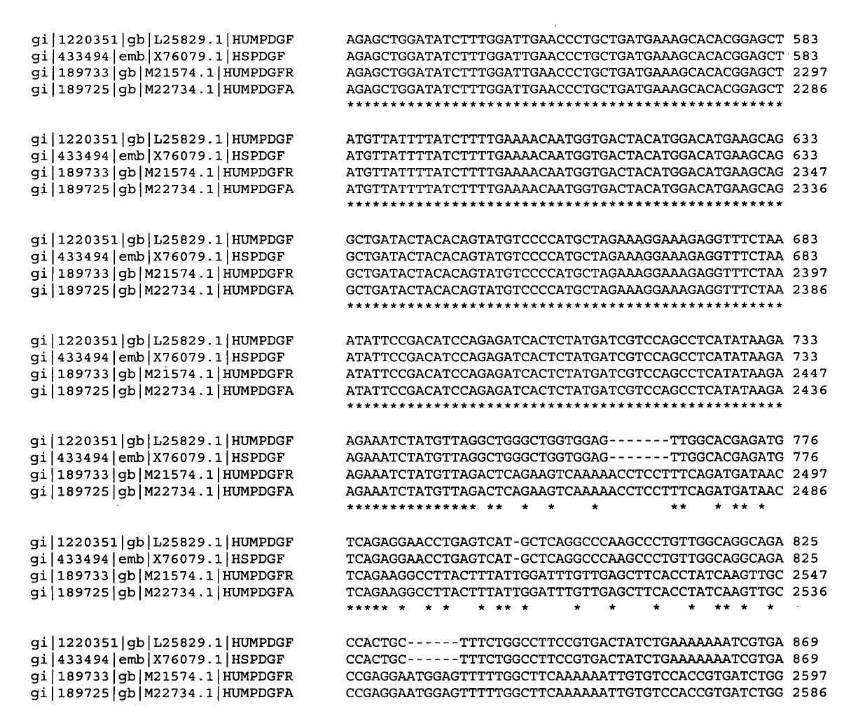

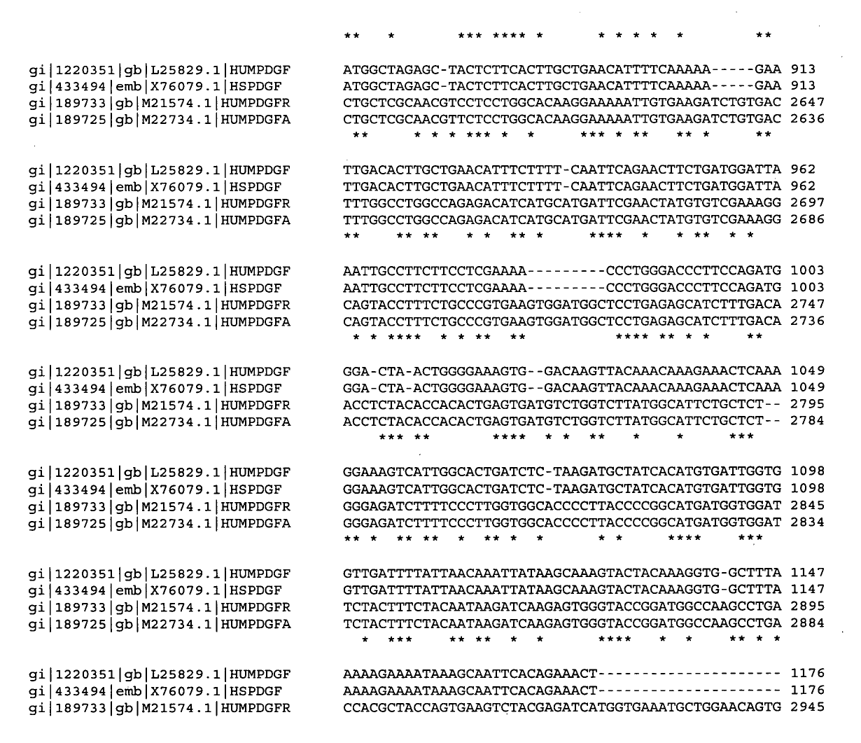

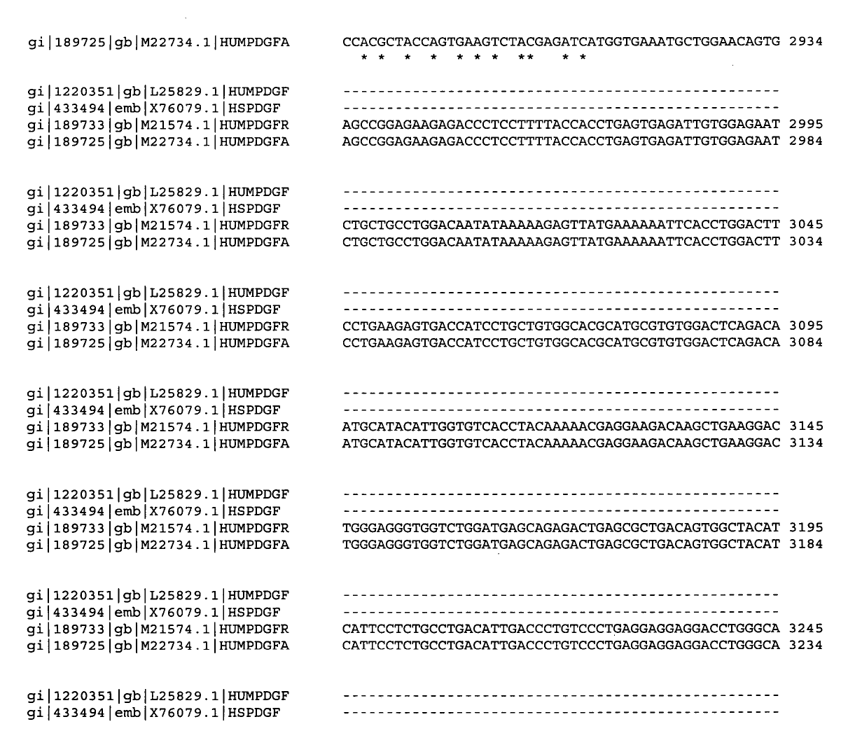

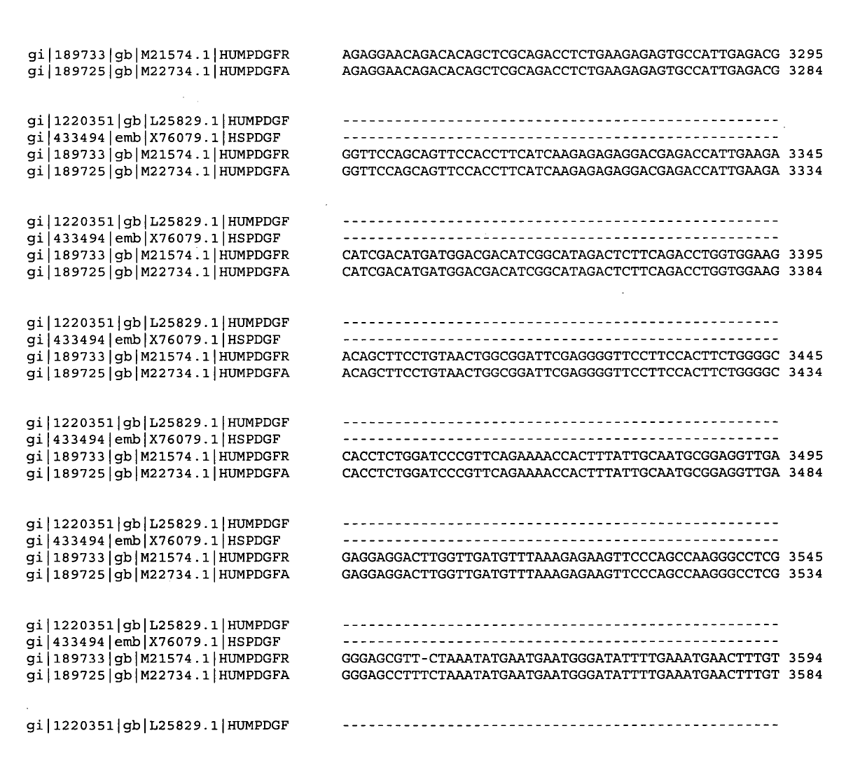

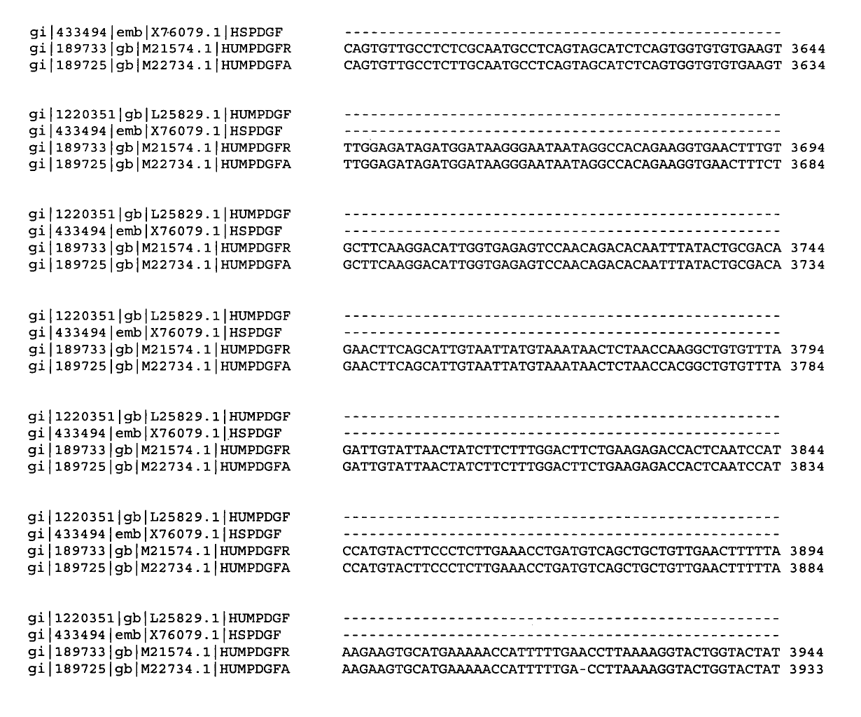

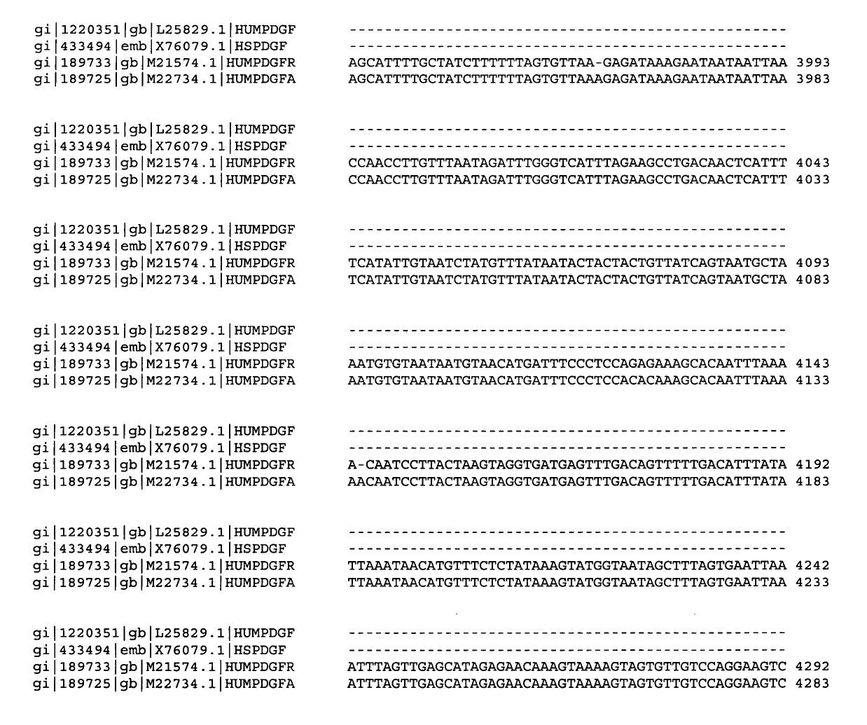

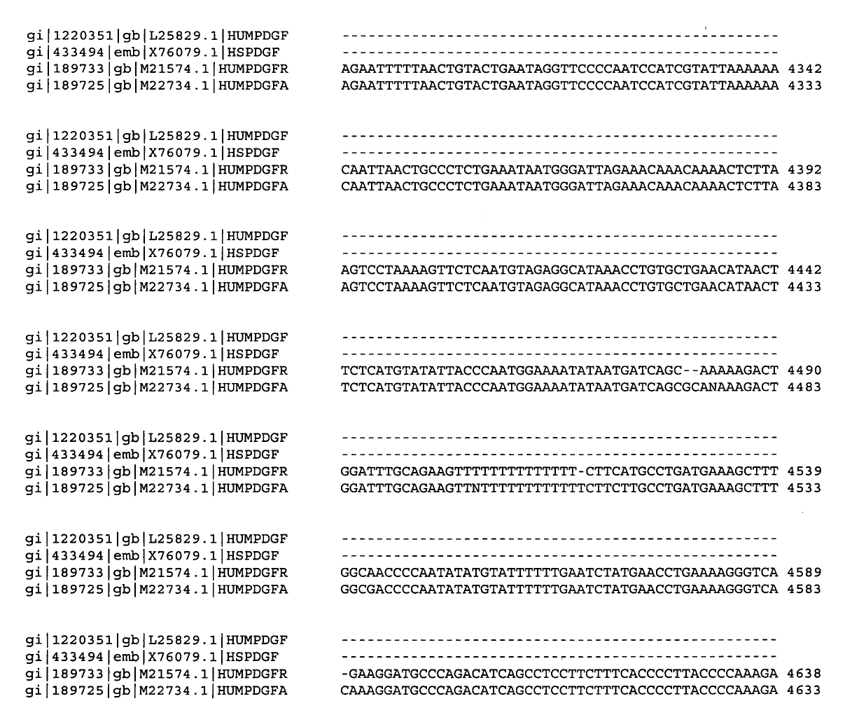

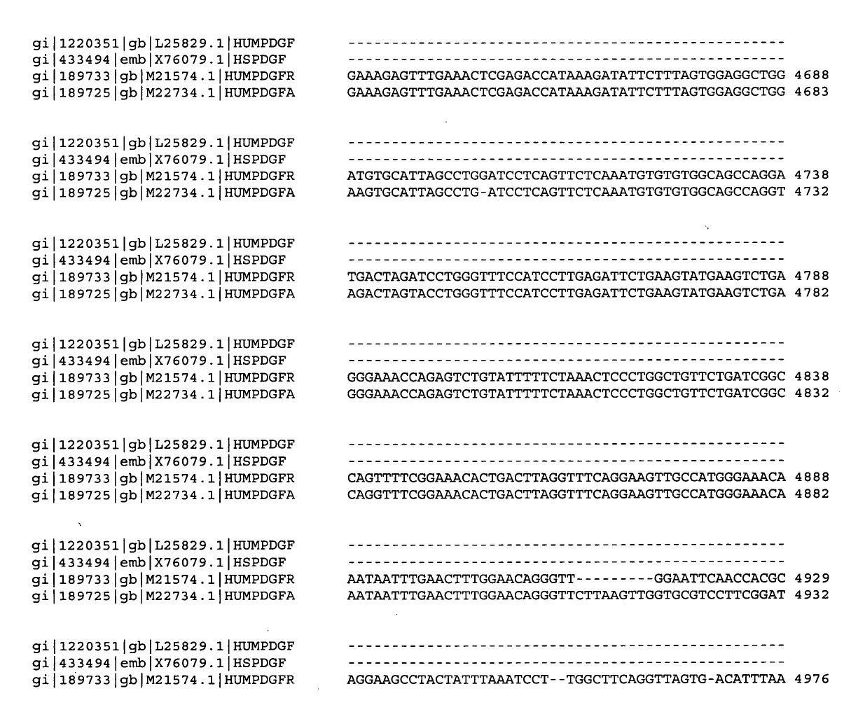

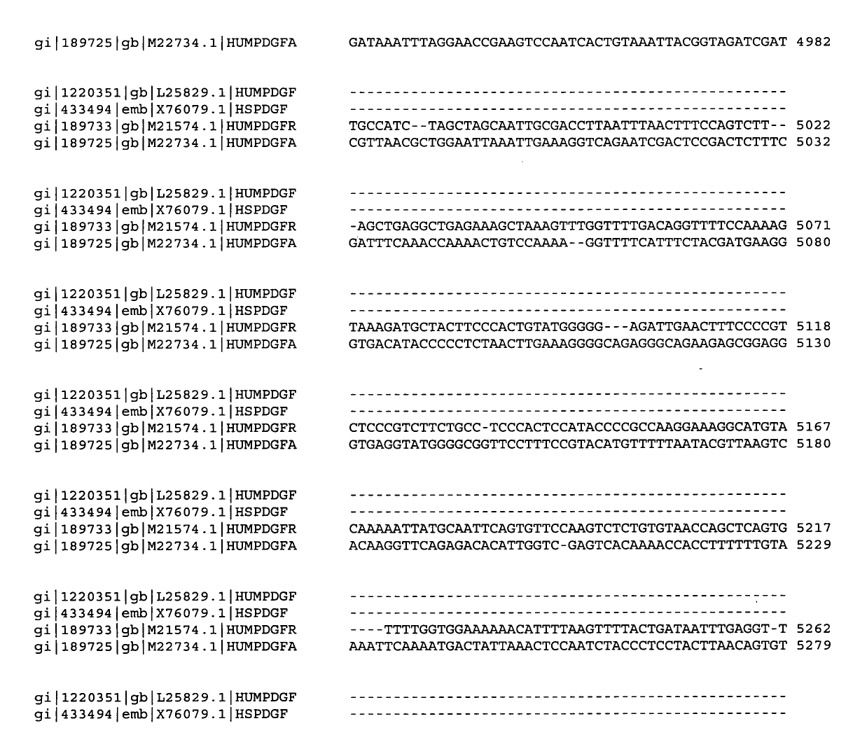

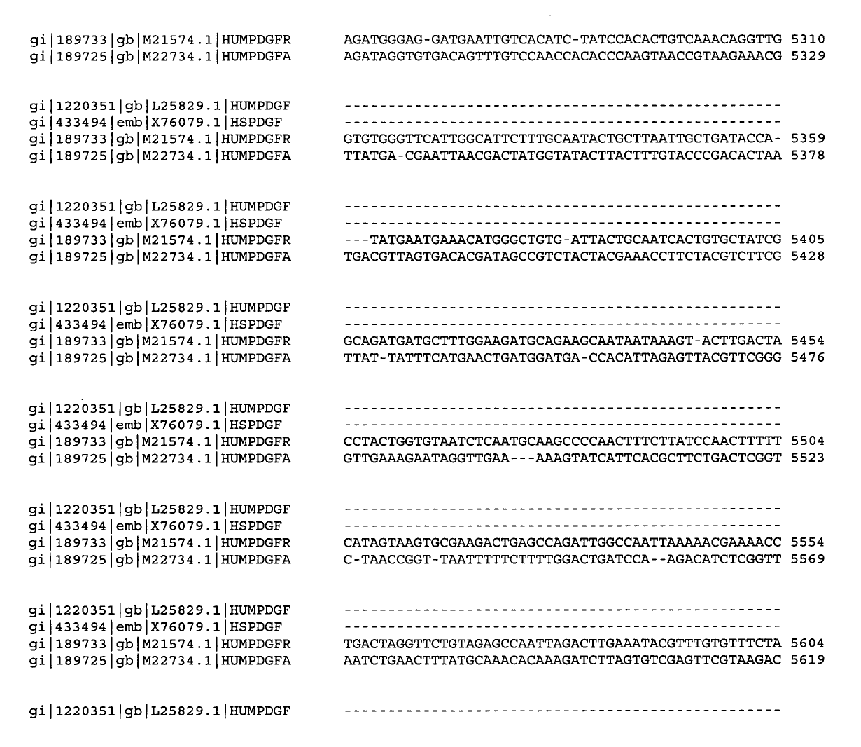

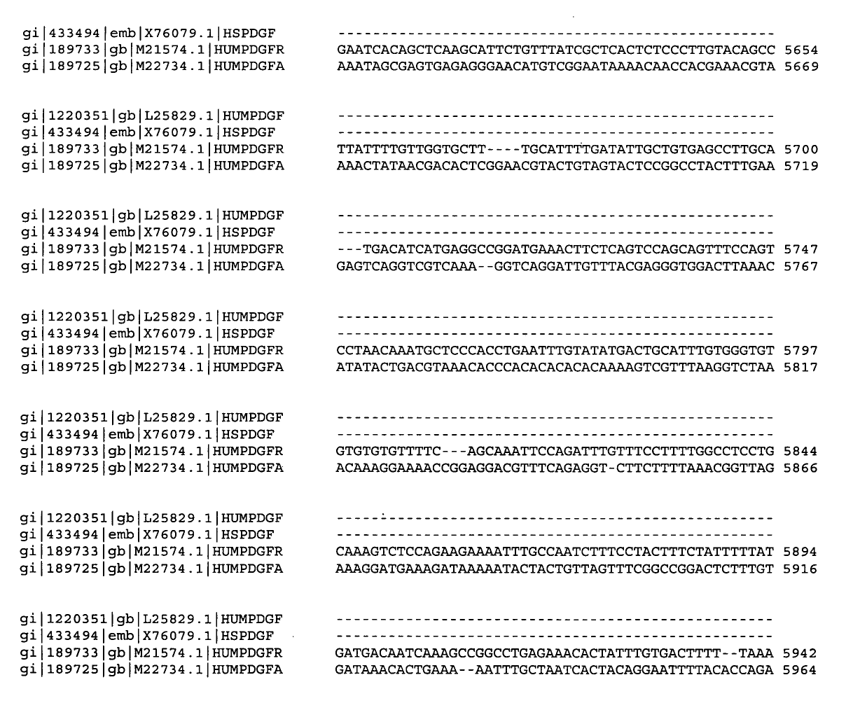

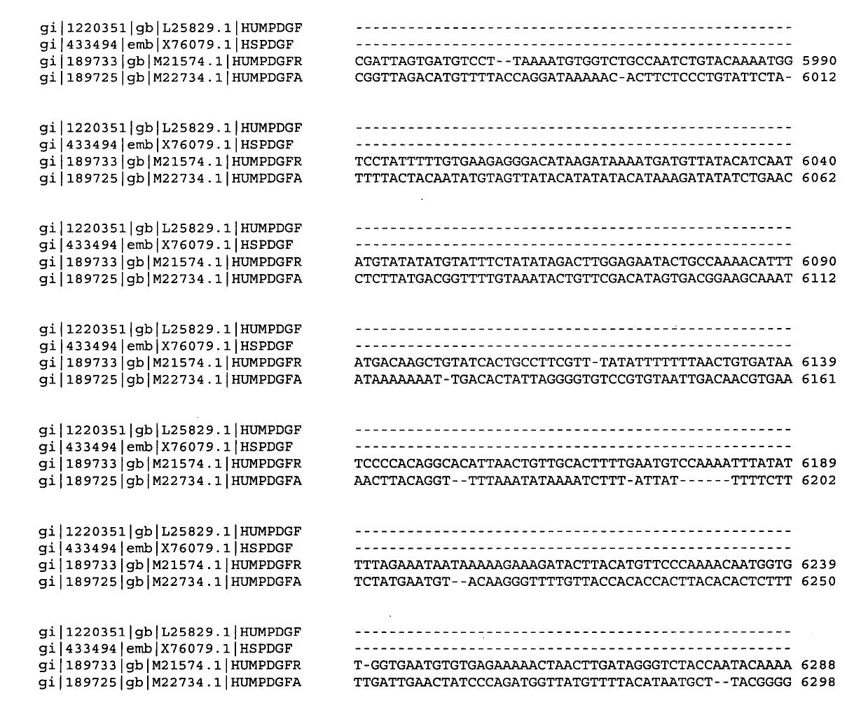

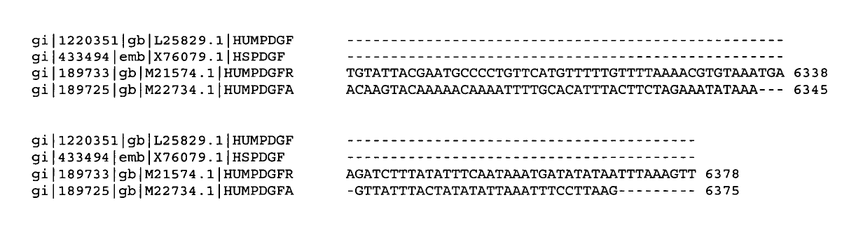

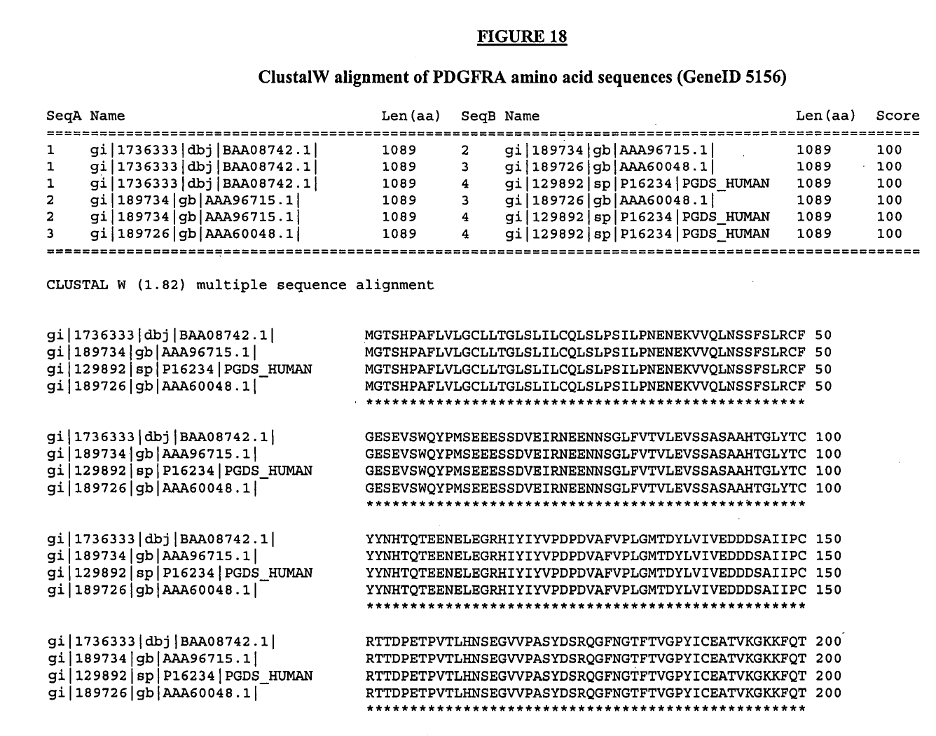

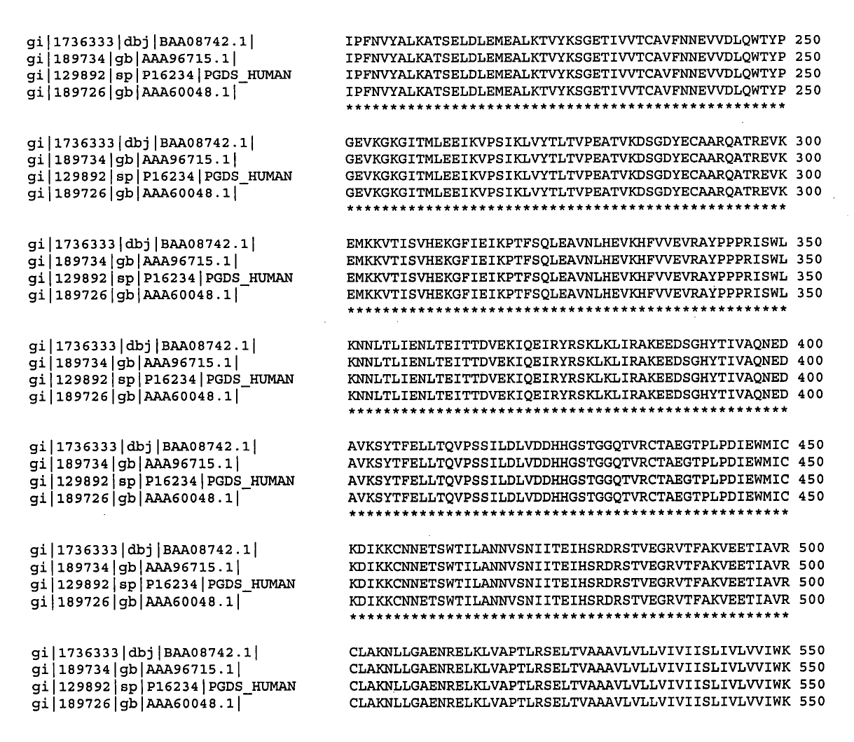

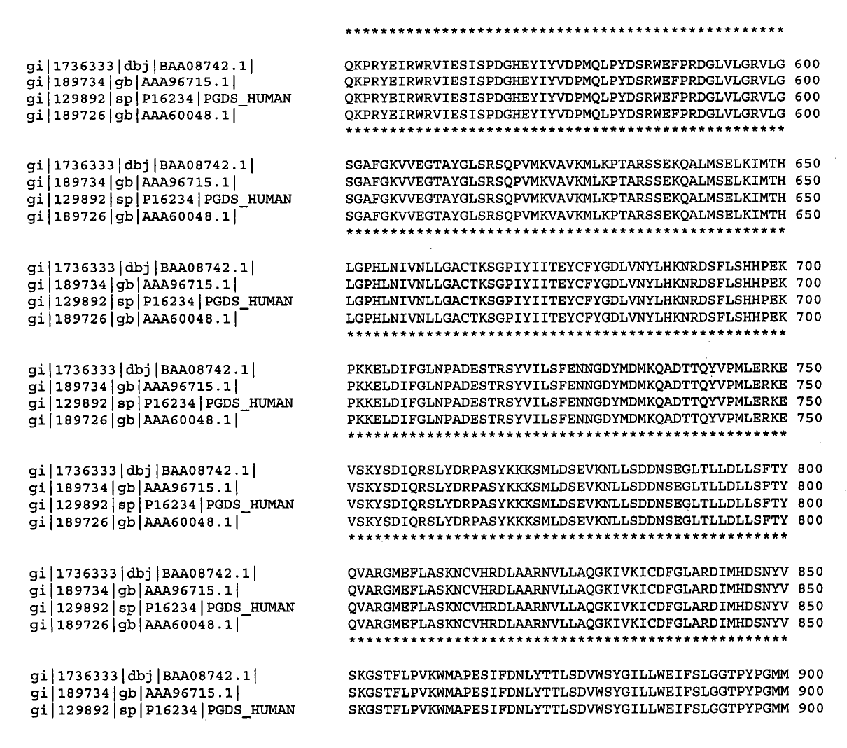

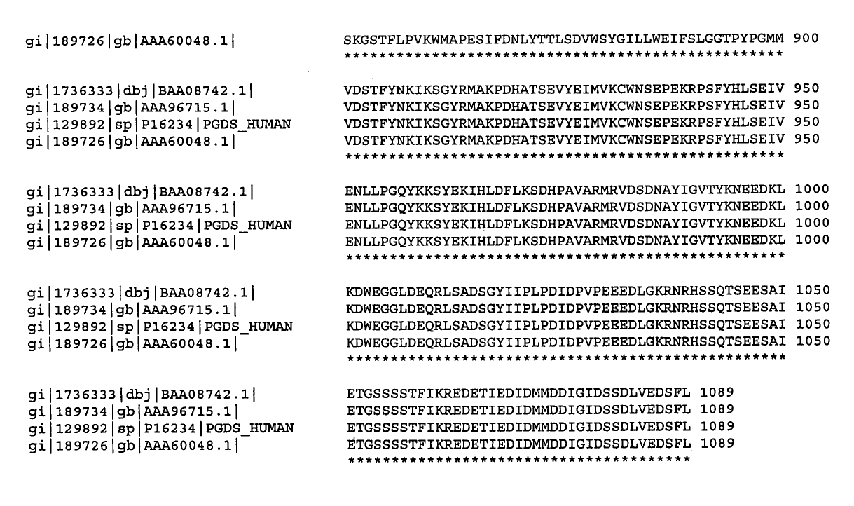

In these two TMAs, 136 of 139 scorable GISTs (97.8%) demonstrated immunoreactivity with DOG1 antiserum (see FIGS. 2 and 3, Table 1). The staining observed with DOG1 antisera appeared predominately localized to the plasma membrane (FIG. 4A). In some very strongly immunoreactive samples, the subcellular distribution of the staining could not be evaluated (FIG. 4B). Mast cells present in some of the samples, for example synovial sarcoma, were strongly immunoreactive as well (FIG. 4C), while the same samples showed only weak staining in the mast cells with KIT antibodies. These results were confirmed with in situ hybridization studies (FIGS. 5 and 6). Unexpectedly, DOG1 antisera stained all 8 scorable PDGFRA-mutant GISTs (1 case from the first TMA and 7 cases from the second TMA), while the KIT antibody staining was weak in 3 of these cases and negative in the remaining 5 (Table 1). These findings were further extended by in situ hybridization with PDGFRA (FIG. 6). PDGFRA expression was predominately, but not exclusively, present in the PDGFRA-mutant GISTs. 5 of 6 (83%) scorable PDGFRA-mutant GISTs were positive for PDGFRA ISH (FIGS. 2 and 3, Table 1). In contrast, only 10 of 70 (14%) KIT-mutant and KIT-wildtype GISTs were positive for PDGFRA ISH (Table 1). Correlation of KIT ISH with KIT immunohistochemistry was good, with the ISH signal detectable in almost all immunopositive cases (FIG. 2). However, a difference was seen in the PDGFRA-mutant GISTs with regard to KIT expression. Three cases were immunopositive for KIT, but only one case was positive by KIT ISH. Hierarchical clustering analysis of IHC and ISH data was performed as previously described²⁵. Among these parameters—KIT IHC, KIT ISH, DOG1 IHC, DOG1 ISH, and PDGFRA ISH—the most distinguishing feature was PDGFRA ISH positivity (FIG. 2), with overexpression of PDGFRA by PDFGRA ISH seen in only in a small subset of GISTs. Images of all cores from both TMAs were digitally captured and are available at the website: microarray-pubs.stanford.edu/tma_portal/dog1.

From the 460 tumor samples that were not classified as GISTs in the first TMA, only four cases that were not histologically and immunophenotypically consistent with GIST were immunoreactive with DOG1 antiserum: one synovial sarcoma ( 1/20=5%), one leiomyosarcoma ( 1/40=2.5%), one fibrosarcoma (¼=25%), and one Ewing's sarcoma/PNET ( 1/9=11%). Of the 40 leiomyosarcomas, 17 originated in the abdomen and none of these were DOG1 immunoreactive. Other tumors in the GIST differential diagnosis failed to stain with the DOG1 antisera. These include desmoid fibromatosis (17 cases) and Schwannoma (3 cases). Under the staining conditions used, none of the fibromatosis cases were positive for KIT by immunohistochemistry or in situ hybridization. One leiomyosarcoma was positive for KIT immunohistochemistry only (TMA 3725). The staining was exclusively in a diffuse nuclear pattern. This tumor was negative for DOG1 by both immunohistochemistry and in situ hybridization and for KIT in situ hybridization.

Seven cases in the first TMA, not counted among the 22 unequivocal GISTs, showed histologic features indeterminate between GIST and smooth muscle tumor. All of these tumors were located in the wall of the stomach or intestine, with four tumors from the stomach, one from the duodenum, one from the gastro-esophageal junction, and one from the rectum. All seven cases were negative for KIT by immunohistochemistry and thus might not be considered GISTs according to current recommendations⁶. However, four of the seven cases were positive by KIT in situ hybridization, while DOG1 immunoreactivity was seen in two cases, and all seven cases were positive for DOG1 by in situ hybridization. Furthermore, two cases (TMA 863 and 3696) were positive for PDGFRA in situ hybridization. Subsequent sequence analysis of cases 863 and 3696 revealed a point mutation and a deletion in exon 18 of PDGFRA, respectively. To date, such mutations have only been described in GISTs. We conclude that the seven KIT immunonegative cases with morphologic features between GIST and smooth muscle tumor actually represent GISTs.

A tissue microarray containing a spectrum of normal tissues was also stained with the DOG1 antiserum. Staining in the epithelium of breast, prostate, salivary gland, liver, stomach, testis, pancreas, and gallbladder was observed. The pattern of DOG1 immunostaining of the interstitial cells of Cajal was similar to KIT. In addition, DOG1 antiserum reacted with a number of tumor cores in a carcinoma array, including some that did not stain with KIT antiserum.

Tables

Table 1 summarizes the staining results that were obtained with different genotypic TMA samples using KIT IHC, KIT ISH, PDGFRA ISH, DOG1 IHC, and DOG1 ISH (see also FIG. 3).

TABLE 1

| | KIT | KIT | PDGFRA | DOG1 | DOG1 | |
|---|---|---|---|---|---|---|
| Genotype | IHC | ISH | ISH | IHC | ISH | |
| ¹Wild- | 14 | 10 | 9 | 14 | 3 | total scorables |
| type | 14 | 9 | 1 | 14 | 3 | total positive |
| | 100 | 90 | 11 | 100 | 100 | % positive |
| ²KIT | 9 | 7 | 7 | 9 | 6 | total scorables |
| ex 9 | 9 | 6 | 2 | 8 | 5 | total positive |
| | 100 | 86 | 29 | 89 | 83 | % positive |
| ²KIT | 86 | 57 | 51 | 81 | 39 | total scorables |
| ex 11 | 82 | 47 | 6 | 81 | 38 | total positive |
| | 95 | 82 | 12 | 100 | 97 | % positive |
| ²KIT | 3 | 3 | 2 | 3 | 2 | total scorables |
| ex 13 | 3 | 2 | 1 | 3 | 2 | total positive |
| | 100 | 67 | 50 | 100 | 100 | % positive |
| ²KIT | 1 | 1 | 1 | 1 | 0 | total scorables |
| ex 17 | 1 | 1 | 0 | 1 | 0 | total positive |
| | 100 | 100 | 0 | 100 | NA | % positive |
| ³PDGFRA | 8 | 7 | 6 | 8 | 7 | total scorables |
| ex 12 | 3 | 1 | 5 | 8 | 5 | total positive |
| or 18 | 37.5 | 14 | 83 | 100 | 71 | % positive |
| Unknown | 23 | 23 | 21 | 23 | 23 | total scorables |
| | 22 | 21 | 8 | 21 | 22 | total positive |
| | 96 | 91 | 38 | 91 | 96 | % positive |

¹These GISTs belong to the KIT negative, PDGFRA negative subclass.
²These GISTs belong to the KIT positive subclass.
³These GISTs belong to the PDGFRA positive subclass.

Discussion

In this Example, it has been demonstrated that the gene, DOG1, identified in a DNA microarray analysis of gene expression patterns as associated with GIST, is highly expressed in KIT mutant positive GISTs, PDGFRA mutant positive GISTs and KIT mutant negative, PDGFRA mutant negative (“wild-type”) GISTs. Expression of DOG1 in GISTs was demonstrated both by immunodetection of the protein and by in situ hybridization. DOG1 immunoreactivity was assessed on two soft tissue tumor microarrays representing 587 soft tissue tumors, including 149 GISTs. 98.7% of scorable GISTs demonstrated immunoreactivity with DOG1 antisera. Only four KIT mutant negative, non-GIST soft tissue tumors were DOG1 immunoreactive. Several GISTs with mutations in the PDGFRA gene were found to react only by in situ hybridization for DOG1 and to be negative for DOG1 by immunohistochemistry. It is anticipated that monoclonal antibodies against purified DOG1 will yield tools with sensitivity similar to that seen with in situ hybridization probes. The inventors have also confirmed PDGFRA expression in a subset of GISTs using in situ hybridization. PDGFRA expression and KIT expression are not mutually exclusive. A subset of KIT mutant positive GISTs expresses PDGFRA in addition to KIT while a subset of PDGFRA mutant positive tumors also expresses KIT. These data were seen with both immunohistochemical and in situ hybridization techniques.

In addition to the marked similarity in reactivity for DOG1 protein on non-GIST sarcomas, DOG1 protein can also be seen in a subset of melanomas and germ cell tumors as has been described for KIT (data not shown). Furthermore just as seen with the KIT molecule, a variety of carcinomas also express DOG1. These tumors mostly overlap with the KIT positive tumors. While within the field of soft tissue tumors DOG1 expression appears quite specific for GIST, in a differential diagnostic setting DOG1 reactivity does not exclude carcinomas. Therefore additional markers such as keratin stains could be advantageously used when the differential diagnosis includes carcinoma.

The inventors have also demonstrated the feasibility of assessing GIST markers by in situ hybridization on paraffin embedded tissue. Correlation between IHC and ISH for DOG1 on GISTs was excellent. In the case of KIT, the correlation was not as strong due to relatively weak or absent ISH signals in some KIT mutant positive GISTs. It is likely that this reflects lower sensitivity of the KIT ISH assay, although cross-reactivity of the KIT antibody to another epitope on GISTs has not been excluded. In situ hybridization for PDGFRA proved to be valuable in identifying KIT mutant negative GISTs, although DOG1 immunohistochemistry was equally sensitive for these cases. Overall, the inventors have found that ISH techniques are complementary to IHC tests in the evaluation of GISTs.

As noted previously, DOG1 has been recently identified as a gene in the CCND1-EMS1 locus on human chromosome 11q13 and officially named transmembrane protein 16A (TMEM16A, see GenBank GeneID for DOG1 is 5510)²⁶. The CCND1-EMS1 locus has been shown to be amplified in a number of human cancers including squamous cell carcinomas of the head and neck³⁵; hepatocellular carcinoma³⁶; esophageal squamous cell carcinomas³⁷; breast cancer³⁸⁻⁴⁰; and ovarian carcinoma⁴¹. Human DOG1 protein showed 89.8% total-amino-acid identity with mouse DOG1 protein, and also 58.4%, 38.3%, and 38.6% identity with human C12orf3, C11orf25, and FLJ34272/BAC03704 proteins, respectively²⁶. Sequence analysis predicts the presence of eight transmembrane spanning segments. This correlates with observations of the immunohistochemical localization to the cell membrane. DOG1 may be part of an as yet unclassified ion transporter family²⁶.

### REFERENCES

1. Weiss S W, Goldblum J R: Soft tissue tumors. St Louis, Mosby, 2001
2. Heinrich M C, Corless C, Demetri G D, Blanke C, von Mehren M, Joensuu H, McGreevey L, Chen C J, Van den Abbeele A, Druker B, Kiese B, Eisenberg B, Roberts P, Singer S, Fletcher C D, Silberman S, Dimitrijevic S, Fletcher J A: Kinase Mutations and Imatinib response in patients with metastatic gastrointestinal stromal tumor. J Clin Oncol 2003, 21:4342-4349
3. van Oosterom A T, Judson I, Verweij J, Stroobants S, Donato di Paola E, Dimitrijevic S, Martens M, Webb A, Sciot R, Van Glabbeke M, Silberman S, Nielsen O S, EORTC Soft Tissue and Bone Sarcoma Group: Safety and efficacy of imatinib (ST1571) in metastatic gastrointestinal stromal tumours: a phase I study. Lancet 2001, 358:1421-1423
4. Demetri G, von Mehren M, Blanke C, Van den Abbeele A, Eisenberg B, Roberts P, Heinrich M, Tuveson D, Singer S, Janicek M, Fletcher J, Silverman S, Silberman S, Capdeville R, Kiese B, Peng B, Dimitrijevic S, Druker B, Corless C, Fletcher C, Joensuu H: Efficacy and safety of imatinib mesylate in advanced gastrointestinal stromal tumors. N Engl J Med 2002, 347:472-480
5. Joensuu H, Roberts P, Sarlomo-Rikala M, Andersson L, Tervahartiala P, Tuveson D, Silberman S, Capdeville R, Dimitrijevic S, Druker B, Demetri G: Effect of the tyrosine kinase inhibitor STI571 in a patient with a metastatic gastrointestinal stromal tumor. N Engl J Med 2001, 344:1052-1056
6. Fletcher C D, Berman J J, Corless C, Gorstein F, Lasota J, Longley B J, Miettinen M, O'Leary T J, Remotti H, Rubin B P, Shmookler B, Sobin L H, Weiss S W: Diagnosis of gastrointestinal stromal tumors: A consensus approach. Hum Pathol 2002, 33:459-465
7. van de Rijn M, Rouse R V: CD34: a review. Applied Immunohistochemistry 1994, 2:71-
8. van de Rijn M, Hendrickson M R, Rouse R V: CD34 expression by gastrointestinal tract stromal tumors. Hum Pathol 1994, 25:766-771
9. Yantiss R K, Spiro I J, Compton C C, Rosenberg A E: Gastrointestinal stromal tumor versus intra-abdominal fibromatosis of the bowel wall: a clinically important differential diagnosis. Am J Surg Pathol 2000, 24:947-957
10. Smithey B E, Pappo A S, Hill D A: C-kit expression in pediatric solid tumors: a comparative immunohistochemical study. Am J Surg Pathol 2002, 26:486-492
11. Hirota S, Nishida T, Isozaki K, Taniguchi M, Nakamura J, Okazaki T, Kitamura Y: Gain-of-function mutation at the extracellular domain of KIT in gastrointestinal stromal tumours. J Pathol 2001, 193:505-510
12. Rubin B, Singer S, Tsao C, Duensing A, Lux M, Ruiz R, Hibbard M, Chen C, Xiao S, Tuveson D, Demetri G, Fletcher C, Fletcher J: KIT activation is a ubiquitous feature of gastrointestinal stromal tumors. Cancer Res 2001, 61:8118-8121
13. Heinrich M C, Corless C L, Duensing A, McGreevey L, Chen C J, Joseph N, Singer S, Griffith D J, Haley A, Town A, Demetri G D, Fletcher C D, Fletcher J A: PDGFRA activating mutations in gastrointestinal stromal tumors. Science 2003, 299:708-710
14. Hirota S, Ohashi A, Nishida T, Isozaki K, Kinoshita K, Shinomura Y, Kitamura Y: Gain-of-function mutations of platelet-derived growth factor receptor alpha gene in gastrointestinal stromal tumors. Gastroenterology 2003, 125:660-667
15. Bauer S, Corless C, Heinrich M, Dirsch O, Antoch G, Kanja J, Seeber S, Schutte J: Response to imatinib mesylate of a gastrointestinal stromal tumor with very low expression of KIT. Cancer Chemother Pharmacol 2003, 51:261-265
16. Allander S V, Nupponen N N, Ringner M, Hostetter G, Maher G W, Goldberger N, Chen Y, Carpten J, Elkahloun A G, Meltzer P S: Gastrointestinal stromal tumors with KIT mutations exhibit a remarkably homogeneous gene expression profile. Cancer Res 2001, 61:8624-8628
17. Khan J, Wei J S, Ringner M, Saal L H, Ladanyi M, Westermann F, Berthold F, Schwab M, Antonescu C R, Peterson C, Meltzer P S: Classification and diagnostic prediction of cancers using gene expression profiling and artificial neural networks. Nat Med 2001, 7:673-679
18. Nielsen T O, West R B, Linn S C, Alter O, Knowling M A, O'Connell J X, Zhu S, Fero M, Sherlock G, Pollack J R, Brown P O, Botstein D, van de Rijn M: Molecular characterisation of soft tissue tumours: a gene expression study. Lancet 2002, 359:1301-
19. Kononen J, Bubendorf L, Kallioniemi A, Barlund M, Schraml P, Leighton S, Torhorst J, Mihatsch M J, Sauter G, Kallioniemi O P: Tissue microarrays for high-throughput molecular profiling of tumor specimens. 1998, 4:844-847
20. West R B, Harvell J, Linn S, Lui C, Prapong W, Hernandez-Boussard T, Montgomery K, Nielsen T O, Rubin B P, Patel R, Goldblum J R, Brown P, van de Rijn M: Apo D in Soft Tissue tumors: a novel marker for dermatofibrosarcoma protuberans. Am J Surg Pathol In press
21. Torhorst J, Bucher C, Kononen J, Haas P, Zuber M, Kochli O, Mross F, Dieterich H, Moch H, Mihatsch M, Kallioniemi O, Sauter G: Tissue microarrays for rapid linking of molecular changes to clinical endpoints. Am J Pathol 2001, 159:2249-2256
22. Corless C, McGreevey L, Haley A, Town A, Heinrich M: KIT mutations are common in incidental gastrointestinal stromal tumors one centimeter or less in size. Am J Pathol 2002, 160:1567-1572
23. St Croix B, Rago C, Velculescu V, Traverso G, Romans K, Montgomery E, Lal A, Riggins G, Lengauer C, Vogelstein B, Kinzler K: Genes expressed in human tumor endothelium. Science 2000, 289:1197-1202
24. Iacobuzio-Donahue C A, Ryu B, Hruban R H, Kern S E: Exploring the host desmoplastic response to pancreatic carcinoma: gene expression of stromal and neoplastic cells at the site of primary invasion. Am J Pathol 2002, 160:91-99
25. Liu C L, Prapong W, Natkunam Y, Alizadeh A, Montgomery K, Gilks C B, van de Rijn M: Software tools for high-throughput analysis and archiving of immunohistochemistry staining data obtained with tissue microarrays. Am J Pathol 2002, 161:1557-1565
26. Katoh M, Katoh M: FLJ10261 gene, located within the CCND1-EMS1 locus on human chromosome 11q13, encodes the eight-transmembrane protein homologous to C12orf3, C11orf25 and FLJ34272 gene products. Int J Oncol 2003, 22:1375-1381
27. Medeiros F, Corless C L, Duensing A, Hornick J L, Oliveira A M, Heinrich M C, Fletcher J A, Fletcher C D: KIT-negative gastrointestinal stromal tumors: proof of concept and therapeutic implications. Am J Surg Pathol. 2004, 28(7):889-894
28. Ohashi A, Kinoshita K, Isozaki K, Nishida T, Shinomura Y, Kitamura Y, Hirota S: Different inhibitory effect of imatinib on phosphorylation of mitogen-activated protein kinase and Akt and on proliferation in cells expressing different types of mutant platelet-derived growth factor receptor-alpha. Int J Cancer. 2004, 111(3):317-21
29. Joensuu H, Kindblom L G: Gastrointestinal stromal tumors—a review. Acta Orthop Scand Suppl. 2004, 75(311):62-71
30. Wasag B, Debiec-Rychter M, Pauwels P, Stul M, Vranckx H, Oosterom A V, Hagemeijer A, Sciot R: Differential expression of KIT/PDGFRA mutant isoforms in epithelioid and mixed variants of gastrointestinal stromal tumors depends predominantly on the tumor site. Mod Pathol. 2004 May 21 [Epub ahead of print]
31. Lasota J, Dansonka-Mieszkowska A, Sobin L H, Miettinen M: A great majority of GISTs with PDGFRA mutations represent gastric tumors of low or no malignant potential. Lab Invest. 2004, 84(7):874-83
32. Debiec-Rychter M, Wasag B, Stul M, De Wever I, Van Oosterom A, Hagemeijer A, Sciot R: Gastrointestinal stromal tumours (GISTs) negative for KIT (CD117 antigen) immunoreactivity. J Pathol. 2004, 202(4):430-8
33. Yamamoto H, Oda Y, Kawaguchi K, Nakamura N, Takahira T, Tamiya S, Saito T, Oshiro Y, Ohta M, Yao T, Tsuneyoshi M: c-kit and PDGFRA mutations in extragastrointestinal stromal tumor (gastrointestinal stromal tumor of the soft tissue). Am J Surg Pathol. 2004, 28(4):479-88
34. Debiec-Rychter M, Dumez H, Judson I, Wasag B, Verweij J, Brown M, Dimitrijevic S, Sciot R, Stul M, Vranck H, Scurr M, Hagemeijer A, van Glabbeke M, van Oosterom AT; EORTC Soft Tissue and Bone Sarcoma Group: Use of c-KIT/PDGFRA mutational analysis to predict the clinical response to imatinib in patients with advanced gastrointestinal stromal tumours entered on phase I and II studies of the EORTC Soft Tissue and Bone Sarcoma Group. Eur J Cancer. 2004, 40(5):689-95
35. Rodrigo J P, Garcia L A, Ramos S, Lazo P S, Suarez C: EMS1 gene amplification correlates with poor prognosis in squamous cell carcinomas of the head and neck. Clin Cancer Research. 2000, 6:3177-3182-95
36. Yuan B Z, Zhou X, Zimonjic D B, Durkin M E, Popescu N C: Amplification and overexpression of the EMS1 oncogene, a possible prognostic marker, in human hepatocellular carcinoma. J Molec Diagnostics. 2003, 5(1):48-53
37. Janssen J W G, Imoto I, Inoue J, Shimada Y, Ueda M, Imamura M, Bartram C R, Inazawa J: MYEOV, a gene at 11q13, is coamplified with CCDN1, but epigenetically inactivated in a subset of esophageal squamous cell carcinomas. J Hum Genet. 2002, 47:460-464
38. Jui R, Campbell D H, Lee C S L, McCaul K, Horsfall D J, Musgrove E A, Daly R J, Seshadri R, Sutherland R L: EMS1 amplification can occur independently of CCDN1 or INT-2 amplification at 11q13 and may identify different phenotypes in primary breast cancer. Oncogene. 1997, 15:1617-1623
39. Janssen J W G, Cuny M, Orsetti B, Rodriguez C, Valles H, Bartram C R, Schuuring E, Theillet C: MYEOV: a candidate gene for DNA amplification events occurring centromeric to CCND1 in breast cancer. Int J Cancer. 2002, 102:608-614
40. Schrmal P, Schwerdtfeger G, Burhalter F, Raggi A, Schmidt D, Ruffalo T, King W, Wilber K, Mihatsch M J, Moch H: Combined array comparative genomic hybridization and tissue microarray analysis suggest PAK1 at 11q13.5-q14 as a critical oncogene target in ovarian carcinoma. Am J Pathol. 2003, 163:985-992
41. Ormandy C J, Musgrove E A, Hui R, Daly R J, Sutherland R L: Cyclin D1, EMS 1 and 11q13 amplification in breast cancer. Breast Cancer Res and Treat. 2003, 78:323-335
42. Fabbro D, Ruetz S, Bodis S, Pruschy M, Csermak K, Man A, Campochiaro P, Wood J, O'Reilly T, Meyer T: PKC412—a protein kinase inhibitor with a broad therapeutic potential. Anticancer Drug Des. 2000, 15(1):17-28

### Other Embodiments

Other embodiments of the invention will be apparent to those skilled in the art from a consideration of the specification or practice of the invention disclosed herein. It is intended that the specification and Examples be considered as exemplary only, with the true scope of the invention being indicated by the following claims.

We claim:

1. A method comprising steps of providing a tumor sample; detecting expression or activity of a gene encoding a DOG1polypeptide in the sample; and classifying the tumor as a gastrointestinal stromal tumor belonging to a PDGFRA positive subclass based on the results of the detecting step.

2. The method of claim 1 further comprising a step of detecting expression or activity of a gene encoding a KIT polypeptide in the sample, wherein the classifying step is based on the results of both detecting steps.

3. The method of claim 1 further comprising a step of detecting expression or activity of a gene encoding a PDGFRA polypeptide in the sample, wherein the classifying step is based on the results of both detecting steps.

4. The method of claim 1 further comprising steps of detecting expression or activity of a gene encoding a KIT polypeptide in the sample; and detecting expression or activity of a gene encoding a PDGFRA polypeptide in the sample, wherein the classifying step is based on the results of all three detecting steps.

5. A method comprising steps of providing a tumor sample; detecting expression or activity of a gene encoding a DOG1 polypeptide in the sample; and classifying the tumor as a gastrointestinal stromal tumor belonging to a KIT negative, PDGFRA negative subclass based on the results of the detecting step.

6. The method of claim 5 further comprising a step of detecting expression or activity of a gene encoding a KIT polypeptide in the sample, wherein the classifying step is based on the results of both detecting steps.

7. The method of claim 5 further comprising a step of detecting expression or activity of a gene encoding a PDGFRA polypeptide in the sample, wherein the classifying step is based on the results of both detecting steps.

8. The method of claim 5 further comprising steps of detecting expression or activity of a gene encoding a KIT polypeptide in the sample; and detecting expression or activity of a gene encoding a PDGFRA polypeptide in the sample, wherein the classifying step is based on the results of all three detecting steps.

9. The method of claims 1 or 5, wherein the tumor sample is isolated from a subject having a tumor, the method further comprising a step of providing diagnostic, prognostic, or predictive information about the subject based on the results of the classifying step.

10. The method of claims 1 or 5, wherein the tumor sample is isolated from a subject having a tumor, the method further comprising a step of stratifying the subject for a clinical trial based on the results of the classifying step.

11. The method of claims 1 or 5, wherein the tumor sample is isolated from a subject having a tumor, the method further comprising a step of selecting a treatment based on the results of the classifying step.

12. The method of claims 1 or 5, wherein the step of detecting expression or activity of the gene encoding a DOG1 polypeptide comprises detecting a DOG1 polypeptide.

13. The method of claim 2, 4, 6 or 8, wherein the step of detecting expression or activity of the gene encoding a KIT polypeptide comprises detecting a KIT polypeptide.

14. The method of claim 3, 4, 7 or 8, wherein the step of detecting expression or activity of the gene encoding a PDGFRA polypeptide comprises detecting a PDGFRA polypeptide.

15. The method of claim 12, wherein the polypeptide or fragment is detected by performing immunohistochemical analysis on the sample using an antibody that specifically binds to the polypeptide.

16. The method of claim 13, wherein the polypeptide or fragment is detected by performing immunohistochemical analysis on the sample using an antibody that specifically binds to the polypeptide.

17. The method of claim 14, wherein the polypeptide or fragment is detected by performing immunohistochemical analysis on the sample using an antibody that specifically binds to the polypeptide.

18. The method of claims 1 or 5, wherein the step of detecting expression or activity of the gene encoding a DOG1 polypeptide comprises detecting a nucleotide molecule encoding a DOG1 polypeptide.

19. The method of claim 2, 4, 6 or 8, wherein the step of detecting expression or activity of the gene encoding a KIT polypeptide comprises detecting a nucleotide molecule encoding a KIT polypeptide.

20. The method of claim 3, 4, 7 or 8, wherein the step of detecting expression or activity of the gene encoding a PDGFRA polypeptide comprises detecting a nucleotide molecule encoding a PDGFRA polypeptide.

21. The method of claim 18, wherein the nucleotide molecule is detected by in situ hybridization.

22. The method of claim 19, wherein the nucleotide molecule is detected by in situ hybridization.

23. The method of claim 20, wherein the nucleotide molecule is detected by in situ hybridization.

24. The method of claims 1 or 5, wherein the tumor sample is selected from the group consisting of a blood sample, a urine sample, a serum sample, an ascites sample, a saliva sample, a cell, and a portion of tumor tissue.

25. The method of claim 24, wherein the tumor sample is a portion of tumor tissue.

26. The method of claims 1 or 5 further comprising a step of determining the specific mutations within the KIT and/or PDFGRA genes in the sample.

27. The method of claim 11, wherein the selected treatment is imatinib therapy.

28. A kit for use in classifying tumors, the kit comprising one or more antibodies for a DOG1 polypeptide; and instructions for use of the kit.

29. The kit of claim 28 further comprising a control slide comprising tumor samples for testing reagents in the kit.

30. The kit of claim 29 further comprising one or more antibodies for a KIT polypeptide and/or a PDGFRA polypeptide, wherein the control slide comprises gastrointestinal stromal tumor samples.

31. The kit of claim 29 or 30 for use in classifying gastrointestinal stromal tumors as belonging to a PDGFRA positive subclass, wherein the control slide comprises gastrointestinal stromal tumor samples.

32. The kit of claim 29 or 30 for use in classifying gastrointestinal stromal tumors as belonging to a KIT negative, PDGFRA negative subclass, wherein the control slide comprises gastrointestinal stromal tumor samples.

33. A kit for use in classifying tumors, the kit comprising one or more primers or probes for use in detecting expression of a gene encoding a DOG1 polypeptide; and instructions for use of the kit.

34. The kit of claim 33 further comprising a control slide comprising tumor samples for testing reagents in the kit.

35. The kit of claim 34 further comprising one or more primers or probes for use in detecting expression of a gene encoding a KIT polypeptide and/or a gene encoding a PDGFRA polypeptide, wherein the control slide comprises gastrointestinal stromal tumor samples.

36. The kit of claim 33 or 34 for use in classifying gastrointestinal stromal tumors as belonging to a PDGFRA positive subclass, wherein the control slide comprises gastrointestinal stromal tumor samples.

37. The kit of claim 34 or 35 for use in classifying gastrointestinal stromal tumors as belonging to a KIT negative, PDGFRA negative subclass, wherein the control slide comprises gastrointestinal stromal tumor samples.
